(19) United States
(12) Patent Application Publication
(10) Pub. No.: US 2026/0022123 A1
(43) Pub. Date: Jan. 22, 2026

(54) Methods of Making Modified BTK Inhibitors

(71) Applicant: Principia Biopharma Inc., Bridgewater, NJ (US)

(72) Inventors: John L. Kane, JR., Cambridge, MA (US); Timothy D. Owens, Cambridge, MA (US); Timothy J. Turner, Belmont, MA (US)

(73) Assignee: Principia Biopharma Inc., Bridgewater, NJ (US)

(21) Appl. No.: 18/875,830

(22) PCT Filed: Jun. 21, 2023
(86) PCT No.: PCT/US2023/025808

§ 371 Date: Dec. 17, 2024

Related U.S. Application Data
(60) Provisional application No. 63/354,330, filed on Jun. 22, 2022.

(51) Int. Cl. C07D 487/04; A61K 31/454; A61K 31/4545; A61K 31/496; A61K 31/506; A61K 31/519; C07D 401/04; C07D 401/10; C07D 401/12; C07D 471/04

(52) U.S. Cl. CPC C07D 487/04; A61K 31/454; A61K 31/4545; A61K 31/496; A61K 31/506; A61K 31/519; C07D 401/04; C07D 401/10; C07D 401/12; C07D 471/04

(57) ABSTRACT

Provided herein are BTK inhibitors containing piperidine modified at the 3-position. Further disclosed are methods of making and using said BTK inhibitors.

### FIELD

The present disclosure relates to BTK inhibitors and methods for preparing and using same, wherein the BTK inhibitor has a piperidine ring substituted at the 3-position, for example, the BTK inhibitor can be chosen from Compounds of Formula (1)-(9):

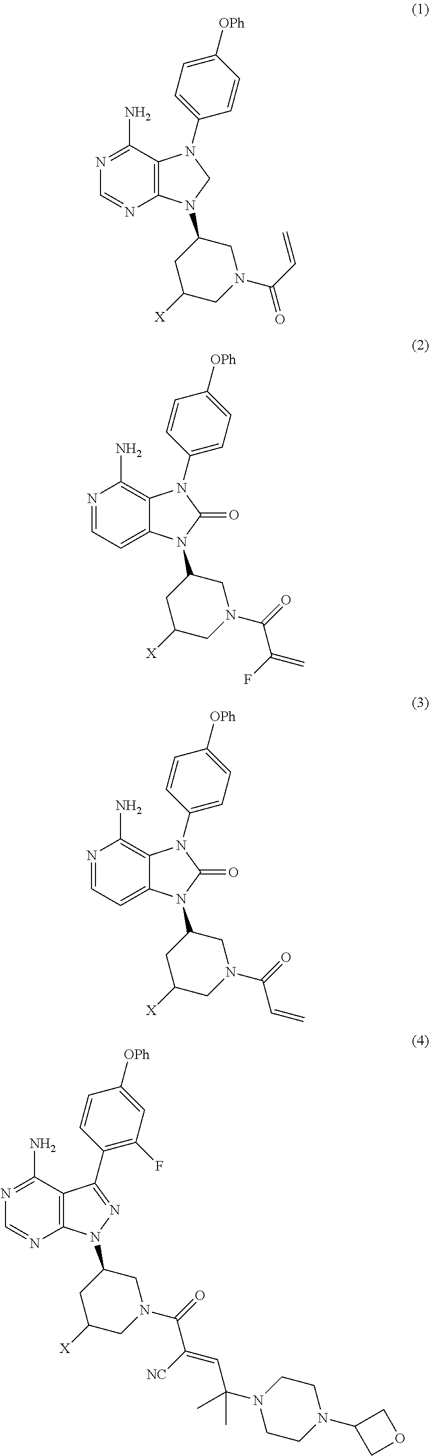

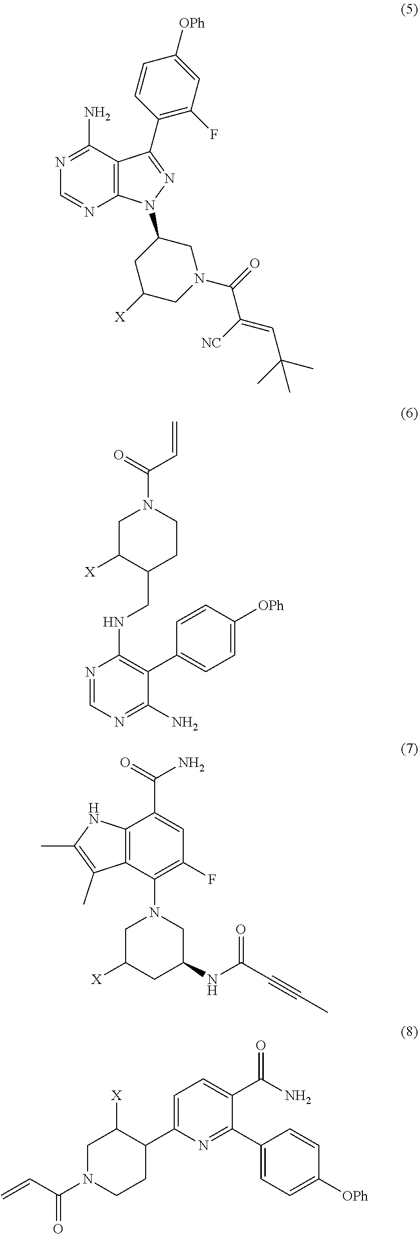

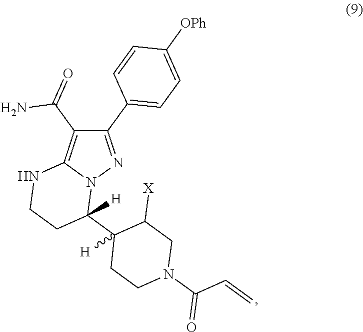

wherein X is chosen from —OH, —OY, —Y₂, and -(halo)₂, and Y is C₁-C₄ alkyl optionally substituted with 1 to 3 halo. The Compounds of Formula (1)-(9) are Bruton's Tyrosine Kinase (BTK) inhibitors and thus can be useful in the treatment of diseases or disorders resulting from an excess of BTK signaling, for example, a disease selected from an autoimmune disease, an inflammatory disease, or cancer.

### BACKGROUND AND SUMMARY

The enzyme BTK is a member of the Tec family non-receptor tyrosine kinases. BTK is expressed in most hematopoietic cells including B cells, mast cells, and macrophages. BTK plays a role in the development and activation of B cells. BTK activity has been implicated in the pathogenesis of several disorders and conditions including B cell-related hematological cancers (such as non-Hodgkin lymphoma and B cell chronic lymphocytic leukemia) and autoimmune diseases (such as multiple sclerosis, rheumatoid arthritis, lupus, immune thrombocytopenia (ITP), rheumatoid arthritis, Sjogren's syndrome, pemphigus, inflammatory bowel disease (IBD), lupus nephritis, atopic dermatitis, warm autoimmune hemolytic anemia, asthma and other acute respiratory distress, and chronic spontaneous urticaria).

Accordingly, pharmaceutical compositions comprising therapeutically-effective amounts of BTK inhibitors may be useful in the treatment of certain cancers and autoimmune diseases.

When treating certain cancers and autoimmune diseases with BTK inhibitors, it is also desirable that the treatment can be administered in a form that is easily absorbed by the body and also shelf stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency of the composition.

One factor in the suitability of an inhibitor compound as a therapeutic agent is its ability to interact with the targeted binding site of the protein allowing for at least one of more selective, specific, and stronger binding. The metabolization of piperidine-containing BTK inhibitors can cause the piperidine to undergo ring opening, which may reduce its binding affinity to the C481 sulfhydryl, resulting in overall decreased drug efficacy. Therefore, structural modifications of BTK inhibitors that can prevent or modify this ring opening process is of interest, for example, certain substitutions at the 3-position of the piperidine moiety. Although not bound by theory, it is believed that by providing a substitution at the 3-position of piperidine, this substituent provides stability and/or rigidity to the ring structure that may prevent metabolization and/or degradation of the ring structure.

Thus, the present disclosure relates to BTK inhibitors and methods for preparing and using same, wherein the BTK inhibitor has a piperidine ring substituted at the 3-position, for example, the BTK inhibitor can be chosen from Compounds of Formula (1)-(9):

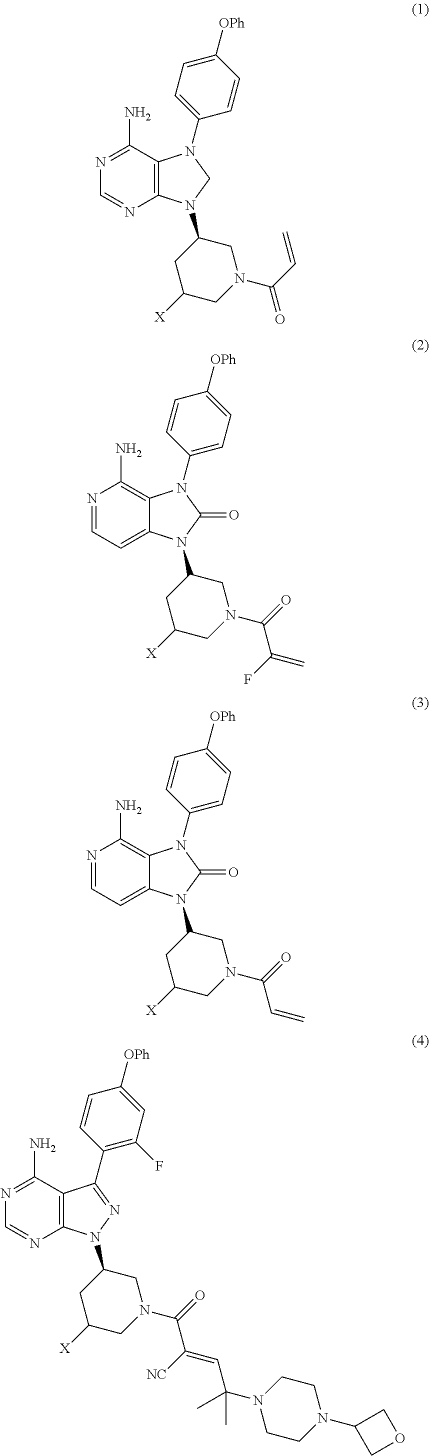

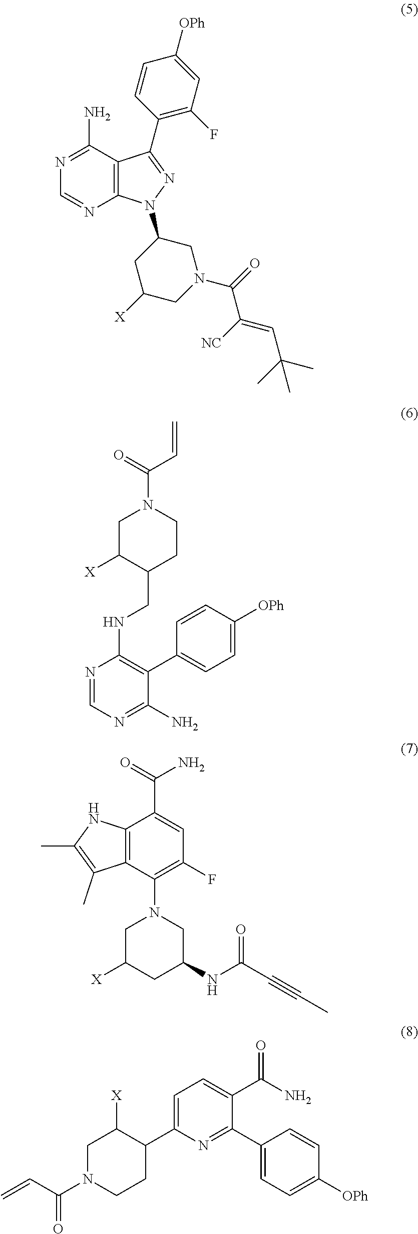

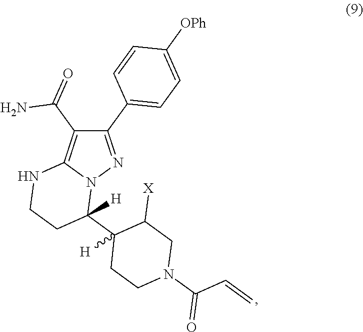

wherein X is chosen from —OH, —OY, —Y₂, and -(halo)₂, and Y is C₁-C₄ alkyl optionally substituted with 1 to 3 halo, and methods of preparing said compound. The present disclosure also relates to a composition comprising a pharmaceutically acceptable excipient and at least one compound chosen from those disclosed herein. The present disclosure still further relates to method of treating a disease or disorder mediated by BTK comprising administering to a patient in need thereof an effective amount of a compound chosen from those disclosed herein.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### DETAILED DESCRIPTION

### Definitions

Unless otherwise stated, the following terms used in the specification and claims are defined for the purposes of this disclosure and have the following meaning:

As used herein, “the BTK inhibitor,” “the BTK inhibitor compound,” “the compound of Formula (1)-(9),” and “Compound (1)-(9),” refers to BTK inhibitors having the following structure:

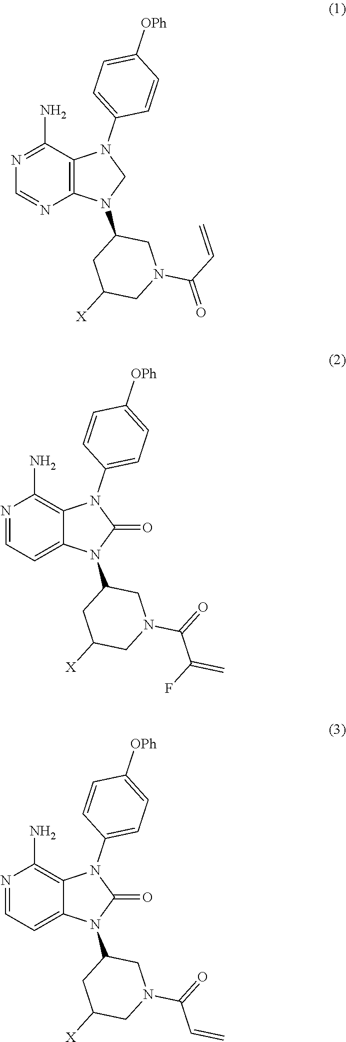

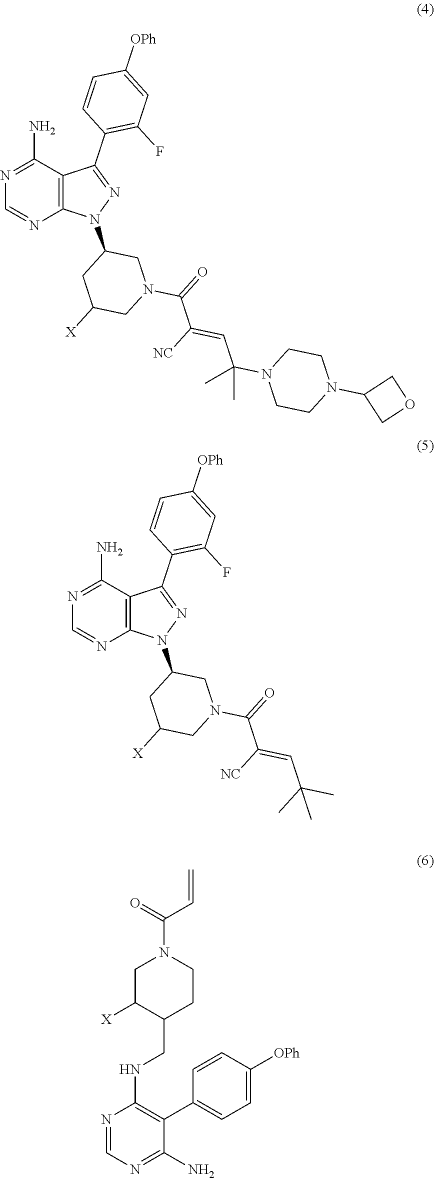

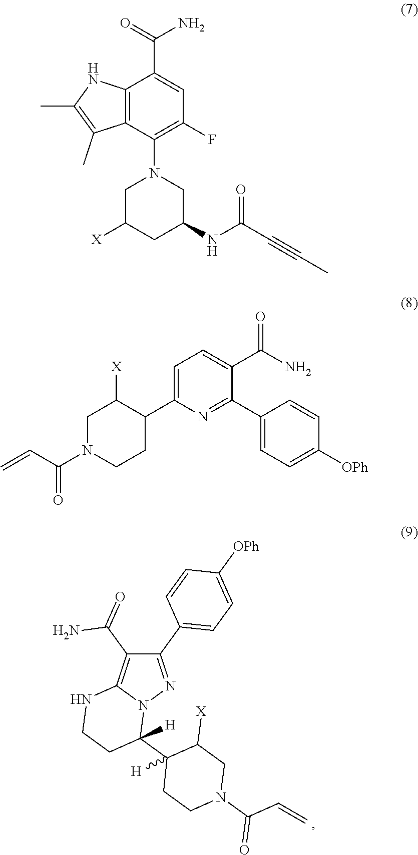

wherein X is chosen from —OH, —OY, —Y₂, and -(halo)₂, and Y is C₁-C₄ alkyl optionally substituted with 1 to 3 halo.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following. “A; B; C; A and B; A and C; B and C; A, B and C”. An exception to this definition will occur only when a combination of elements, functions, steps or acts are in some way inherently mutually exclusive.

As used herein, “3-position of piperidine” and “piperidine ring substituted at the 3-position means the position notated by an “3” as shown:

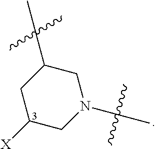

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. A small alkyl group refers to an alkyl group having from 1 to 4 carbons. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “halogen” or “halo” is fluorine, chlorine, bromine or iodine.

An “hydroxy” group is —OH.

An “alkoxy” group is —O-(alkyl), wherein alkyl is defined above.

Embodiments of the disclosure are meant to encompass stereoisomers of the compounds provided herein, such as the compounds of Formula (9).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

It should be noted that if there is a discrepancy between a depicted structure and a name for that structure, the depicted structure is to be accorded more weight.

### Compounds

In some embodiments, the present disclosure relates to compounds of Formula (1):

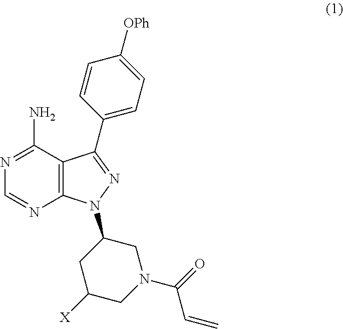

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (2):

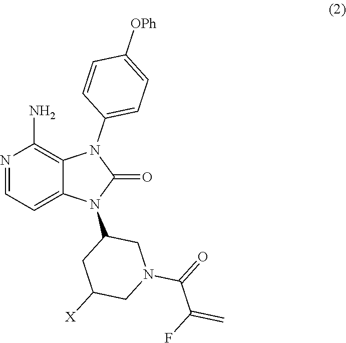

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (3):

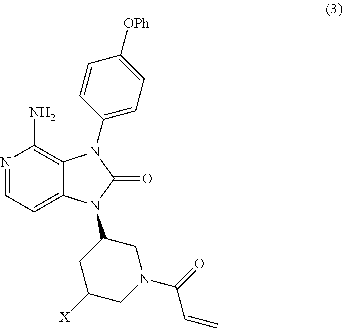

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (4):

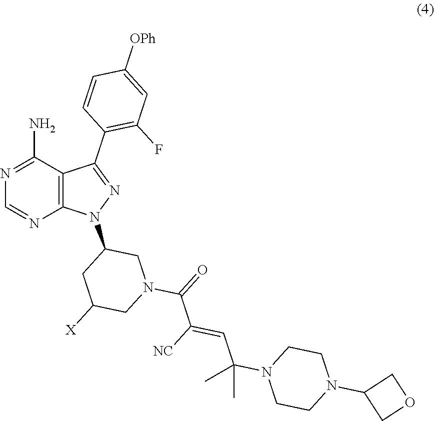

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (5):

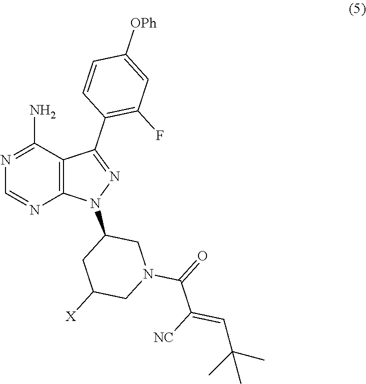

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (6):

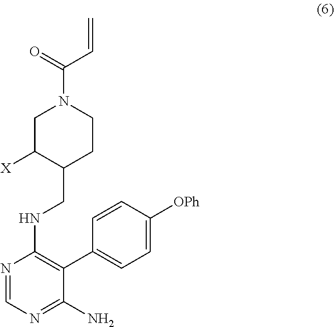

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (7):

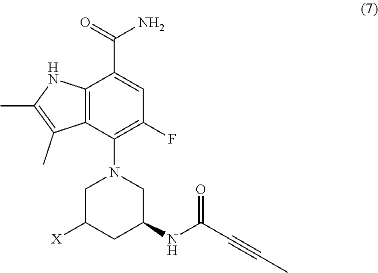

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (8):

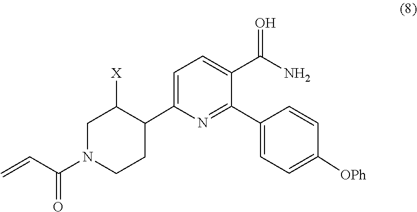

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments, the present disclosure relates to compounds of Formula (9):

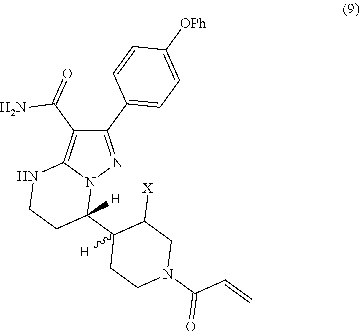

or a pharmaceutically acceptable salt thereof, wherein: X is chosen from —OH, —OY, —Y₂, and -(halo)₂, and Y is C₁-C₄ alkyl optionally substituted with 1 to 3 halo. In some embodiments, Y is chosen from methyl, ethyl, CH(halo)₂, and C(halo)₃. In some embodiments, the halo is F.

In some embodiments the disclosure relates to compounds selected from Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described

TABLE 1

| Compound | | |
|---|---|---|
| No. | Structure | Name |
| 1-A | 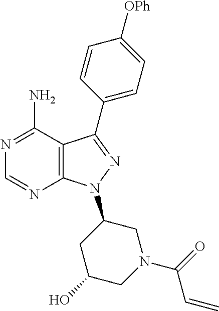 [SMILES: C=CC(=O)N1C[C@H](O)C[C@@H](n2nc(-c3ccc(Oc4ccccc4)cc3)c3c(N)ncnc32)C1] | 1-((3R,5R)-3-(4-amino-3-(4- phenoxyphenyl)-1H- pyrazolo[3,4-d]pyrimidin-1- yl)-5-hydroxypiperidin-1- yl)prop-2-en-1-one |
| 1-B | 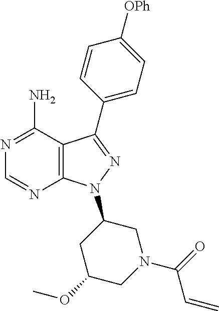 [SMILES: C=CC(=O)N1C[C@H](OC)C[C@@H](n2nc(-c3ccc(Oc4ccccc4)cc3)c3c(N)ncnc32)C1] | 1-((3R,5R)-3-(4-amino-3-(4- phenoxyphenyl)-1H- pyrazolo[3,4-d]pyrimidin-1- yl)-5-methoxypiperidin-1- yl)prop-2-en-1-one |
| 1-C | 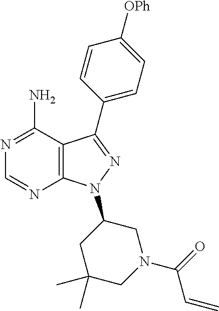 [SMILES: C=CC(=O)N1C[C@H](n2nc(-c3ccc(Oc4ccccc4)cc3)c3c(N)ncnc32)CC(C)(C)C1] | (R)-1-(5-(4-amino-3-(4- phenoxyphenyl)-1H- pyrazolo[3,4-d]pyrimidin-1- yl)-3,3-dimethylpiperidin-1- yl)prop-2-en-1-one |
| 1-D | 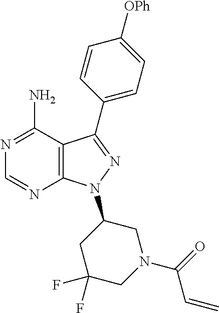 [SMILES: C=CC(=O)N1C[C@H](n2nc(-c3ccc(Oc4ccccc4)cc3)c3c(N)ncnc32)CC(F)(F)C1] | (R)-1-(5-(4-amino-3-(4- phenoxyphenyl)-1H- pyrazolo[3,4-d]pyrimidin-1- yl)-3,3-difluoropiperidin-1- yl)prop-2-en-1-one |
| 2-A | 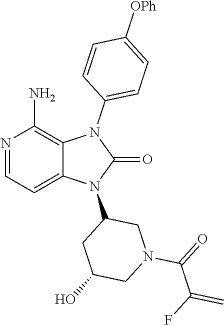 | 4-amino-1-((3S,5R)-1-(2- fluoroacryloyl)-5- hydroxypiperidin-3-yl)-3-(4- phenoxyphenyl)-1,3- dihydro-2H-imidazo[4,5- c]pyridin-2-one |
| 2-B | 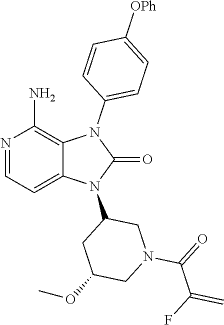 | 4-amino-1-((3S,5R)-1-(2- fluoroacryloyl)-5- methoxypiperidin-3-yl)-3-(4- phenoxyphenyl)-1,3- dihydro-2H-imidazo[4,5- c]pyridin-2-one |
| 2-C | 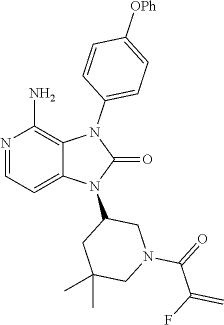 | (S)-4-amino-1-(1-(2- fluoroacryloyl)-5,5- dimethylpiperidin-3-yl)-3- (4-phenoxyphenyl)-1,3- dihydro-2H-imidazo[4,5- c]pyridin-2-one |
| 2-D | 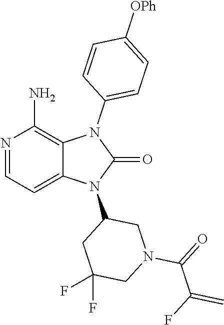 | (S)-4-amino-1-(5,5-difluoro- 1-(2- fluoroacryloyl)piperidin-3- yl)-3-(4-phenoxyphenyl)- 1,3-dihydro-2H-imidazo[4,5- c]pyridin-2-one |
| 3-A | 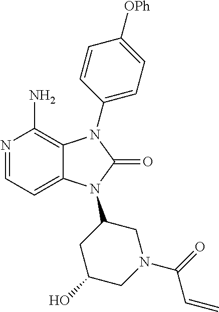 | 1-((3S,5R)-1-acryloyl-5- hydroxypiperidin-3-yl)-4- amino-3-(4-phenoxyphenyl)- 1,3-dihydro-2H-imidazo[4,5- c]pyridin-2-one |
| 3-B | 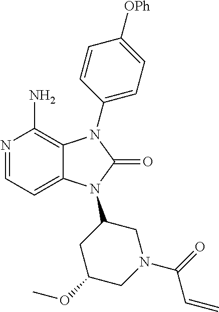 | 1-((3S,5R)-1-acryloyl-5- methoxypiperidin-3-yl)-4- amino-3-(4-phenoxyphenyl)- 1,3-dihydro-2H-imidazo[4,5- c]pyridin-2-one |
| 3-C | 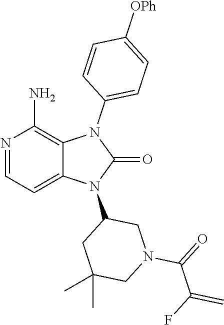 | (S)-4-amino-1-(1-(2- fluoroacryloyl)-5,5- dimethylpiperidin-3-yl)-3- (4-phenoxyphenyl)-1,3- dihydro-2H-imidazo[4,5- c]pyridin-2-one |
| 3-D | 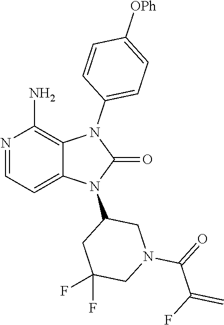 | (S)-4-amino-1-(5,5-difluoro- 1-(2- fluoroacryloyl)piperidin-3- yl)-3-(4-phenoxyphenyl)- 1,3-dihydro-2H-imidazo[4,5- c]pyridin-2-one |
| 4-A | 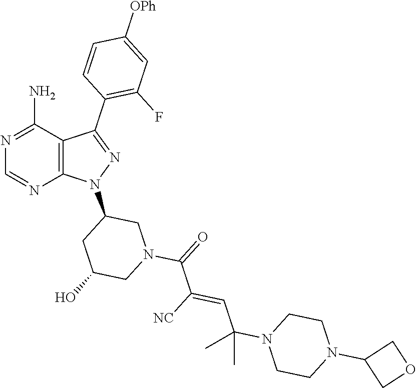 [SMILES: CC(C)(/C=C(\C#N)C(=O)N1C[C@H](O)C[C@@H](n2nc(-c3ccc(Oc4ccccc4)cc3F)c3c(N)ncnc32)C1)N1CCN(C2COC2)CC1] | (E)-2-((3R,5R)-3-(4-amino- 3-(2-fluoro-4- phenoxyphenyl)-1H- pyrazolo[3,4-d]pyrimidin-1- yl)-5-hydroxypiperidine-1- carbonyl)-4-methyl-4-(4- (oxetan-3-yl)piperazin-1- yl)pent-2-enenitrile |
| 4-B | 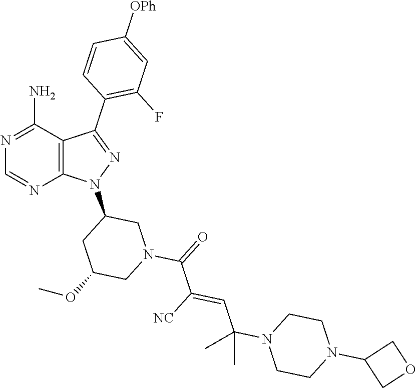 [SMILES: CO[C@@H]1C[C@@H](n2nc(-c3ccc(Oc4ccccc4)cc3F)c3c(N)ncnc32)CN(C(=O)/C(C#N)=C/C(C)(C)N2CCN(C3COC3)CC2)C1] | (E)-2-((3R,5R)-3-(4-amino- 3-(2-fluoro-4- phenoxyphenyl)-1H- pyrazolo[3,4-d]pyrimidin-1- yl)-5-methoxypiperidine-1- carbonyl)-4-methyl-4-(4- (oxetan-3-yl)piperazin-1- yl)pent-2-enenitrile |
| 4-C | 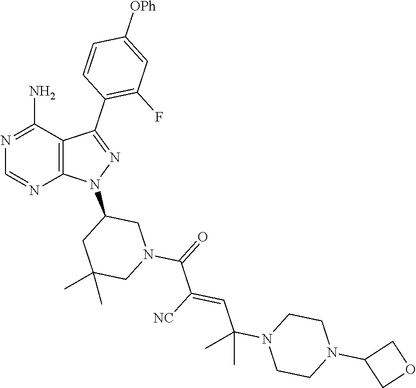 [SMILES: CC1(C)C[C@@H](n2nc(-c3ccc(Oc4ccccc4)cc3F)c3c(N)ncnc32)CN(C(=O)/C(C#N)=C/C(C)(C)N2CCN(C3COC3)CC2)C1] | (R,E)-2-(5-(4-amino-3-(2- fluoro-4-phenoxyphenyl)- 1H-pyrazolo[3,4- d]pyrimidin-1-yl)-3,3- dimethylpiperidine-1- carbonyl)-4-methyl-4-(4- (oxetan-3-yl)piperazin-1- yl)pent-2-enenitrile |
| 4-D | 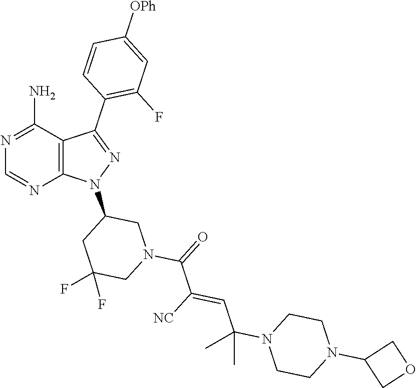 [SMILES: CC(C)(/C=C(\C#N)C(=O)N1C[C@H](n2nc(-c3ccc(Oc4ccccc4)cc3F)c3c(N)ncnc32)CC(F)(F)C1)N1CCN(C2COC2)CC1] | (R,E)-2-(5-(4-amino-3-(2- fluoro-4-phenoxyphenyl)- 1H-pyrazolo[3,4- d]pyrimidin-1-yl)-3,3- difluoropiperidine-1- carbonyl)-4-methyl-4-(4- (oxetan-3-yl)piperazin-1- yl)pent-2-enenitrile |
| 5-A | 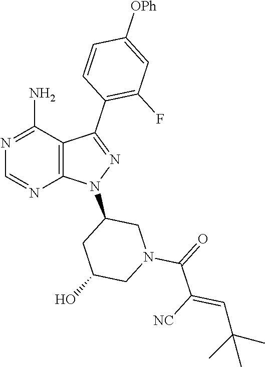 [SMILES: CC(C)(C)/C=C(\C#N)C(=O)N1C[C@H](O)C[C@@H](n2nc(-c3ccc(Oc4ccccc4)cc3F)c3c(N)ncnc32)C1] | (E)-2-((3R,5R)-3-(4-amino- 3-(2-fluoro-4- phenoxyphenyl)-1H- pyrazolo[3,4-d]pyrimidin-1- yl)-5-hydroxypiperidine-1- carbonyl)-4,4-dimethylpent- 2-enenitrile |
| 5-B | 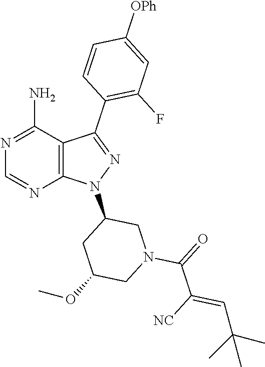 [SMILES: CO[C@@H]1C[C@@H](n2nc(-c3ccc(Oc4ccccc4)cc3F)c3c(N)ncnc32)CN(C(=O)/C(C#N)=C/C(C)(C)C)C1] | (E)-2-((3R,5R)-3-(4-amino- 3-(2-fluoro-4- phenoxyphenyl)-1H- pyrazolo[3,4-d]pyrimidin-1- yl)-5-methoxypiperidine-1- carbonyl)-4,4-dimethylpent- 2-enenitrile |
| 5-C | 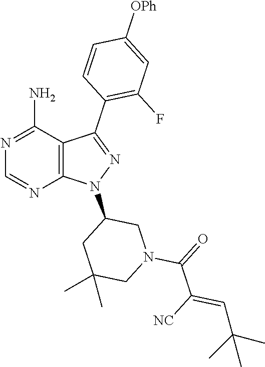 [SMILES: CC(C)(C)/C=C(\C#N)C(=O)N1C[C@H](n2nc(-c3ccc(Oc4ccccc4)cc3F)c3c(N)ncnc32)CC(C)(C)C1] | (E)-2-((3R,5R)-3-(4-amino- 3-(2-fluoro-4- phenoxyphenyl)-1H- pyrazolo[3,4-d]pyrimidin-1- yl)-5-methoxypiperidine-1- carbonyl)-4,4-dimethylpent- 2-enenitrile |
| 5-D | 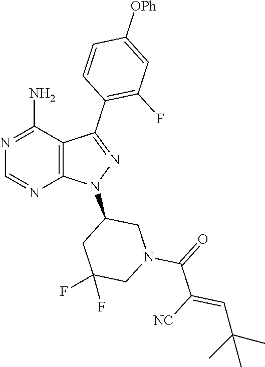 [SMILES: CC(C)(C)/C=C(\C#N)C(=O)N1C[C@H](n2nc(-c3ccc(Oc4ccccc4)cc3F)c3c(N)ncnc32)CC(F)(F)C1] | (R,E)-2-(5-(4-amino-3-(2- fluoro-4-phenoxyphenyl)- 1H-pyrazolo[3,4- d]pyrimidin-1-yl)-3,3- difluoropiperidine-1- carbonyl)-4,4-dimethylpent- 2-enenitrile |
| 6-A | 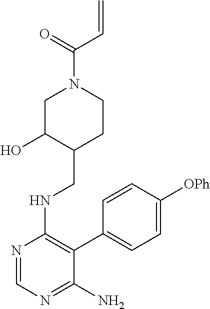 [SMILES: C=CC(=O)N1CCC(CNc2ncnc(N)c2-c2ccc(Oc3ccccc3)cc2)C(O)C1] | 1-(4-(((6-amino-5-(4- phenoxyphenyl)pyrimidin-4- yl)amino)methyl)-3- hydroxypiperidin-1-yl)prop- 2-en-1-one |
| 6-B | 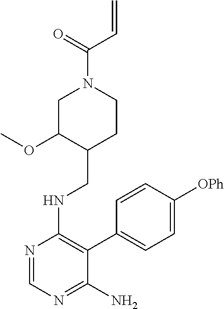 [SMILES: C=CC(=O)N1CCC(CNc2ncnc(N)c2-c2ccc(Oc3ccccc3)cc2)C(OC)C1] | 1-(4-(((6-amino-5-(4- phenoxyphenyl)pyrimidin-4- yl)amino)methyl)-3- methoxypiperidin-1-yl)prop- 2-en-1-one |
| 6-C | 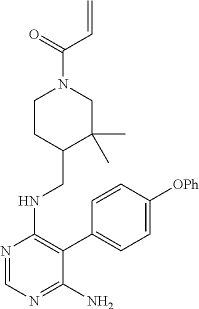 [SMILES: C=CC(=O)N1CCC(CNc2ncnc(N)c2-c2ccc(Oc3ccccc3)cc2)C(C)(C)C1] | 1-(4-(((6-amino-5-(4- phenoxyphenyl)pyrimidin-4- yl)amino)methyl)-3,3- dimethylpiperidin-1-yl)prop- 2-en-1-one |
| 6-D | 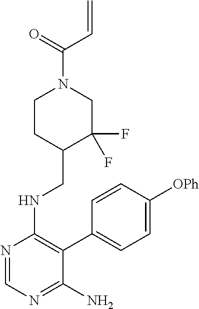 [SMILES: C=CC(=O)N1CCC(CNc2ncnc(N)c2-c2ccc(Oc3ccccc3)cc2)C(F)(F)C1] | 1-(4-(((6-amino-5-(4- phenoxyphenyl)pyrimidin-4- yl)amino)methyl)-3,3- dimethylpiperidin-1-yl)prop- 2-en-1-one |
| 7-A | 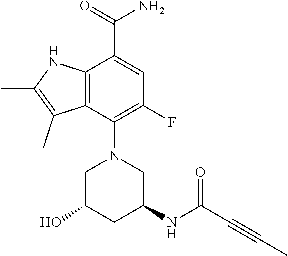 [SMILES: CC#CC(=O)N[C@H]1C[C@H](O)CN(c2c(F)cc(C(N)=O)c3[nH]c(C)c(C)c23)C1] | 4-((3S,5S)-3-(but-2- ynamido)-5- hydroxypiperidin-1-yl)-5- fluoro-2,3-dimethyl-1H- indole-7-carboxamide |
| 7-B | 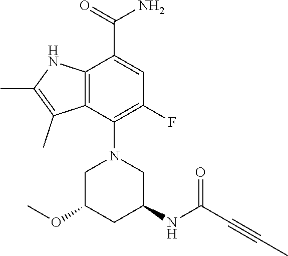 [SMILES: CC#CC(=O)N[C@H]1C[C@H](OC)CN(c2c(F)cc(C(N)=O)c3[nH]c(C)c(C)c23)C1] | 4-((3S,5S)-3-(but-2- ynamido)-5- methoxypiperidin-1-yl)-5- fluoro-2,3-dimethyl-1H- indole-7-carboxamide |
| 7-C | 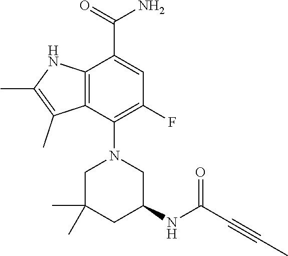 [SMILES: CC#CC(=O)N[C@@H]1CN(c2c(F)cc(C(N)=O)c3[nH]c(C)c(C)c23)CC(C)(C)C1] | (S)-4-(5-(but-2-ynamido)- 3,3-dimethylpiperidin-1-yl)- 5-fluoro-2,3-dimethyl-1H- indole-7-carboxamide |
| 7-D | 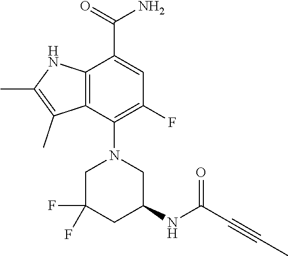 [SMILES: CC#CC(=O)N[C@@H]1CN(c2c(F)cc(C(N)=O)c3[nH]c(C)c(C)c23)CC(F)(F)C1] | (S)-4-(5-(but-2-ynamido)- 3,3-difluoropiperidin-1-yl)- 5-fluoro-2,3-dimethyl-1H- indole-7-carboxamide |
| 8-A | 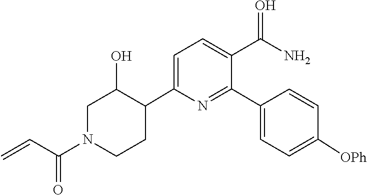 [SMILES: C=CC(=O)N1CCC(c2ccc(C(N)=O)c(-c3ccc(Oc4ccccc4)cc3)n2)C(O)C1] | 6-(1-acryloyl-3- hydroxypiperidin-4-yl)-2-(4- phenoxyphenyl)nicotinamide |
| 8-B | 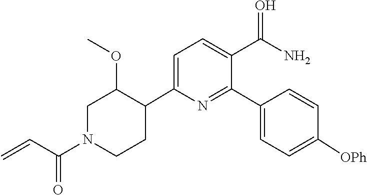 [SMILES: C=CC(=O)N1CCC(c2ccc(C(N)=O)c(-c3ccc(Oc4ccccc4)cc3)n2)C(OC)C1] | 6-(1-acryloyl-3- methoxypiperidin-4-yl)-2-(4- phenoxyphenyl)nicotinamide |
| 8-C | 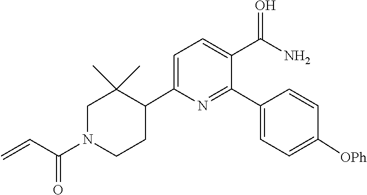 [SMILES: C=CC(=O)N1CCC(c2ccc(C(N)=O)c(-c3ccc(Oc4ccccc4)cc3)n2)C(C)(C)C1] | 6-(1-acryloyl-3,3- dimethylpiperidin-4-yl)-2- (4- phenoxyphenyl)nicotinamide |
| 8-D | 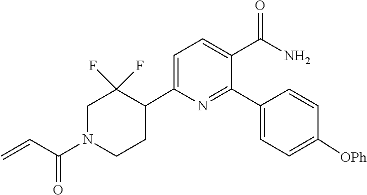 [SMILES: C=CC(=O)N1CCC(c2ccc(C(N)=O)c(-c3ccc(Oc4ccccc4)cc3)n2)C(F)(F)C1] | 6-(1-acryloyl-3,3- difluoropiperidin-4-yl)-2-(4- phenoxyphenyl)nicotinamide |
| 9-A & 9-A' | 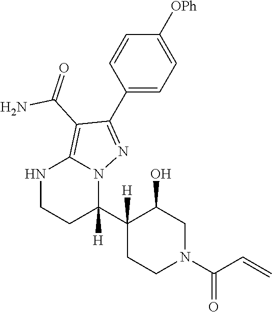 [SMILES: C=CC(=O)N1CC[C@H]([C@@H]2CCNc3c(C(N)=O)c(-c4ccc(Oc5ccccc5)cc4)nn32)[C@@H](O)C1] | (S)-7-((3R,4R)-1-acryloyl-3- hydroxypiperidin-4-yl)-2-(4- phenoxyphenyl)-4,5,6,7- tetrahydropyrazolo[1,5- a]pyrimidine-3-carboxamide & (S)-7-((3R,4S)-1-acryloyl-3- hydroxypiperidin-4-yl)-2-(4- phenoxyphenyl)-4,5,6,7- tetrahydropyrazolo[1,5- a]pyrimidine-3-carboxamide |
| | 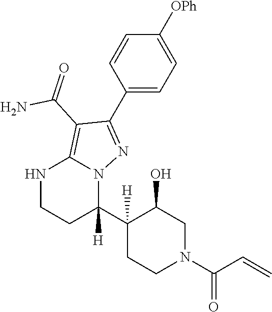 [SMILES: C=CC(=O)N1CC[C@@H]([C@@H]2CCNc3c(C(N)=O)c(-c4ccc(Oc5ccccc5)cc4)nn32)[C@@H](O)C1] | |
| 9-B | 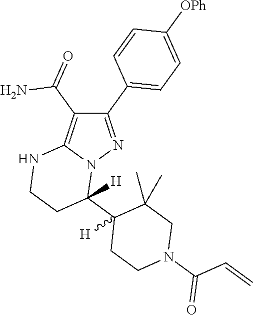 [SMILES: C=CC(=O)N1CCC([C@@H]2CCNc3c(C(N)=O)c(-c4ccc(Oc5ccccc5)cc4)nn32)C(C)(C)C1] | ((7S)-7-(1-acryloyl-3,3- dimethylpiperidin-4-)-2- (4-phenoxyphenyl)-4,5,6,7- tetrahydropyrazolo[ 1,5- a]pyrimidine-3-carboxamide |
| 9-C | 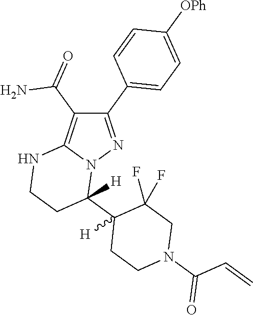 [SMILES: C=CC(=O)N1CCC([C@@H]2CCNc3c(C(N)=O)c(-c4ccc(Oc5ccccc5)cc4)nn32)C(F)(F)C1] | (7S)-7-(1-acryloyl-3,3- difluoropiperidin-4-yl)-2-(4- phenoxyphenyl)-4,5,6,7- tetrahydropyrazolo[1,5- a]pyrimidine-3-carboxamide |

or a pharmaceutically acceptable salt thereof.

It is understood that in the present description, combinations of substituents and/or variables of the depicted formulae are permissible only if such contributions result in stable compounds.

Furthermore, all compounds of Formula (1)-(9) that exist in free base or acid form can be converted to their pharmaceutically acceptable salts by treatment with the appropriate inorganic or organic base or acid by methods known to one skilled in the art. Salts of the compounds of Formula (1)-(9) can be converted to their free base or acid form by standard techniques.

In some embodiments, the present disclosure relates to a composition comprising a pharmaceutically acceptable excipient and at least one compound chosen from any one of the compounds described herein.

In some embodiments, the present disclosure relates to a method of treating a disease or disorder mediated by BTK comprising administering to a patient in need thereof an effective amount of a compound chosen from any one of the compounds described herein, or administering a composition as described herein.

The compounds described herein can be made using conventional organic syntheses and commercially available starting materials, or the methods provided herein. It should be noted that one skilled in the art would know how to modify the procedures set forth in the illustrative schemes and examples to arrive at the desired products.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

Boc: tert-butyloxycarbonyl
DAST: diethylaminosulfur trifluoride
DBU: 1,8-diazabicyclo[5.4.0]undec-7-ene
DEAD: diethyl azodicarboxylate
DIPEA: diisopropylethylamine
DMF: dimethylformamide
DMSO: dimethyl sulfoxide
EDCI: 1-ethyl-3-(3-dimethylaminopropyl)carbodiimide
EtOH: ethanol
HATU: 1-[bis(dimethylamino)methylene]-1H-1,2,3-triazolo[4,5-b]pyridinium 3-oxide
hexafluorophosphate, Hexafluorophosphate Azabenzotriazole Tetramethyl Uronium
HOBt: hydroxybenzotriazole
i-PrOH: isopropyl alcohol
mCPBA: meta-chloroperoxybenzoic acid
MeOH: methanol
Pd(dppf)Cl₂: [1,1′-bis(diphenylphosphino)ferrocene]palladium(II) dichloride
SFC: supercritical-fluid chromatography
TBTU: 2-(1H-benzotriazole-1-yl)-1,1,3,3-tetramethylaminium tetrafluoroborate
TEA: triethylamine
TEMPO: (2,2,6,6-tetramethylpiperidin-1-yl)oxyl
TFA: trifluoroacetic acid
THF: tetrahydrofuran
tol: toluene

### Example S1. 1-((3R,5R)-3-(4-amino-3-(4-phenoxyphenyl)-1H-pyrazolo[3,4-d]pyrimidin-1-yl)-5-hydroxypiperidin-1-yl)prop-2-en-1-one (1-A)

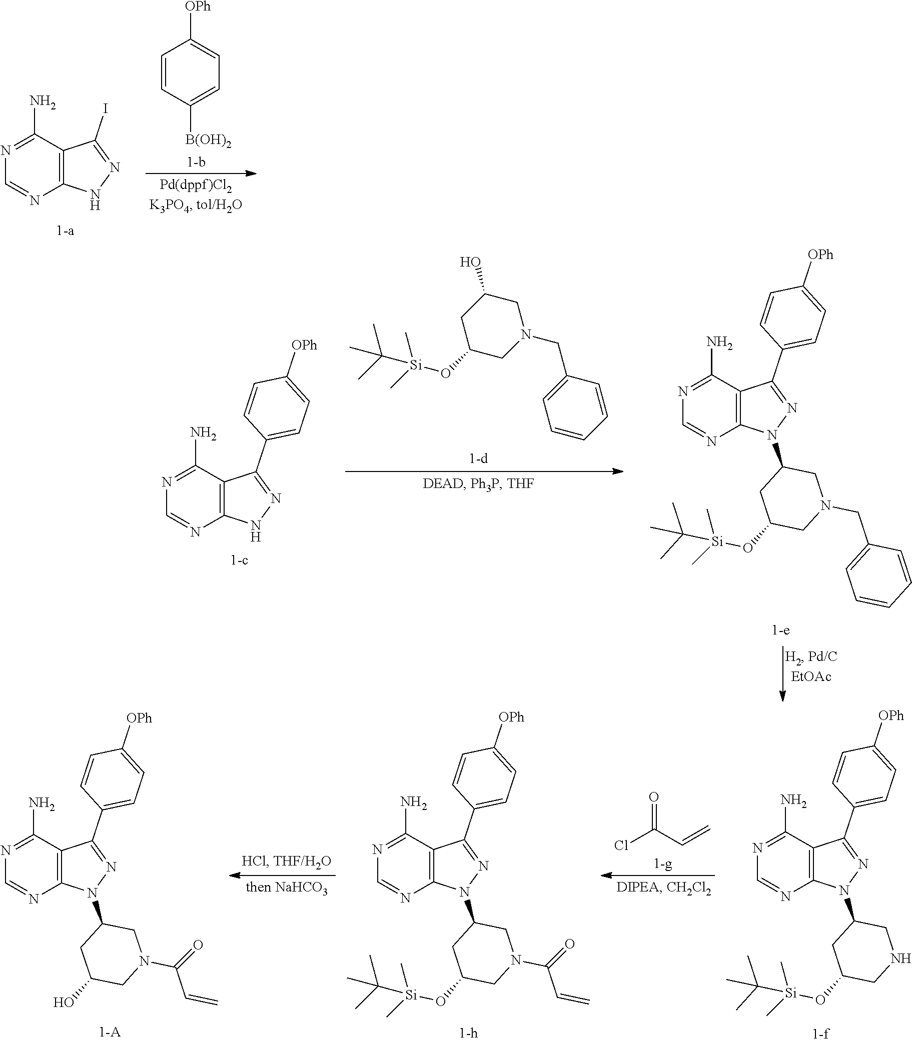

To a solution of 3-iodo-1H-pyrazolo[3,4-d]pyrimidin-4-amine (1-a) in toluene and water is added (4-phenoxyphenyl)boronic acid (1-b), Pd(dppf)Cl₂, and K₃PO₄. Stirring the reaction solution at 90-100° C. yields 3-(4-phenoxyphenyl)-1H-pyrazolo[3,4-d]pyrimidin-4-amine (1-c). 1-c is dissolved in THF and is cooled to 0° C., where (3S,5R)-1-benzyl-5-((tert-butyldimethylsilyl)oxy)piperidin-3-ol (1-d), diethyl azodicarboxylate, and triphenylphosphine is added. The reaction solution is stirred at room temperature to yield 1-((3R,5R)-1-benzyl-5-((tert-butyldimethylsilyl)oxy)piperidin-3-yl)-3-(4-phenoxyphenyl)-1H-pyrazolo[3,4-d]pyrimidin-4-amine (1-e). 1-e is dissolved in ethyl acetate and hydrogenated with H₂ and Pd/C to yield 1-((3R,5R)-5-((tert-butyldimethylsilyl)oxy)piperidin-3-yl)-3-(4-phenoxyphenyl)-1H-pyrazolo[3,4-d]pyrimidin-4-amine (1-f). 1-f is reacted with acryloyl chloride (1-g) in dichloromethane in the presence of a N,N-diisopropylethylamine base to yield 1-((3R,5R)-3-(4-amino-3-(4-phenoxyphenyl)-1H-pyrazolo[3,4-d]pyrimidin-1-yl)-5-((tert-butyldimethylsilyl)oxy)piperidin-1-yl)prop-2-en-1-one (1-h). Deprotection of the tert-butyldimethylsilyl-protected alcohol of 1-h in acidic conditions yields 1-((3R,5R)-3-(4-amino-3-(4-phenoxyphenyl)-1H-pyrazolo[3,4-d]pyrimidin-1-yl)-5-hydroxypiperidin-1-yl)prop-2-en-1-one (1-A).

### Example S2. 1-((3R,5R)-3-(4-amino-3-(4-phenoxyphenyl)-1H-pyrazolo[3,4-d]pyrimidin-1-yl)-5-methoxypiperidin-1-yl)prop-2-en-1-one (1-B)

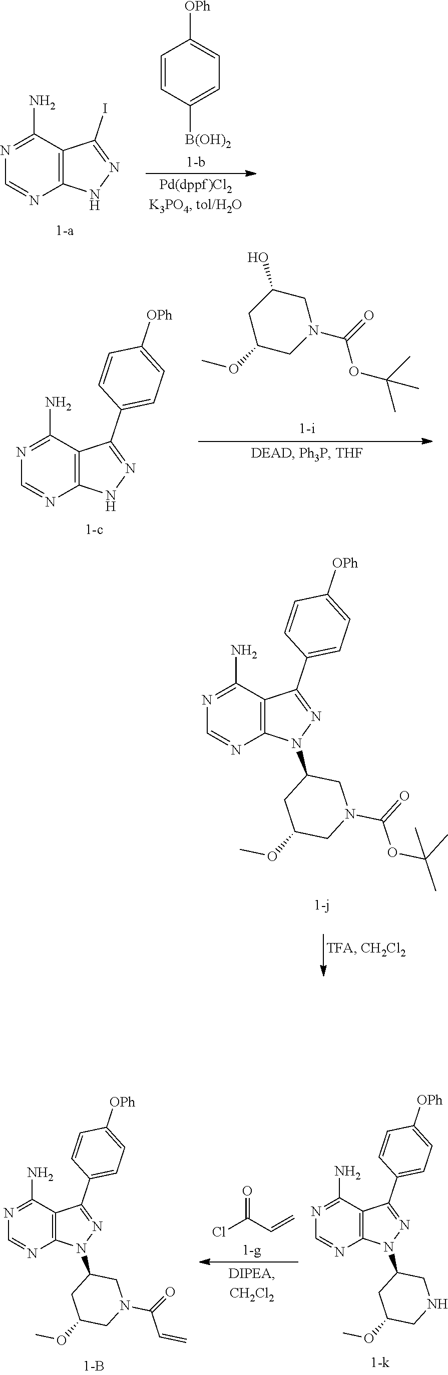

To a solution of 3-iodo-1H-pyrazolo[3,4-d]pyrimidin-4-amine (1-a) in toluene and water is added (4-phenoxyphenyl)boronic acid (1-b), Pd(dppf)Cl₂, and K₃PO₄. Stirring the reaction solution at 90-100° C. yields 3-(4-phenoxyphenyl)-1H-pyrazolo[3,4-d]pyrimidin-4-amine (1-c). 1-c is dissolved in THE and is cooled to 0° C., where tert-butyl (3S,5R)-3-hydroxy-5-methoxypiperidine-1-carboxylate (1-i), diethyl azodicarboxylate, and triphenylphosphine is added. The reaction solution is stirred at room temperature to yield tert-butyl (3R,5R)-3-(4-amino-3-(4-phenoxyphenyl)-1H-pyrazolo[3,4-d]pyrimidin-1-yl)-5-methoxypiperidine-1-carboxylate (1-j). Deprotection of the Boc-protected amine of 1-j in acidic conditions yields 1-((3R,5R)-5-methoxypiperidin-3-yl)-3-(4-phenoxyphenyl)-1H-pyrazolo[3,4-d]pyrimidin-4-amine (1-k), which is then reacted with acryloyl chloride (1-g) in dichloromethane in the presence of a N,N-diisopropylethylamine base to yield 1-((3R,5R)-3-(4-amino-3-(4-phenoxyphenyl)-1H-pyrazolo[3,4-d]pyrimidin-1-yl)-5-methoxypiperidin-1-yl)prop-2-en-1-one (1-B).

### Example S3. (R)-1-(5-(4-amino-3-(4-phenoxyphenyl)-1H-pyrazolo[3,4-d]pyrimidin-1-yl)-3,3-dimethylpiperidin-1-yl)prop-2-en-1-one (1-C)

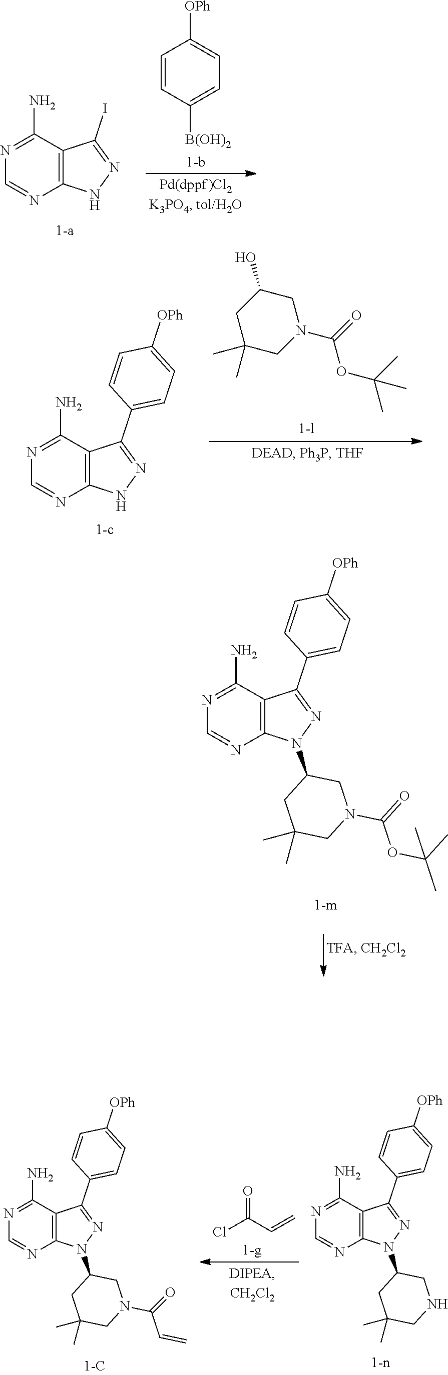

To a solution of 3-iodo-1H-pyrazolo[3,4-d]pyrimidin-4-amine (1-a) in toluene and water is added (4-phenoxyphenyl)boronic acid (1-b), Pd(dppf)Cl₂, and K₃PO₄. Stirring the reaction solution at 90-100° C. yields 3-(4-phenoxyphenyl)-1H-pyrazolo[3,4-d]pyrimidin-4-amine (1-c). 1-c is dissolved in THE and is cooled to 0° C., where tert-butyl (S)-5-hydroxy-3,3-dimethylpiperidine-1-carboxylate (1-1), diethyl azodicarboxylate, and triphenylphosphine is added. The reaction solution is stirred at room temperature to yield tert-butyl (R)-5-(4-amino-3-(4-phenoxyphenyl)-1H-pyrazolo[3,4-d]pyrimidin-1-yl)-3,3-dimethylpiperidine-1-carboxylate (1-m). Deprotection of the Boc-protected amine of 1-m in acidic conditions yields (R)-1-(5,5-dimethylpiperidin-3-yl)-3-(4-phenoxyphenyl)-1H-pyrazolo[3,4-d]pyrimidin-4-amine (1-n), which is then reacted with acryloyl chloride (1-g) in dichloromethane in the presence of a N,N-diisopropylethylamine base to yield (R)-1-(5-(4-amino-3-(4-phenoxyphenyl)-1H-pyrazolo[3,4-d]pyrimidin-1-yl)-3,3-dimethylpiperidin-1-yl)prop-2-en-1-one (1-C).

### Example S4. (R)-1-(5-(4-amino-3-(4-phenoxyphenyl)-1H-pyrazolo[3,4-d]pyrimidin-1-yl)-3,3-difluoropiperidin-1-yl)prop-2-en-1-one (1-D)

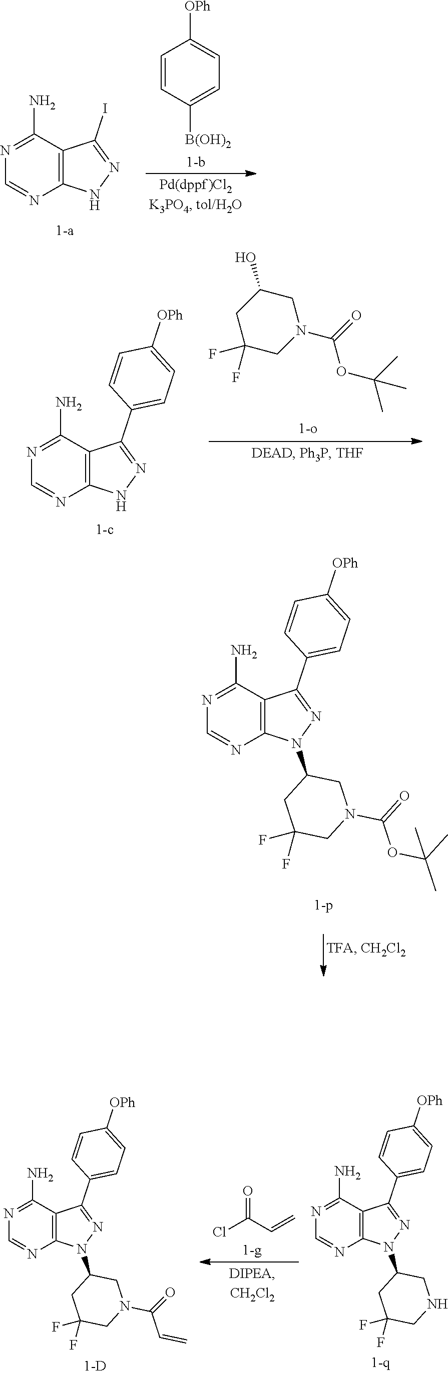

To a solution of 3-iodo-1H-pyrazolo[3,4-d]pyrimidin-4-amine (1-a) in toluene and water is added (4-phenoxyphenyl)boronic acid (1-b), Pd(dppf)Cl₂, and K₃PO₄. Stirring the reaction solution at 90-100° C. yields 3-(4-phenoxyphenyl)-1H-pyrazolo[3,4-d]pyrimidin-4-amine (1-c). 1-c is dissolved in THE and is cooled to 0° C., where tert-butyl (S)-3,3-difluoro-5-hydroxypiperidine-1-carboxylate (1-o), diethyl azodicarboxylate, and triphenylphosphine is added. The reaction solution is stirred at room temperature to yield tert-butyl (R)-5-(4-amino-3-(4-phenoxyphenyl)-1H-pyrazolo[3,4-d]pyrimidin-1-yl)-3,3-difluoropiperidine-1-carboxylate (1-p). Deprotection of the Boc-protected amine of 1-p in acidic conditions yields (R)-1-(5,5-difluoropiperidin-3-yl)-3-(4-phenoxyphenyl)-1H-pyrazolo[3,4-d]pyrimidin-4-amine (1-q), which is then reacted with acryloyl chloride (1-g) in dichloromethane in the presence of a N,N-diisopropylethylamine base to yield (R)-1-(5-(4-amino-3-(4-phenoxyphenyl)-1H-pyrazolo[3,4-d]pyrimidin-1-yl)-3,3-difluoropiperidin-1-yl)prop-2-en-1-one (1-D).

### Example S5. 4-amino-1-((3S,5R)-1-(2-fluoroacryloyl)-5-hydroxypiperidin-3-yl)-3-(4-phenoxyphenyl)-1,3-dihydro-2H-imidazo[4,5-c]pyridin-2-one (2-A)

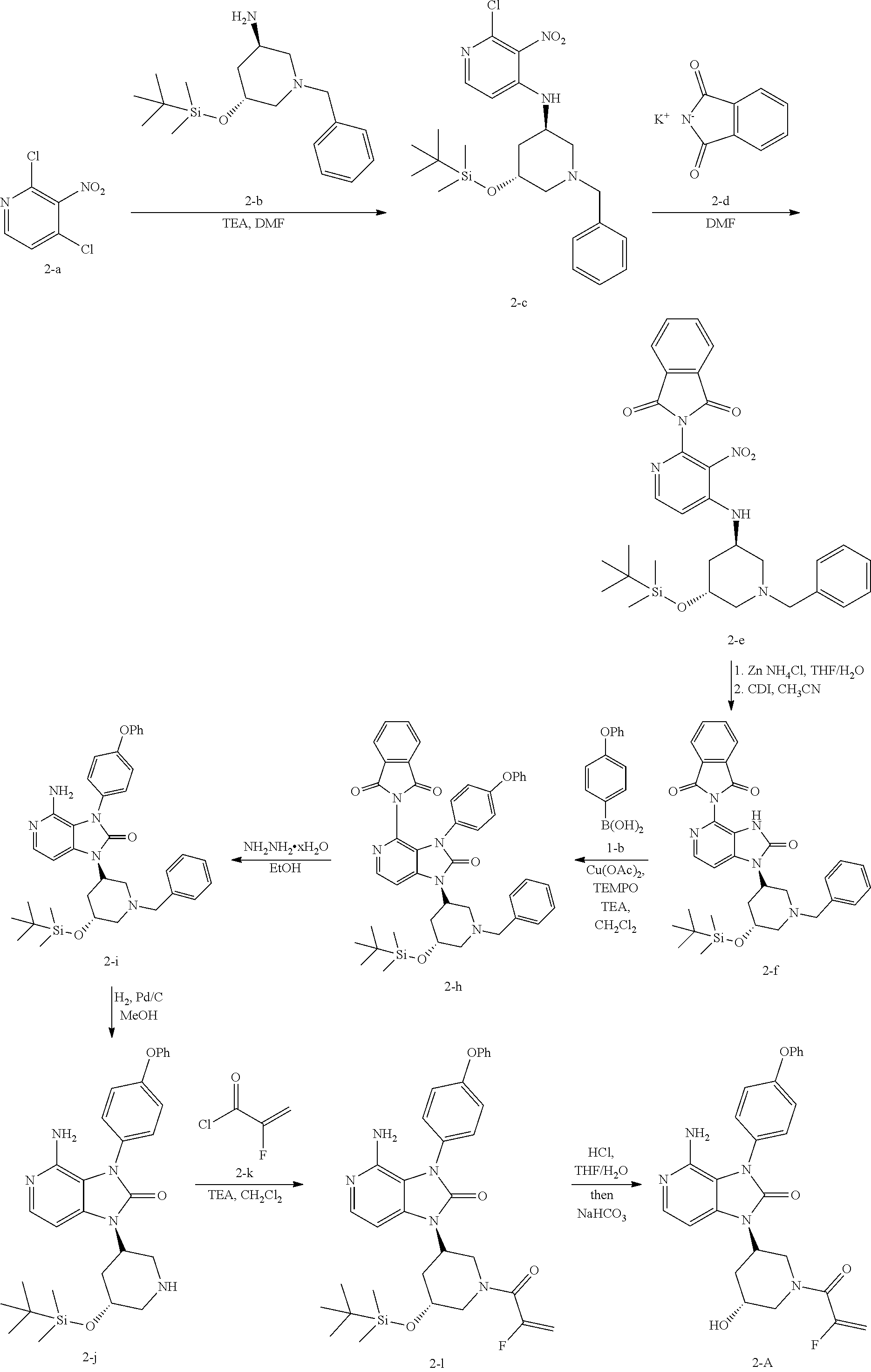

To a solution of 2,4-dichloro-3-nitropyridine (2-a) and triethylamine in DMF is added (3R,5R)-1-benzyl-5-((tert-butyldimethylsilyl)oxy)piperidin-3-amine (2-b). The reaction solution is stirred at room temperature to yield N-((3R,5R)-1-benzyl-5-((tert-butyldimethylsilyl)oxy)piperidin-3-yl)-2-chloro-3-nitropyridin-4-amine (2-c), which is dissolved in DMF and reacted with potassium 1,3-dioxoisoindolin-2-ide (2-d) to yield 2-(4-(((3R,5R)-1-benzyl-5-((tert-butyldimethylsilyl)oxy)piperidin-3-yl)amino)-3-nitropyridin-2-yl)isoindoline-1,3-dione (2-e). 2-e is reacted with zinc and NH₄Cl in THF/H₂O to reduce its nitro group, followed by reaction with carbonyldiimidazole in acetonitrile to form 2-(1-((3S,5R)-1-benzyl-5-((tert-butyldimethylsilyl)oxy)piperidin-3-yl)-2-oxo-2,3-dihydro-1H-imidazo[4,5-c]pyridin-4-yl)isoindoline-1,3-dione (2-f). 2-f undergoes a cross-coupling reaction with (4-phenoxyphenyl)boronic acid (1-b) with Cu(OAc)₂, TEMPO, and triethylamine in dichloromethane to yield 2-(1-((3S,5R)-1-benzyl-5-((tert-butyldimethylsilyl)oxy)piperidin-3-yl)-2-oxo-3-(4-phenoxyphenyl)-2,3-dihydro-1H-imidazo[4,5-c]pyridin-4-yl)isoindoline-1,3-dione (2-h). The phthalimide-protected amine of 2-h is deprotected by reacting with hydrazine in ethanol to yield 4-amino-1-((3S,5R)-1-benzyl-5-((tert-butyldimethylsilyl)oxy)piperidin-3-yl)-3-(4-phenoxyphenyl)-1,3-dihydro-2H-imidazo[4,5-c]pyridin-2-one (2-i), and the remaining benzyl-protected amine is deprotected by reacting 2-i with H₂ and Pd/C in MeOH to yield 4-amino-1-((3S,5R)-5-((tert-butyldimethylsilyl)oxy)piperidin-3-yl)-3-(4-phenoxyphenyl)-1,3-dihydro-2H-imidazo[4,5-c]pyridin-2-one (2-j). 2-j is reacted with 2-fluoroacryloyl chloride (2-k) in the presence of a triethylamine base in dichloromethane to yield 4-amino-1-((3S,5R)-5-((tert-butyldimethylsilyl)oxy)-1-(2-fluoroacryloyl)piperidin-3-yl)-3-(4-phenoxyphenyl)-1,3-dihydro-2H-imidazo[4,5-c]pyridin-2-one (2-1). The tert-butyldimethylsilyl-protected alcohol of 2-1 is deprotected in acidic conditions to yield 4-amino-1-((3S,5R)-1-(2-fluoroacryloyl)-5-hydroxypiperidin-3-yl)-3-(4-phenoxyphenyl)-1,3-dihydro-2H-imidazo[4,5-c]pyridin-2-one (2-A).

### Example S6. 4-amino-1-((3S,5R)-1-(2-fluoroacryloyl)-5-methoxypiperidin-3-yl)-3-(4-phenoxyphenyl)-1,3-dihydro-2H-imidazo[4,5-c]pyridin-2-one (2-B)

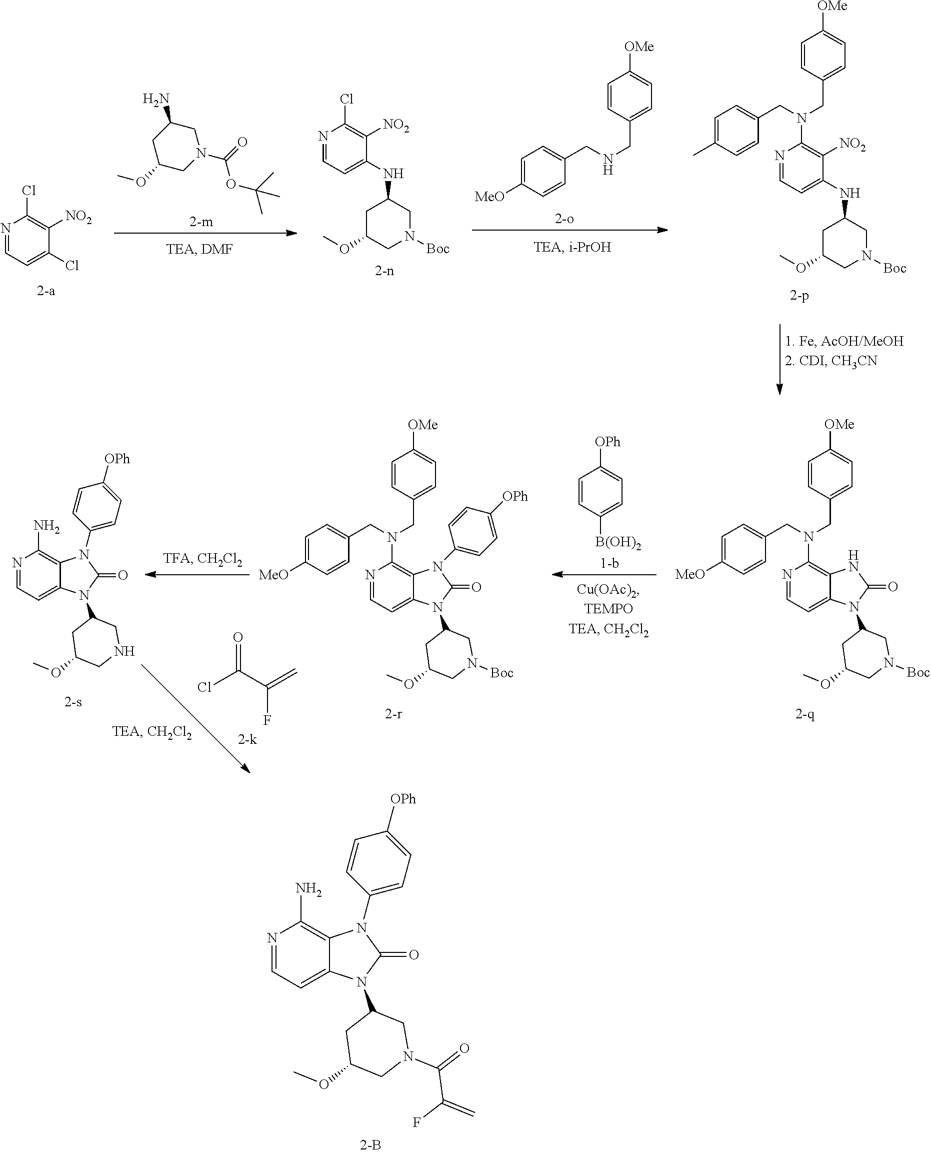

To a solution of 2,4-dichloro-3-nitropyridine (2-a) and triethylamine in DMF is added tert-butyl (3R,5R)-3-amino-5-methoxypiperidine-1-carboxylate (2-m). The reaction solution is stirred at room temperature to yield tert-butyl (3R,5R)-3-((2-chloro-3-nitropyridin-4-yl)amino)-5-methoxypiperidine-1-carboxylate (2-n), which is is reacted with bis(4-methoxybenzyl)amine (2-0) with a triethylamine base in isopropyl alcohol to yield tert-butyl (3R,5R)-3-methoxy-5-((2-((4-methoxybenzyl)(4-methylbenzyl)amino)-3-nitropyridin-4-yl)amino)piperidine-1-carboxylate (2-p). 2-p is reacted with Fe in AcOH/MeOH to reduce its nitro group, followed by reaction with carbonyldiimidazole in acetonitrile to form tert-butyl (3S,5R)-3-(4-(bis(4-methoxybenzyl)amino)-2-oxo-2,3-dihydro-1H-imidazo[4,5-c]pyridin-1-yl)-5-methoxypiperidine-1-carboxylate (2-q). 2-q undergoes a cross-coupling reaction with (4-phenoxyphenyl)boronic acid (1-b) with Cu(OAc)₂, TEMPO, and triethylamine in dichloromethane to yield tert-butyl (3S,5R)-3-(4-(bis(4-methoxybenzyl)amino)-2-oxo-3-(4-phenoxyphenyl)-2,3-dihydro-1H-imidazo[4,5-c]pyridin-1-yl)-5-methoxypiperidine-1-carboxylate (2-r). The Boc-protected amine of 2-r is deprotected in acidic conditions to yield 4-amino-1-((3S,5R)-5-methoxypiperidin-3-yl)-3-(4-phenoxyphenyl)-1,3-dihydro-2H-imidazo[4,5-c]pyridin-2-one (2-s), and the free amine of 2-s is reacted with 2-fluoroacryloyl chloride (2-k) in the presence of a triethylamine base in dichloromethane to yield 4-amino-1-((3S,5R)-1-(2-fluoroacryloyl)-5-methoxypiperidin-3-yl)-3-(4-phenoxyphenyl)-1,3-dihydro-2H-imidazo[4,5-c]pyridin-2-one (2-B).

### Example S7. (S)-4-amino-1-(1-(2-fluoroacryloyl)-5,5-dimethylpiperidin-3-yl)-3-(4-phenoxyphenyl)-1,3-dihydro-2H-imidazo[4,5-c]pyridin-2-one (2-C)

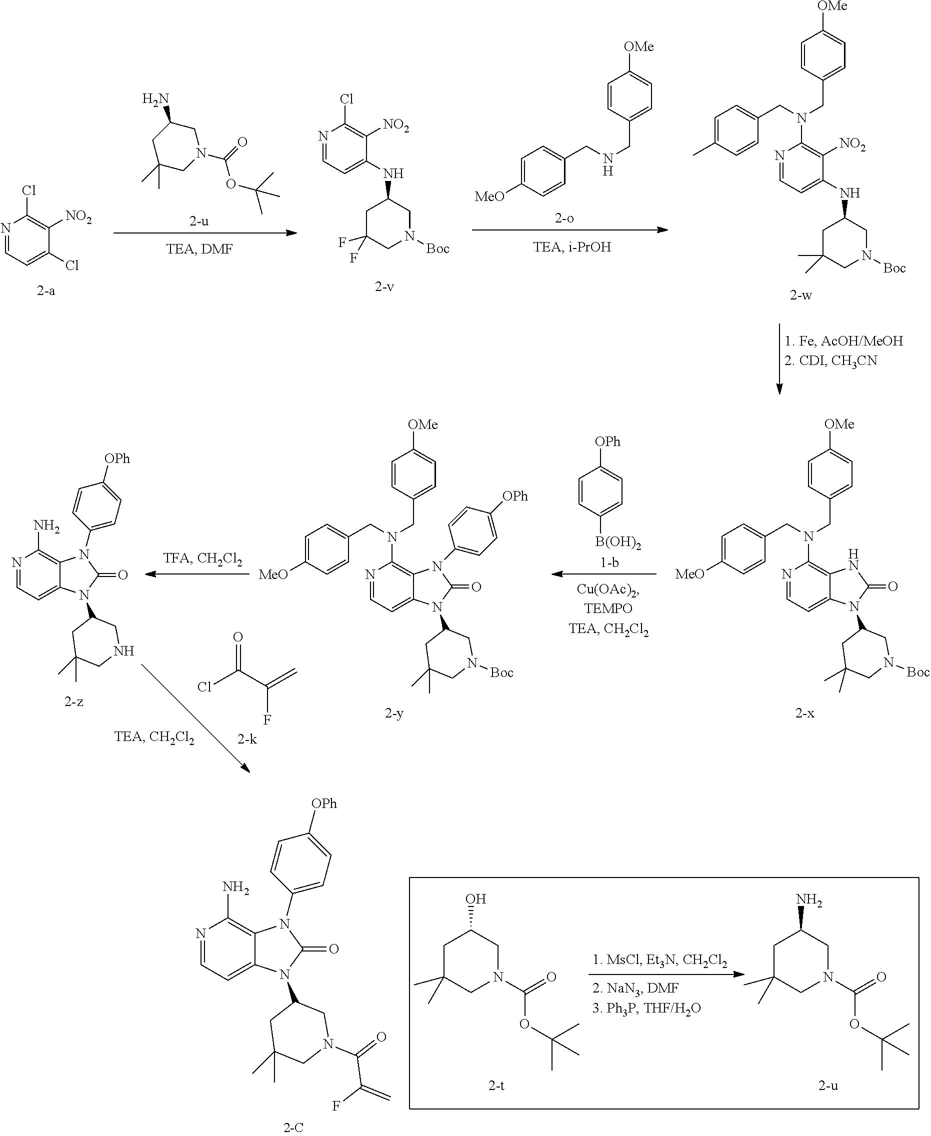

The alcohol of tert-butyl (S)-5-hydroxy-3,3-dimethylpiperidine-1-carboxylate (2-t) is activated with mesyl chloride in the presence of a triethylamine base in dichloromethane, which is subsequently converted to an amine through subsequent reaction with NaN₃ in DMF and triphenylphosphine in THF/H₂O to yield tert-butyl (R)-5-amino-3,3-dimethylpiperidine-1-carboxylate (2-u). 2-u is added to a solution of 2,4-dichloro-3-nitropyridine (2-a) and triethylamine in DMF and stirred at room temperature to yield tert-butyl (R)-5-((2-chloro-3-nitropyridin-4-yl)amino)-3,3-difluoropiperidine-1-carboxylate (2-v), which is is reacted with bis(4-methoxybenzyl)amine (2-0) with a triethylamine base in isopropyl alcohol to yield tert-butyl (R)-5-((2-((4-methoxybenzyl)(4-methylbenzyl)amino)-3-nitropyridin-4-yl)amino)-3,3-dimethylpiperidine-1-carboxylate (2-w). 2-w is reacted with Fe in AcOH/MeOH to reduce its nitro group, followed by reaction with carbonyldiimidazole in acetonitrile to form tert-butyl (S)-5-(4-(bis(4-methoxybenzyl)amino)-2-oxo-2,3-dihydro-1H-imidazo[4,5-c]pyridin-1-yl)-3,3-dimethylpiperidine-1-carboxylate (2-x). 2-x undergoes a cross-coupling reaction with (4-phenoxyphenyl)boronic acid (1-b) with Cu(OAc)₂, TEMPO, and triethylamine in dichloromethane to yield tert-butyl (S)-5-(4-(bis(4-methoxybenzyl)amino)-2-oxo-3-(4-phenoxyphenyl)-2,3-dihydro-1H-imidazo[4,5-c]pyridin-1-yl)-3,3-dimethylpiperidine-1-carboxylate (2-y). The Boc-protected amine of 2-y is deprotected in acidic conditions to yield (S)-4-amino-1-(5,5-dimethylpiperidin-3-yl)-3-(4-phenoxyphenyl)-1,3-dihydro-2H-imidazo[4,5-c]pyridin-2-one (2-z), and the free amine of 2-z is reacted with 2-fluoroacryloyl chloride (2-k) in the presence of a triethylamine base in dichloromethane to yield (S)-4-amino-1-(1-(2-fluoroacryloyl)-5,5-dimethylpiperidin-3-yl)-3-(4-phenoxyphenyl)-1,3-dihydro-2H-imidazo[4,5-c]pyridin-2-one (2-C).

### Example S8. (S)-4-amino-1-(5,5-difluoro-1-(2-fluoroacryloyl)piperidin-3-yl)-3-(4-phenoxyphenyl)-1,3-dihydro-2H-imidazo[4,5-c]pyridin-2-one (2-D)

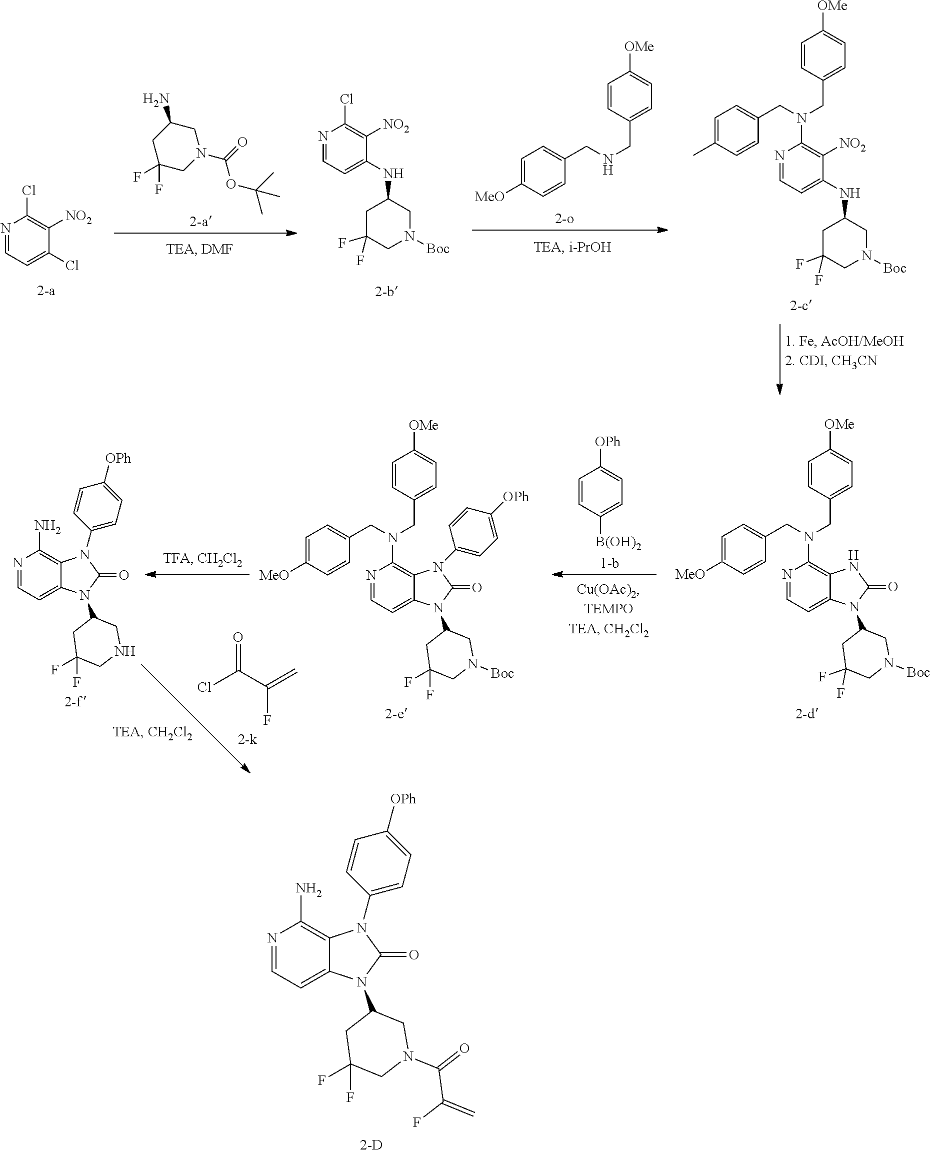

To a solution of 2,4-dichloro-3-nitropyridine (2-a) and triethylamine in DMF is added tert-butyl (R)-5-amino-3,3-difluoropiperidine-1-carboxylate (2-a′). The reaction solution is stirred at room temperature to yield tert-butyl (R)-5-((2-chloro-3-nitropyridin-4-yl)amino)-3,3-difluoropiperidine-1-carboxylate (2-b′), which is reacted with bis(4-methoxybenzyl)amine (2-0) with a triethylamine base in isopropyl alcohol to yield tert-butyl (R)-3,3-difluoro-5-((2-((4-methoxybenzyl)(4-methylbenzyl)amino)-3-nitropyridin-4-yl)amino)piperidine-1-carboxylate (2-c′). 2-c′ is reacted with Fe in AcOH/MeOH to reduce its nitro group, followed by reaction with carbonyldiimidazole in acetonitrile to form tert-butyl (S)-5-(4-(bis(4-methoxybenzyl)amino)-2-oxo-2,3-dihydro-1H-imidazo[4,5-c]pyridin-1-yl)-3,3-difluoropiperidine-1-carboxylate (2-d′). 2-d′ undergoes a cross-coupling reaction with (4-phenoxyphenyl)boronic acid (1-b) with Cu(OAc)₂, TEMPO, and triethylamine in dichloromethane to yield tert-butyl (S)-5-(4-(bis(4-methoxybenzyl)amino)-2-oxo-3-(4-phenoxyphenyl)-2,3-dihydro-1H-imidazo[4,5-c]pyridin-1-yl)-3,3-difluoropiperidine-1-carboxylate (2-e′). The Boc-protected amine of 2-e′ is deprotected in acidic conditions to yield (S)-4-amino-1-(5,5-difluoropiperidin-3-yl)-3-(4-phenoxyphenyl)-1,3-dihydro-2H-imidazo[4,5-c]pyridin-2-one (2-f′), and the free amine of 2-f is reacted with 2-fluoroacryloyl chloride (2-k) in the presence of a triethylamine base in dichloromethane to yield (S)-4-amino-1-(5,5-difluoro-1-(2-fluoroacryloyl)piperidin-3-yl)-3-(4-phenoxyphenyl)-1,3-dihydro-2H-imidazo[4,5-c]pyridin-2-one (2-D).

### Example S9. 1-((3S,5R)-1-acryloyl-5-hydroxypiperidin-3-yl)-4-amino-3-(4-phenoxyphenyl)-1,3-dihydro-2H-imidazo[4,5-c]pyridin-2-one (3-A)

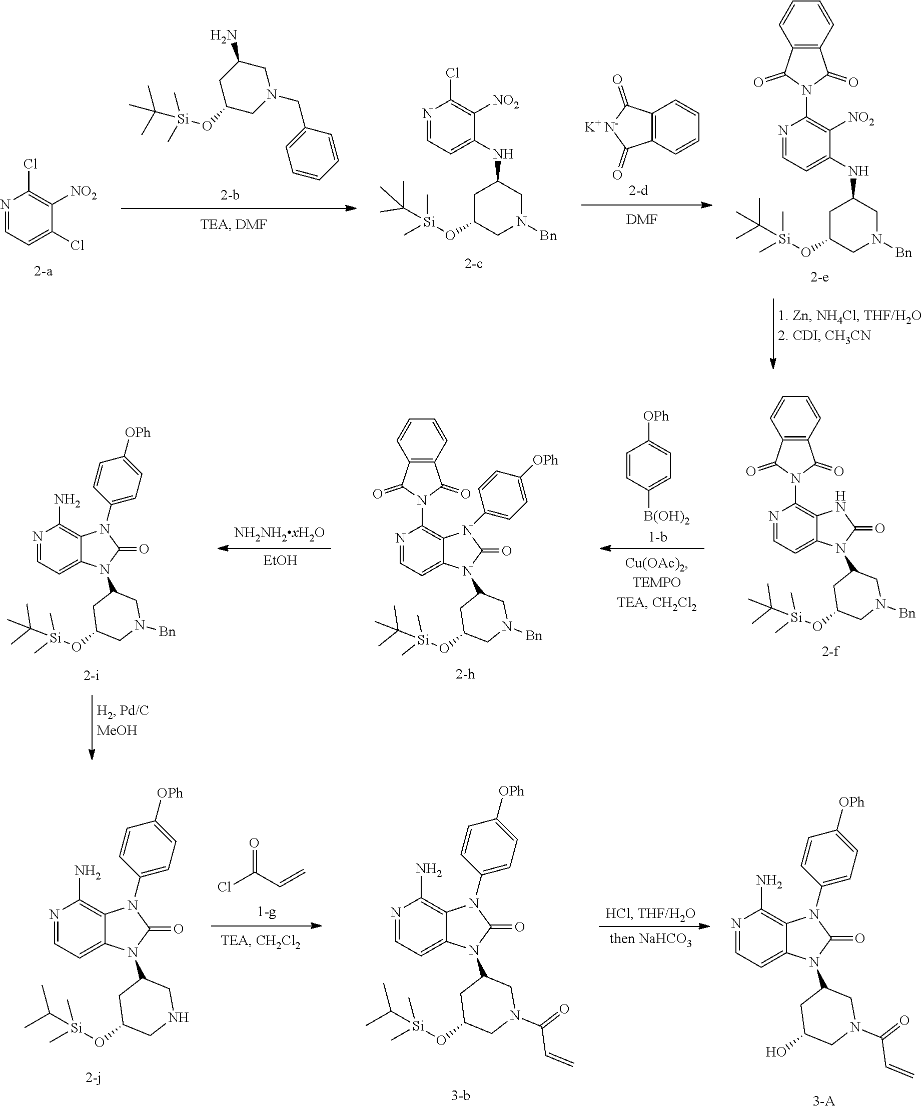

To a solution of 2,4-dichloro-3-nitropyridine (2-a) and triethylamine in DMF is added (3R,5R)-1-benzyl-5-((tert-butyldimethylsilyl)oxy)piperidin-3-amine (2-b). The reaction solution is stirred at room temperature to yield N-((3R,5R)-1-benzyl-5-((tert-butyldimethylsilyl)oxy)piperidin-3-yl)-2-chloro-3-nitropyridin-4-amine (2-c), which is dissolved in DMF and reacted with potassium 1,3-dioxoisoindolin-2-ide (2-d) to yield 2-(4-(((3R,5R)-1-benzyl-5-((tert-butyldimethylsilyl)oxy)piperidin-3-yl)amino)-3-nitropyridin-2-yl)isoindoline-1,3-dione (2-e). 2-e is reacted with zinc and NH₄Cl in THF/H₂O to reduce its nitro group, followed by reaction with carbonyldiimidazole in acetonitrile to form 2-(1-((3S,5R)-1-benzyl-5-((tert-butyldimethylsilyl)oxy)piperidin-3-yl)-2-oxo-2,3-dihydro-1H-imidazo[4,5-c]pyridin-4-yl)isoindoline-1,3-dione (2-f). 2-f undergoes a cross-coupling reaction with (4-phenoxyphenyl)boronic acid (1-b) with Cu(OAc)₂, TEMPO, and triethylamine in dichloromethane to yield 2-(1-((3S,5R)-1-benzyl-5-((tert-butyldimethylsilyl)oxy)piperidin-3-yl)-2-oxo-3-(4-phenoxyphenyl)-2,3-dihydro-1H-imidazo[4,5-c]pyridin-4-yl)isoindoline-1,3-dione (2-h). The phthalimide-protected amine of 2-h is deprotected by reacting with hydrazine in ethanol to yield 4-amino-1-((3S,5R)-1-benzyl-5-((tert-butyldimethylsilyl)oxy)piperidin-3-yl)-3-(4-phenoxyphenyl)-1,3-dihydro-2H-imidazo[4,5-c]pyridin-2-one (2-i), and the remaining benzyl-protected amine is deprotected by reacting 2-i with H₂ and Pd/C in MeOH to yield 4-amino-1-((3S,5R)-5-((tert-butyldimethylsilyl)oxy)piperidin-3-yl)-3-(4-phenoxyphenyl)-1,3-dihydro-2H-imidazo[4,5-c]pyridin-2-one (2-j). 2-j is reacted with acryloyl chloride (1-g) in the presence of a triethylamine base in dichloromethane to yield 1-((3S,5R)-1-acryloyl-5-((tert-butyldimethylsilyl)oxy)piperidin-3-yl)-4-amino-3-(4-phenoxyphenyl)-1,3-dihydro-2H-imidazo[4,5-c]pyridin-2-one (3-b). The tert-butyldimethylsilyl-protected alcohol of 3-b is deprotected in acidic conditions to yield 1-((3S,5R)-1-acryloyl-5-hydroxypiperidin-3-yl)-4-amino-3-(4-phenoxyphenyl)-1,3-dihydro-2H-imidazo[4,5-c]pyridin-2-one (3-A).

### Example S10. 1-((3S,5R)-1-acryloyl-5-methoxypiperidin-3-yl)-4-amino-3-(4-phenoxyphenyl)-1,3-dihydro-2H-imidazo[4,5-c]pyridin-2-one (3-B)

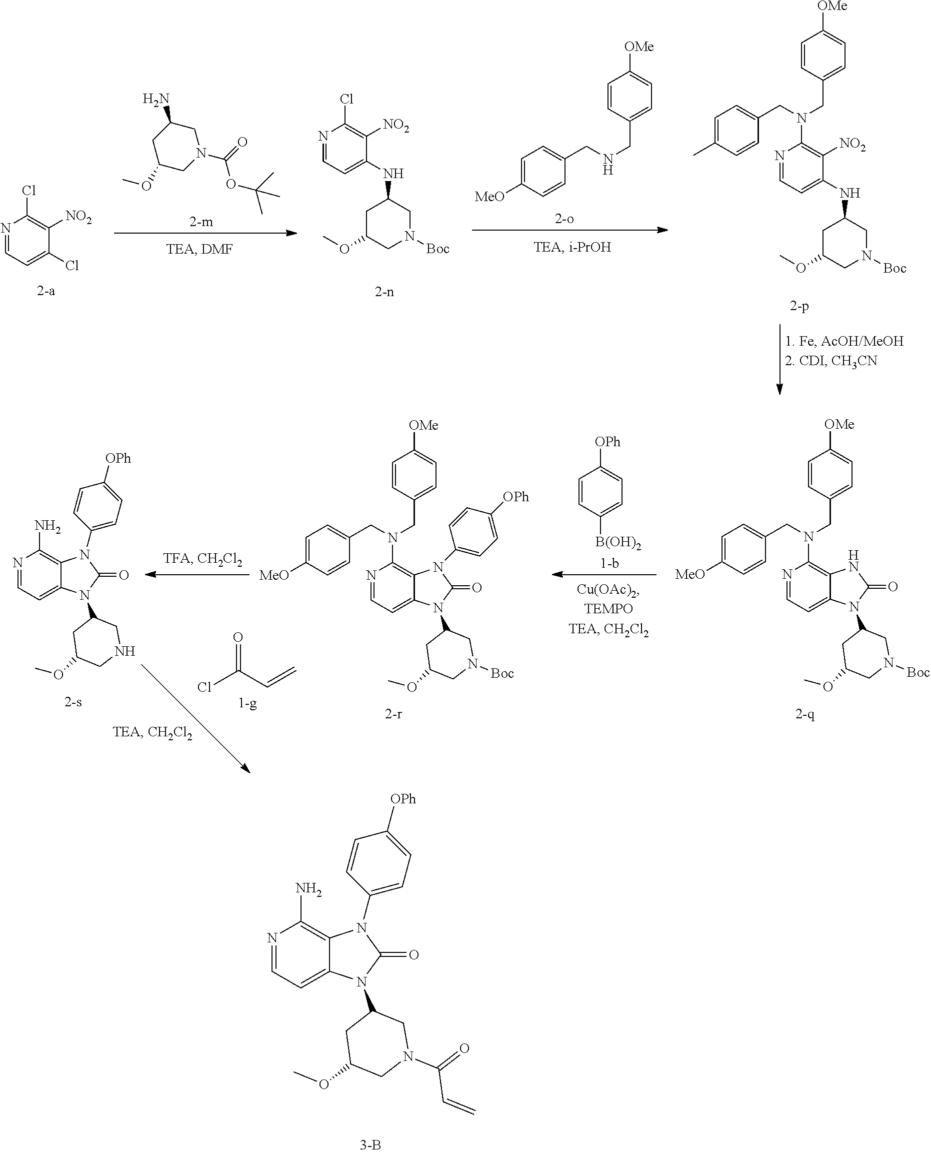

To a solution of 2,4-dichloro-3-nitropyridine (2-a) and triethylamine in DMF is added tert-butyl (3R,5R)-3-amino-5-methoxypiperidine-1-carboxylate (2-m). The reaction solution is stirred at room temperature to yield tert-butyl (3R,5R)-3-((2-chloro-3-nitropyridin-4-yl)amino)-5-methoxypiperidine-1-carboxylate (2-n), which is is reacted with bis(4-methoxybenzyl)amine (2-0) with a triethylamine base in isopropyl alcohol to yield tert-butyl (3R,5R)-3-methoxy-5-((2-((4-methoxybenzyl)(4-methylbenzyl)amino)-3-nitropyridin-4-yl)amino)piperidine-1-carboxylate (2-p). 2-p is reacted with Fe in AcOH/MeOH to reduce its nitro group, followed by reaction with carbonyldiimidazole in acetonitrile to form tert-butyl (3S,5R)-3-(4-(bis(4-methoxybenzyl)amino)-2-oxo-2,3-dihydro-1H-imidazo[4,5-c]pyridin-1-yl)-5-methoxypiperidine-1-carboxylate (2-q). 2-q undergoes a cross-coupling reaction with (4-phenoxyphenyl)boronic acid (1-b) with Cu(OAc)₂, TEMPO, and triethylamine in dichloromethane to yield tert-butyl (3S,5R)-3-(4-(bis(4-methoxybenzyl)amino)-2-oxo-3-(4-phenoxyphenyl)-2,3-dihydro-1H-imidazo[4,5-c]pyridin-1-yl)-5-methoxypiperidine-1-carboxylate (2-r). The Boc-protected amine of 2-r is deprotected in acidic conditions to yield 4-amino-1-((3S,5R)-5-methoxypiperidin-3-yl)-3-(4-phenoxyphenyl)-1,3-dihydro-2H-imidazo[4,5-c]pyridin-2-one (2-s), and the free amine of 2-s is reacted with acryloyl chloride (1-g) in the presence of a triethylamine base in dichloromethane to yield 1-((3S,5R)-1-acryloyl-5-methoxypiperidin-3-yl)-4-amino-3-(4-phenoxyphenyl)-1,3-dihydro-2H-imidazo[4,5-c]pyridin-2-one (3-B).

### Example S11. (S)-4-amino-1-(1-(2-fluoroacryloyl)-5,5-dimethylpiperidin-3-yl)-3-(4-phenoxyphenyl)-1,3-dihydro-2H-imidazo[4,5-c]pyridin-2-one (3-C)

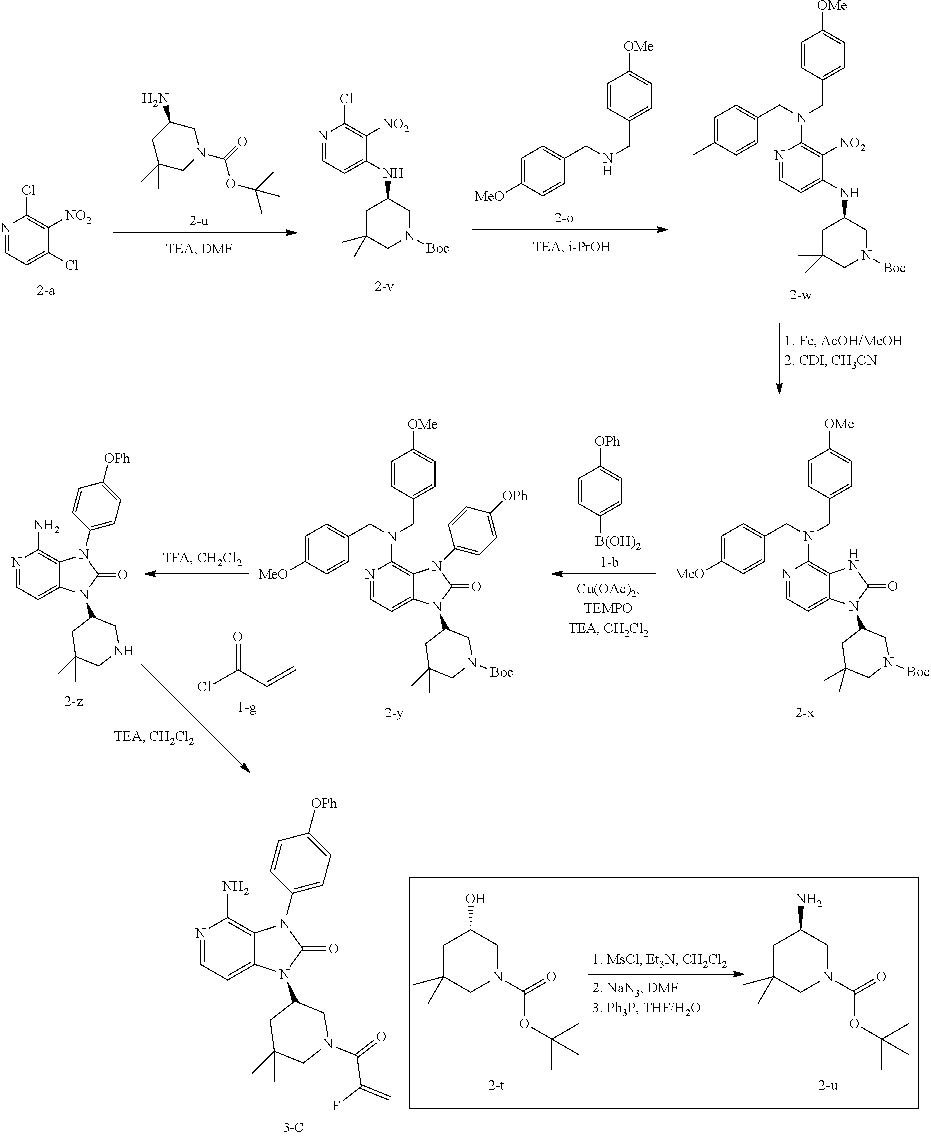

The alcohol of tert-butyl (S)-5-hydroxy-3,3-dimethylpiperidine-1-carboxylate (2-t) is activated with mesyl chloride in the presence of a triethylamine base in dichloromethane, which is subsequently converted to an amine through subsequent reaction with NaN₃ in DMF and triphenylphosphine in THF/H₂O to yield tert-butyl (R)-5-amino-3,3-dimethylpiperidine-1-carboxylate (2-u). 2-u is added to a solution of 2,4-dichloro-3-nitropyridine (2-a) and triethylamine in DMF and stirred at room temperature to yield tert-butyl (R)-5-((2-chloro-3-nitropyridin-4-yl)amino)-3,3-difluoropiperidine-1-carboxylate (2-v), which is is reacted with bis(4-methoxybenzyl)amine (2-0) with a triethylamine base in isopropyl alcohol to yield tert-butyl (R)-5-((2-((4-methoxybenzyl)(4-methylbenzyl)amino)-3-nitropyridin-4-yl)amino)-3,3-dimethylpiperidine-1-carboxylate (2-w). 2-w is reacted with Fe in AcOH/MeOH to reduce its nitro group, followed by reaction with carbonyldiimidazole in acetonitrile to form tert-butyl (S)-5-(4-(bis(4-methoxybenzyl)amino)-2-oxo-2,3-dihydro-1H-imidazo[4,5-c]pyridin-1-yl)-3,3-dimethylpiperidine-1-carboxylate (2-x). 2-x undergoes a cross-coupling reaction with (4-phenoxyphenyl)boronic acid (1-b) with Cu(OAc)₂, TEMPO, and triethylamine in dichloromethane to yield tert-butyl (S)-5-(4-(bis(4-methoxybenzyl)amino)-2-oxo-3-(4-phenoxyphenyl)-2,3-dihydro-1H-imidazo[4,5-c]pyridin-1-yl)-3,3-dimethylpiperidine-1-carboxylate (2-y). The Boc-protected amine of 2-y is deprotected in acidic conditions to yield (S)-4-amino-1-(5,5-dimethylpiperidin-3-yl)-3-(4-phenoxyphenyl)-1,3-dihydro-2H-imidazo[4,5-c]pyridin-2-one (2-z), and the free amine of 2-z is reacted with acryloyl chloride (1-g) in the presence of a triethylamine base in dichloromethane to yield (S)-4-amino-1-(1-(2-fluoroacryloyl)-5,5-dimethylpiperidin-3-yl)-3-(4-phenoxyphenyl)-1,3-dihydro-2H-imidazo[4,5-c]pyridin-2-one (3-C).

### Example S12. (S)-4-amino-1-(5,5-difluoro-1-(2-fluoroacryloyl)piperidin-3-yl)-3-(4-phenoxyphenyl)-1,3-dihydro-2H-imidazo[4,5-c]pyridin-2-one (3-D)

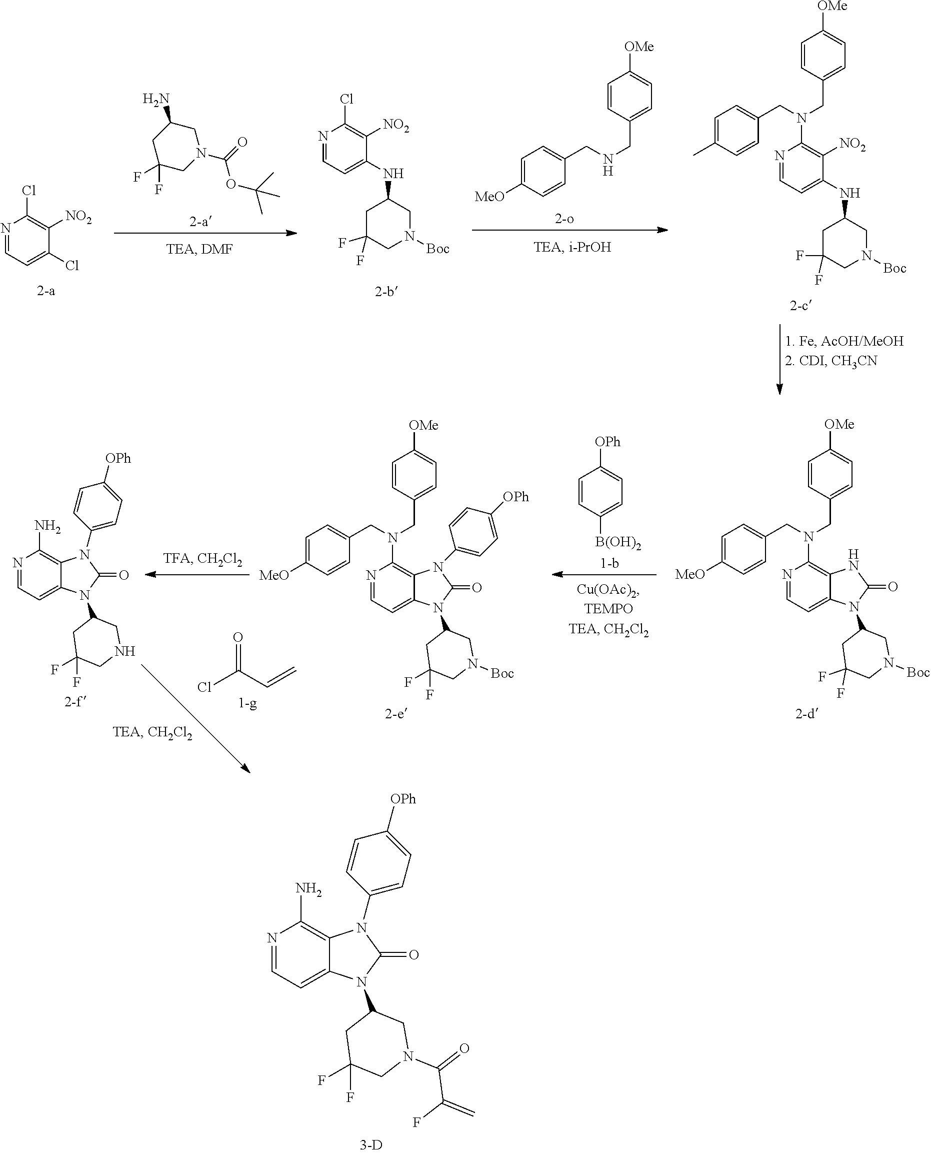

To a solution of 2,4-dichloro-3-nitropyridine (2-a) and triethylamine in DMF is added tert-butyl (R)-5-amino-3,3-difluoropiperidine-1-carboxylate (2-a′). The reaction solution is stirred at room temperature to yield tert-butyl (R)-5-((2-chloro-3-nitropyridin-4-yl)amino)-3,3-difluoropiperidine-1-carboxylate (2-b′), which is reacted with bis(4-methoxybenzyl)amine (2-0) with a triethylamine base in isopropyl alcohol to yield tert-butyl (R)-3,3-difluoro-5-((2-((4-methoxybenzyl)(4-methylbenzyl)amino)-3-nitropyridin-4-yl)amino)piperidine-1-carboxylate (2-c′). 2-c′ is reacted with Fe in AcOH/MeOH to reduce its nitro group, followed by reaction with carbonyldiimidazole in acetonitrile to form tert-butyl (S)-5-(4-(bis(4-methoxybenzyl)amino)-2-oxo-2,3-dihydro-1H-imidazo[4,5-c]pyridin-1-yl)-3,3-difluoropiperidine-1-carboxylate (2-d′). 2-d′ undergoes a cross-coupling reaction with (4-phenoxyphenyl)boronic acid (1-b) with Cu(OAc)₂, TEMPO, and triethylamine in dichloromethane to yield tert-butyl (S)-5-(4-(bis(4-methoxybenzyl)amino)-2-oxo-3-(4-phenoxyphenyl)-2,3-dihydro-1H-imidazo[4,5-c]pyridin-1-yl)-3,3-difluoropiperidine-1-carboxylate (2-e′). The Boc-protected amine of 2-e′ is deprotected in acidic conditions to yield (S)-4-amino-1-(5,5-difluoropiperidin-3-yl)-3-(4-phenoxyphenyl)-1,3-dihydro-2H-imidazo[4,5-c]pyridin-2-one (2-f′), and the free amine of 2-f is reacted with acryloyl chloride (1-g) in the presence of a triethylamine base in dichloromethane to yield (S)-4-amino-1-(5,5-difluoro-1-(2-fluoroacryloyl)piperidin-3-yl)-3-(4-phenoxyphenyl)-1,3-dihydro-2H-imidazo[4,5-c]pyridin-2-one (3-D).

### Example S13. (E)-2-((3R,5R)-3-(4-amino-3-(2-fluoro-4-phenoxyphenyl)-1H-pyrazolo[3,4-d]pyrimidin-1-yl)-5-hydroxypiperidine-1-carbonyl)-4-methyl-4-(4-(oxetan-3-yl)piperazin-1-yl)pent-2-enenitrile (4-A)

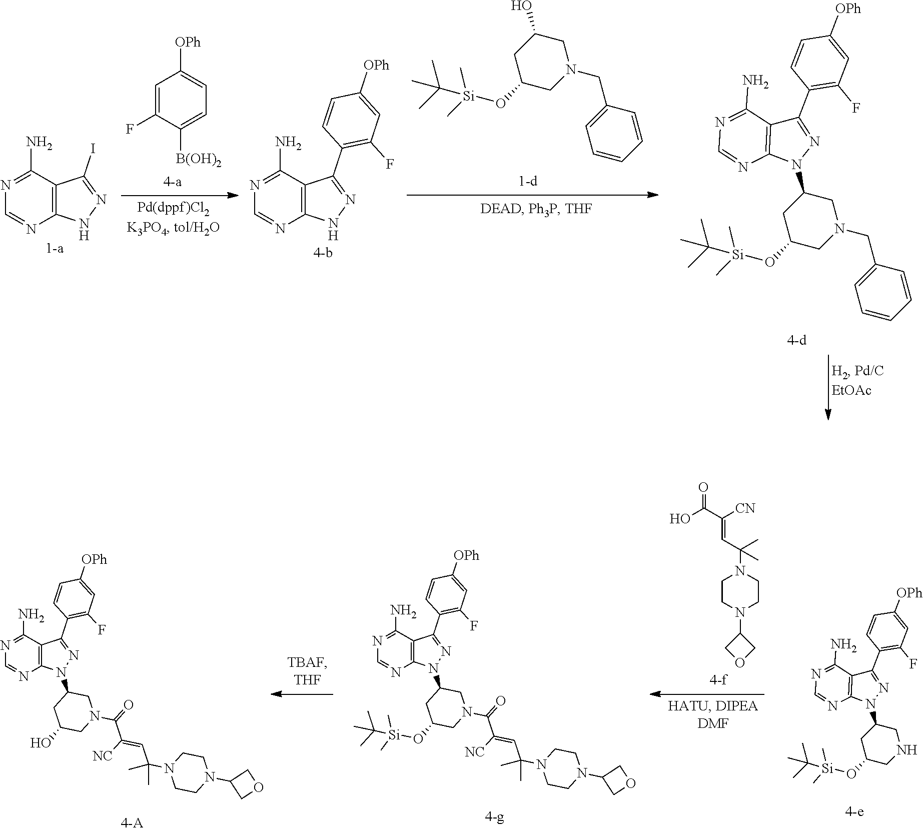

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where (3S,5R)-1-benzyl-5-((tert-butyldimethylsilyl)oxy)piperidin-3-ol (1-d), diethyl azodicarboxylate, and triphenylphosphine is added. The reaction solution is stirred at room temperature to yield 1-((3R,5R)-1-benzyl-5-((tert-butyldimethylsilyl)oxy)piperidin-3-yl)-3-(2-fluoro-4-phenoxyphenyl)-1H-pyrazolo[3,4-d]pyrimidin-4-amine (4-d). 4-d is dissolved in ethyl acetate and hydrogenated with H₂ and Pd/C to yield 1-((3R,5R)-5-((tert-butyldimethylsilyl)oxy)piperidin-3-yl)-3-(2-fluoro-4-phenoxyphenyl)-1H-pyrazolo[3,4-d]pyrimidin-4-amine (4-e). 4-e is reacted with (E)-2-cyano-4-methyl-4-(4-(oxetan-3-yl)piperazin-1-yl)pent-2-enoic acid (4-f) in DMF in the presence of HATU and a diisopropylethylamine base base to yield (E)-2-((3R,5R)-3-(4-amino-3-(2-fluoro-4-phenoxyphenyl)-1H-pyrazolo[3,4-d]pyrimidin-1-yl)-5-((tert-butyldimethylsilyl)oxy)piperidine-1-carbonyl)-4-methyl-4-(4-(oxetan-3-yl)piperazin-1-yl)pent-2-enenitrile (4-g). Deprotection of the tert-butyldimethylsilyl-protected alcohol of 4-g with tetrabutylammonium fluoride in THE yields (E)-2-((3R,5R)-3-(4-amino-3-(2-fluoro-4-phenoxyphenyl)-1H-pyrazolo[3,4-d]pyrimidin-1-yl)-5-hydroxypiperidine-1-carbonyl)-4-methyl-4-(4-(oxetan-3-yl)piperazin-1-yl)pent-2-enenitrile (4-A).

### Example S14. (E)-2-((3R,5R)-3-(4-amino-3-(2-fluoro-4-phenoxyphenyl)-1H-pyrazolo[3,4-d]pyrimidin-1-yl)-5-methoxypiperidine-1-carbonyl)-4-methyl-4-(4-(oxetan-3-yl)piperazin-1-yl)pent-2-enenitrile (4-B)

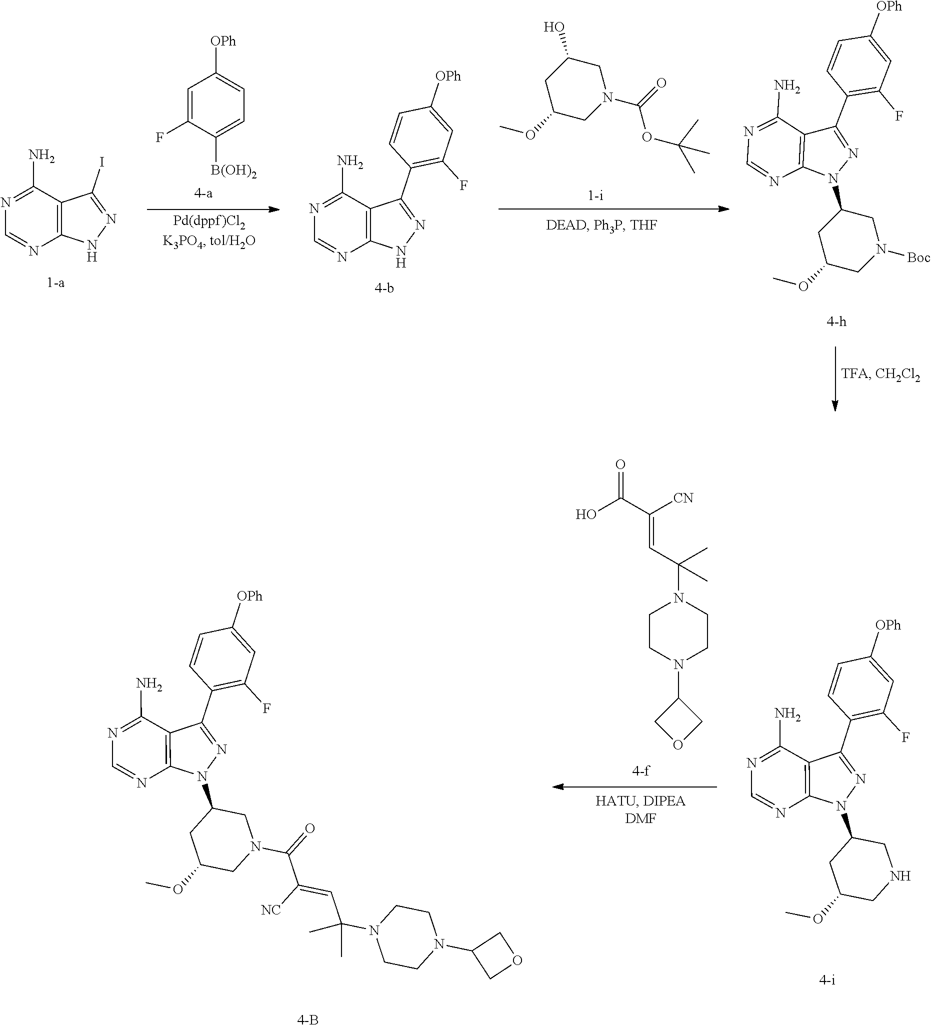

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (3S,5R)-3-hydroxy-5-methoxypiperidine-1-carboxylate (1-i), diethyl azodicarboxylate, and triphenylphosphine is added. The reaction solution is stirred at room temperature to yield tert-butyl (3R,5R)-3-(4-amino-3-(2-fluoro-4-phenoxyphenyl)-1H-pyrazolo[3,4-d]pyrimidin-1-yl)-5-methoxypiperidine-1-carboxylate (4-h). The Boc-protected amine of 4-h is deprotected in acidic conditions to yield 3-(2-fluoro-4-phenoxyphenyl)-1-((3R,5R)-5-methoxypiperidin-3-yl)-1H-pyrazolo[3,4-d]pyrimidin-4-amine (4-i), and the free amine of 4-i is reacted with (E)-2-cyano-4-methyl-4-(4-(oxetan-3-yl)piperazin-1-yl)pent-2-enoic acid (4-f) in DMF in the presence of HATU and a diisopropylethylamine base base to yield (E)-2-((3R,5R)-3-(4-amino-3-(2-fluoro-4-phenoxyphenyl)-1H-pyrazolo[3,4-d]pyrimidin-1-yl)-5-methoxypiperidine-1-carbonyl)-4-methyl-4-(4-(oxetan-3-yl)piperazin-1-yl)pent-2-enenitrile (4-B).

### Example S15. (R,E)-2-(5-(4-amino-3-(2-fluoro-4-phenoxyphenyl)-1H-pyrazolo[3,4-d]pyrimidin-1-yl)-3,3-dimethylpiperidine-1-carbonyl)-4-methyl-4-(4-(oxetan-3-yl)piperazin-1-yl)pent-2-enenitrile (4-C)

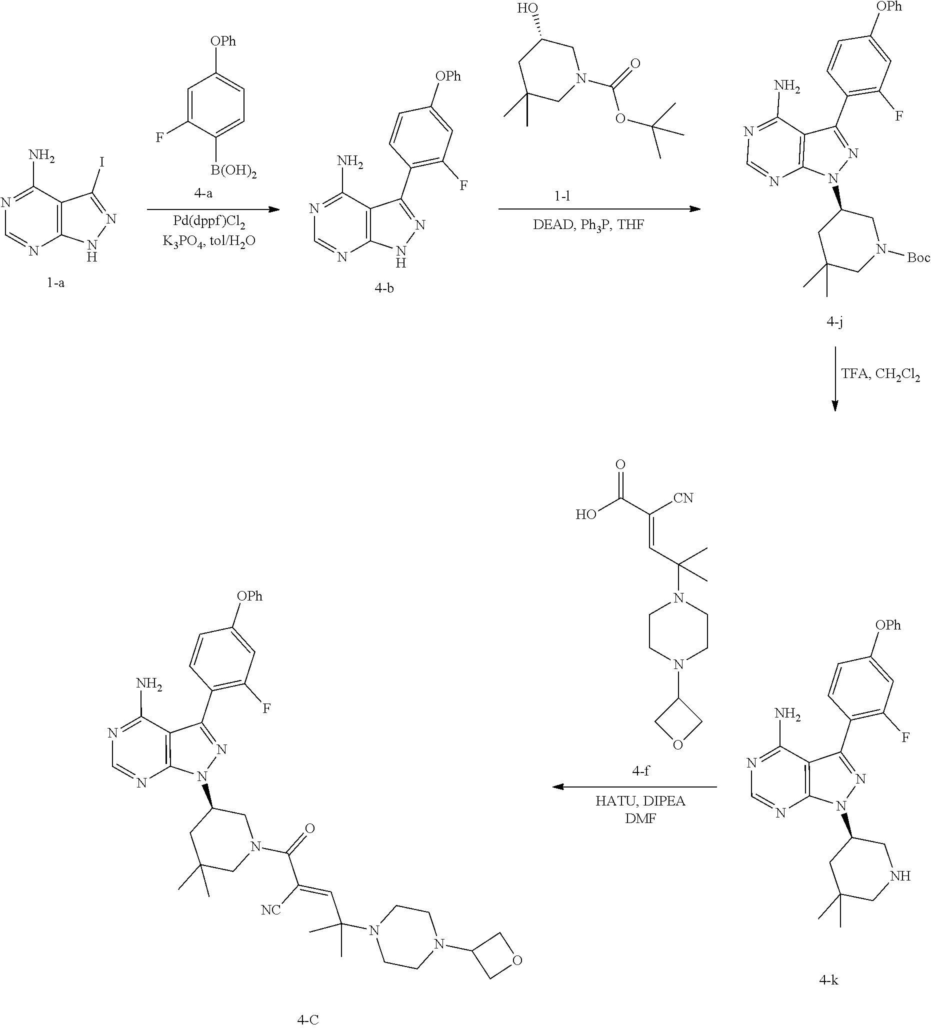

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (S)-5-hydroxy-3,3-dimethylpiperidine-1-carboxylate (1-l), diethyl azodicarboxylate, and triphenylphosphine is added. The reaction solution is stirred at room temperature to yield tert-butyl (R)-5-(4-amino-3-(2-fluoro-4-phenoxyphenyl)-1H-pyrazolo[3,4-d]pyrimidin-1-yl)-3,3-dimethylpiperidine-1-carboxylate (4-j). The Boc-protected amine of 4-j is deprotected in acidic conditions to yield (R)-1-(5,5-dimethylpiperidin-3-yl)-3-(2-fluoro-4-phenoxyphenyl)-1H-pyrazolo[3,4-d]pyrimidin-4-amine (4-k), and the free amine of 4-k is reacted with (E)-2-cyano-4-methyl-4-(4-(oxetan-3-yl)piperazin-1-yl)pent-2-enoic acid (4-f) in DMF in the presence of HATU and a diisopropylethylamine base base to yield (R,E)-2-(5-(4-amino-3-(2-fluoro-4-phenoxyphenyl)-1H-pyrazolo[3,4-d]pyrimidin-1-yl)-3,3-dimethylpiperidine-1-carbonyl)-4-methyl-4-(4-(oxetan-3-yl)piperazin-1-yl)pent-2-enenitrile (4-C).

### Example S16. (R,E)-2-(5-(4-amino-3-(2-fluoro-4-phenoxyphenyl)-1H-pyrazolo[3,4-d]pyrimidin-1-yl)-3,3-difluoropiperidine-1-carbonyl)-4-methyl-4-(4-(oxetan-3-yl)piperazin-1-yl)pent-2-enenitrile (4-D)

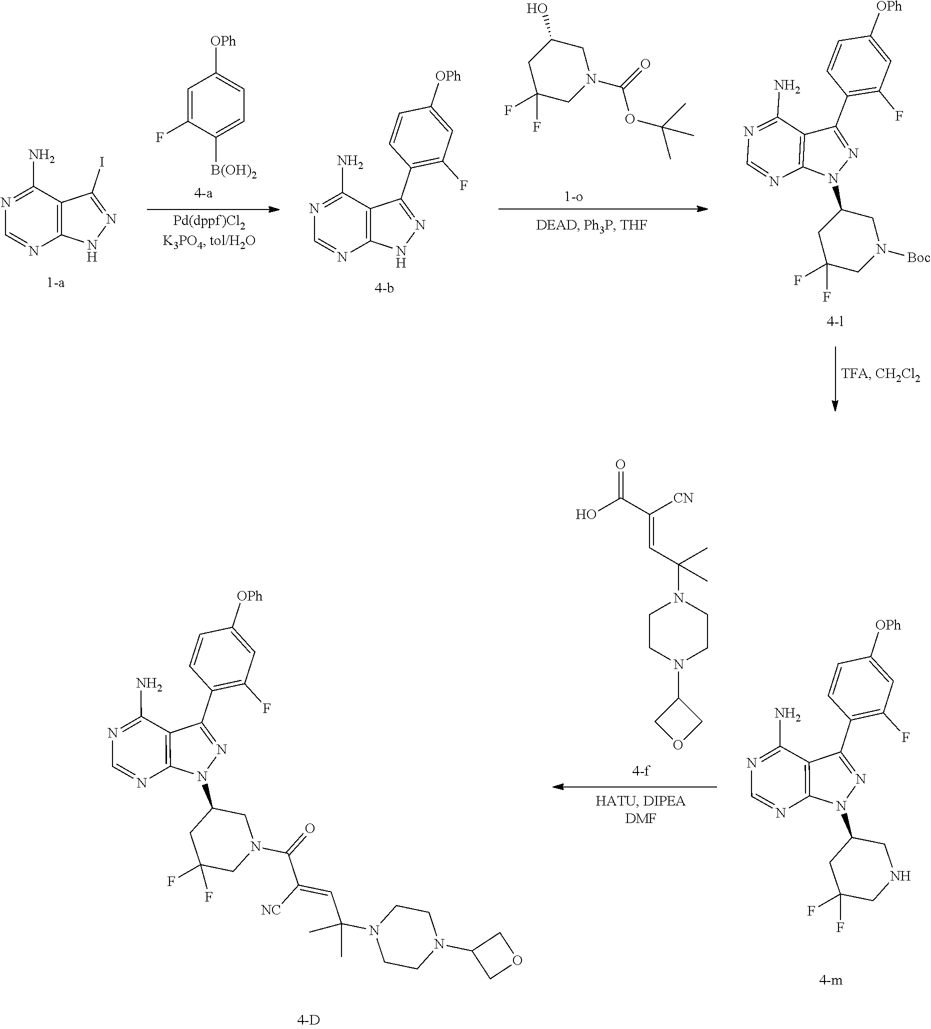

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (S)-3,3-difluoro-5-hydroxypiperidine-1-carboxylate (1-o), diethyl azodicarboxylate, and triphenylphosphine is added. The reaction solution is stirred at room temperature to yield tert-butyl (R)-5-(4-amino-3-(2-fluoro-4-phenoxyphenyl)-1H-pyrazolo[3,4-d]pyrimidin-1-yl)-3,3-difluoropiperidine-1-carboxylate (4-1). The Boc-protected amine of 4-1 is deprotected in acidic conditions to yield (R)-1-(5,5-difluoropiperidin-3-yl)-3-(2-fluoro-4-phenoxyphenyl)-1H-pyrazolo[3,4-d]pyrimidin-4-amine (4-m), and the free amine of 4-m is reacted with (E)-2-cyano-4-methyl-4-(4-(oxetan-3-yl)piperazin-1-yl)pent-2-enoic acid (4-f) in DMF in the presence of HATU and a diisopropylethylamine base base to yield (R,E)-2-(5-(4-amino-3-(2-fluoro-4-phenoxyphenyl)-1H-pyrazolo[3,4-d]pyrimidin-1-yl)-3,3-difluoropiperidine-1-carbonyl)-4-methyl-4-(4-(oxetan-3-yl)piperazin-1-yl)pent-2-enenitrile (4-D).

### Example S17. (E)-2-((3R,5R)-3-(4-amino-3-(2-fluoro-4-phenoxyphenyl)-1H-pyrazolo[3,4-d]pyrimidin-1-yl)-5-hydroxypiperidine-1-carbonyl)-4,4-dimethylpent-2-enenitrile (5-A)

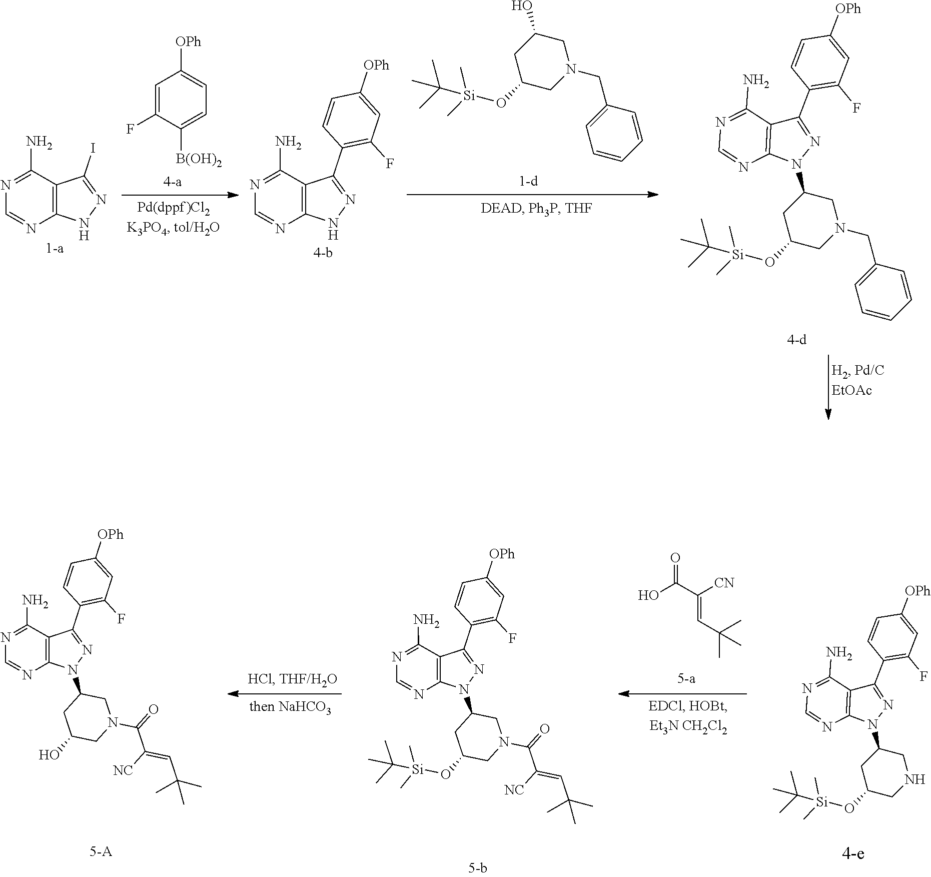

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where (3S,5R)-1-benzyl-5-((tert-butyldimethylsilyl)oxy)piperidin-3-ol (1-d), diethyl azodicarboxylate, and triphenylphosphine is added. The reaction solution is stirred at room temperature to yield 1-((3R,5R)-1-benzyl-5-((tert-butyldimethylsilyl)oxy)piperidin-3-yl)-3-(2-fluoro-4-phenoxyphenyl)-1H-pyrazolo[3,4-d]pyrimidin-4-amine (4-d). 4-d is dissolved in ethyl acetate and hydrogenated with H₂ and Pd/C to yield 1-((3R,5R)-5-((tert-butyldimethylsilyl)oxy)piperidin-3-yl)-3-(2-fluoro-4-phenoxyphenyl)-1H-pyrazolo[3,4-d]pyrimidin-4-amine (4-e). 4-e is coupled with (E)-2-cyano-4,4-dimethylpent-2-enoic acid (5-a) in dichloromethane in the presence of EDCI, HOBt, and a triethylamine base to yield (E)-2-((3R,5R)-3-(4-amino-3-(2-fluoro-4-phenoxyphenyl)-1H-pyrazolo[3,4-d]pyrimidin-1-yl)-5-((tert-butyldimethylsilyl)oxy)piperidine-1-carbonyl)-4,4-dimethylpent-2-enenitrile (5-b). Deprotection of the tert-butyldimethylsilyl-protected alcohol of 5-b in acidic conditions yields (E)-2-((3R,5R)-3-(4-amino-3-(2-fluoro-4-phenoxyphenyl)-1H-pyrazolo[3,4-d]pyrimidin-1-yl)-5-hydroxypiperidine-1-carbonyl)-4,4-dimethylpent-2-enenitrile (5-A).

### Example S18. (E)-2-((3R,5R)-3-(4-amino-3-(2-fluoro-4-phenoxyphenyl)-1H-pyrazolo[3,4-d]pyrimidin-1-yl)-5-methoxypiperidine-1-carbonyl)-4,4-dimethylpent-2-enenitrile (5-B)

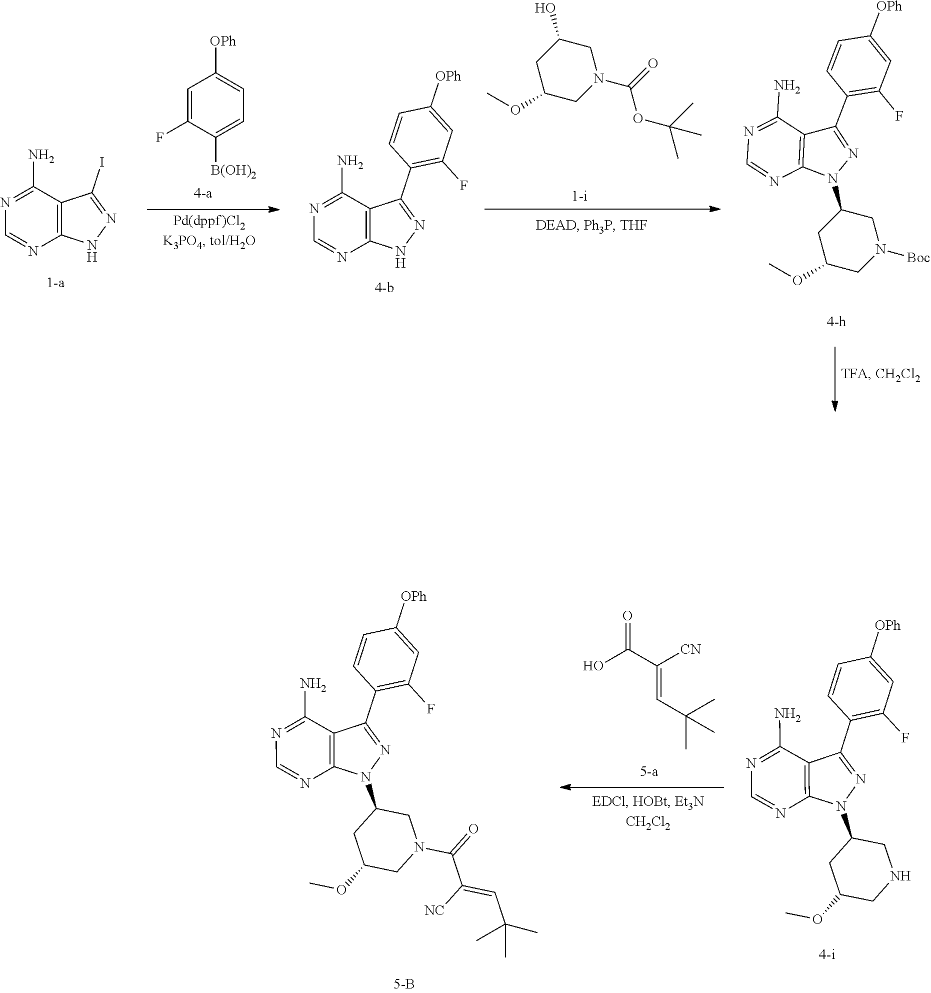

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (3S,5R)-3-hydroxy-5-methoxypiperidine-1-carboxylate (1-i), diethyl azodicarboxylate, and triphenylphosphine is added. The reaction solution is stirred at room temperature to yield tert-butyl (3R,5R)-3-(4-amino-3-(2-fluoro-4-phenoxyphenyl)-1H-pyrazolo[3,4-d]pyrimidin-1-yl)-5-methoxypiperidine-1-carboxylate (4-h). The Boc-protected amine of 4-h is deprotected in acidic conditions to yield 3-(2-fluoro-4-phenoxyphenyl)-1-((3R,5R)-5-methoxypiperidin-3-yl)-1H-pyrazolo[3,4-d]pyrimidin-4-amine (4-i), and the free amine of 4-i is reacted with (E)-2-cyano-4,4-dimethylpent-2-enoic acid (5-a) in dichloromethane in the presence of EDCI, HOBt, and a triethylamine base to yield (E)-2-((3R,5R)-3-(4-amino-3-(2-fluoro-4-phenoxyphenyl)-1H-pyrazolo[3,4-d]pyrimidin-1-yl)-5-methoxypiperidine-1-carbonyl)-4,4-dimethylpent-2-enenitrile (5-B).

### Example S19. (E)-2-((3R,5R)-3-(4-amino-3-(2-fluoro-4-phenoxyphenyl)-1H-pyrazolo[3,4-d]pyrimidin-1-yl)-5-methoxypiperidine-1-carbonyl)-4,4-dimethylpent-2-enenitrile (5-C)

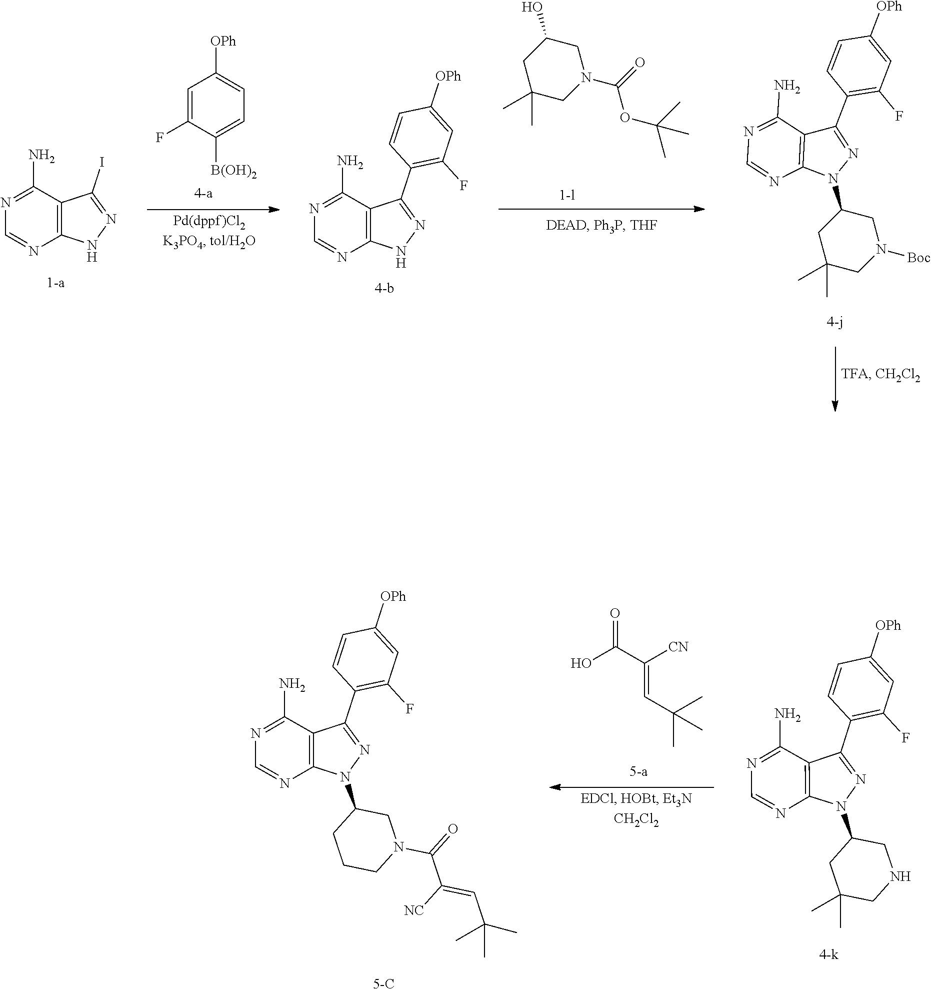

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (S)-5-hydroxy-3,3-dimethylpiperidine-1-carboxylate (1-1), diethyl azodicarboxylate, and triphenylphosphine is added. The reaction solution is stirred at room temperature to yield tert-butyl (R)-5-(4-amino-3-(2-fluoro-4-phenoxyphenyl)-1H-pyrazolo[3,4-d]pyrimidin-1-yl)-3,3-dimethylpiperidine-1-carboxylate (4-j). The Boc-protected amine of 4-j is deprotected in acidic conditions to yield (R)-1-(5,5-dimethylpiperidin-3-yl)-3-(2-fluoro-4-phenoxyphenyl)-1H-pyrazolo[3,4-d]pyrimidin-4-amine (4-k), and the free amine of 4-k is reacted with (E)-2-cyano-4,4-dimethylpent-2-enoic acid (5-a) in dichloromethane in the presence of EDCI, HOBt, and a triethylamine base to yield (R,E)-2-(5-(4-amino-3-(2-fluoro-4-phenoxyphenyl)-1H-pyrazolo[3,4-d]pyrimidin-1-yl)-3,3-dimethylpiperidine-1-carbonyl)-4,4-dimethylpent-2-enenitrile (5-C).

### Example S20. (R,E)-2-(5-(4-amino-3-(2-fluoro-4-phenoxyphenyl)-1H-pyrazolo[3,4-d]pyrimidin-1-yl)-3,3-difluoropiperidine-1-carbonyl)-4,4-dimethylpent-2-enenitrile (5-D)

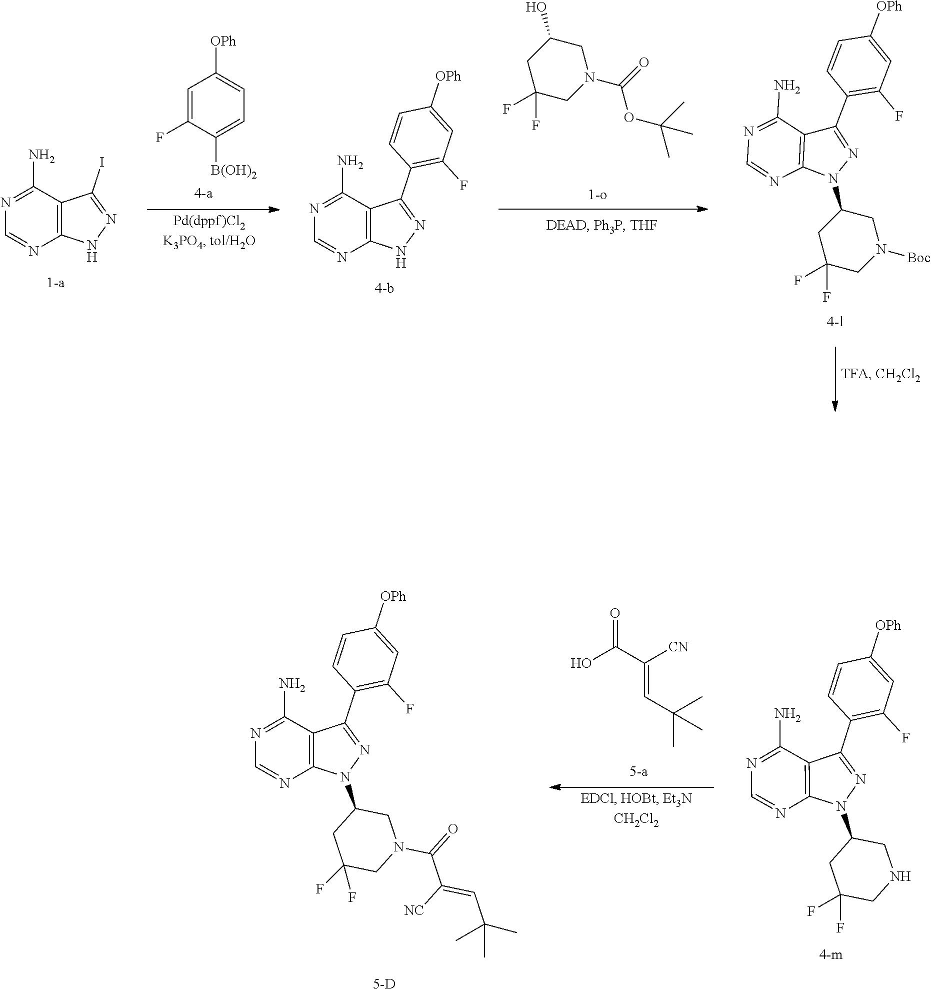

To a solution of 3-iodo-1H-pyrazolo[3,4-d]pyrimidin-4-amine (1-a) in toluene and water is added (2-fluoro-4-phenoxyphenyl)boronic acid (4-a), Pd(dppf)Cl₂, and K₃PO₄. Stirring the reaction solution at 90-100° C. yields 3-(2-fluoro-4-phenoxyphenyl)-1H-pyrazolo[3,4-d]pyrimidin-4-amine (4-b). 4-b is dissolved in THE and is cooled to 0° C., where tert-butyl (S)-3,3-difluoro-5-hydroxypiperidine-1-carboxylate (1-o), diethyl azodicarboxylate, and triphenylphosphine is added. The reaction solution is stirred at room temperature to yield tert-butyl (R)-5-(4-amino-3-(2-fluoro-4-phenoxyphenyl)-1H-pyrazolo[3,4-d]pyrimidin-1-yl)-3,3-difluoropiperidine-1-carboxylate (4-1). The Boc-protected amine of 4-1 is deprotected in acidic conditions to yield (R)-1-(5,5-difluoropiperidin-3-yl)-3-(2-fluoro-4-phenoxyphenyl)-1H-pyrazolo[3,4-d]pyrimidin-4-amine (4-m), and the free amine of 4-m is reacted with (E)-2-cyano-4,4-dimethylpent-2-enoic acid (5-a) in dichloromethane in the presence of EDCI, HOBt, and a triethylamine base to yield (R,E)-2-(5-(4-amino-3-(2-fluoro-4-phenoxyphenyl)-1H-pyrazolo[3,4-d]pyrimidin-1-yl)-3,3-difluoropiperidine-1-carbonyl)-4,4-dimethylpent-2-enenitrile (5-D).

### Example S21. 1-(4-(((6-amino-5-(4-phenoxyphenyl)pyrimidin-4-yl)amino)methyl)-3-hydroxypiperidin-1-yl)prop-2-en-1-one (6-A)

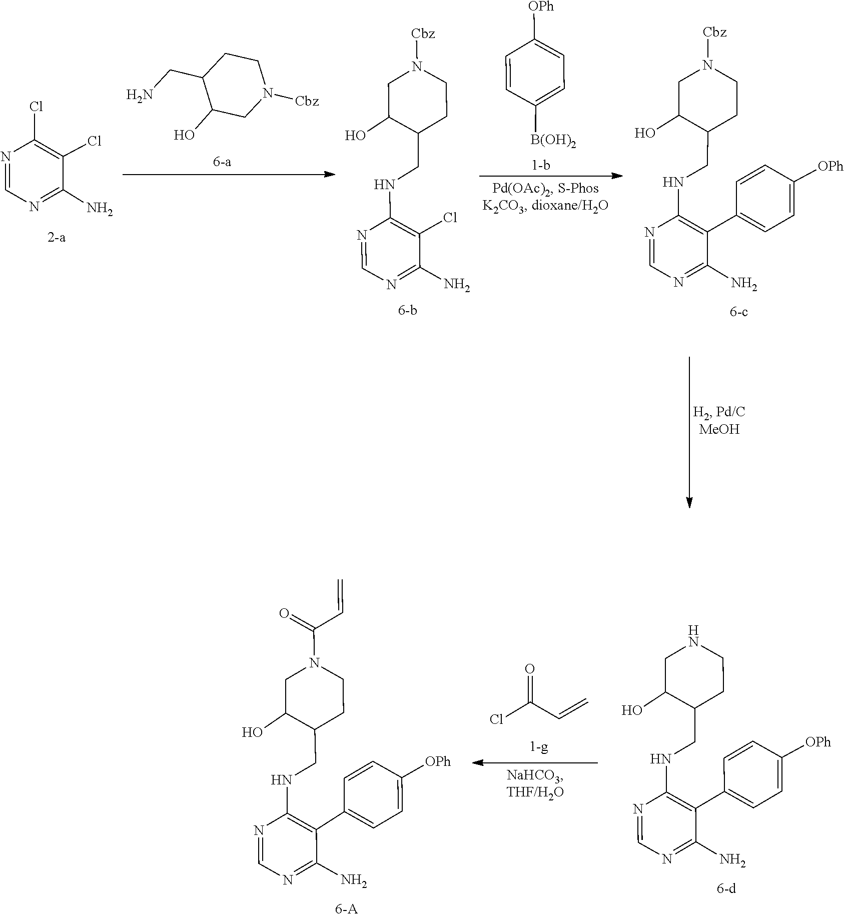

2,4-Dichloro-3-nitropyridine (2-a) and benzyl 4-(aminomethyl)-3-hydroxypiperidine-1-carboxylate (6-a) are coupled in the presence of DBU in DMF to yield benzyl 4-(((6-amino-5-chloropyrimidin-4-yl)amino)methyl)-3-hydroxypiperidine-1-carboxylate (6-b), which subsequently undergoes a cross-coupling reaction with (4-phenoxyphenyl)boronic acid (1-b) with Cu(OAc)₂, TEMPO, and triethylamine in dichloromethane to yield benzyl 4-(((6-amino-5-(4-phenoxyphenyl)pyrimidin-4-yl)amino)methyl)-3-hydroxypiperidine-1-carboxylate (6-c). 6-c is dissolved in methanol and hydrogenated with H₂ and Pd/C to yield 4-(((6-amino-5-(4-phenoxyphenyl)pyrimidin-4-yl)amino)methyl)piperidin-3-ol (6-d). 6-d is coupled with acryloyl chloride (1-g) in the presence of sodium bicarbonate in THF/H₂O to yield 1-(4-(((6-amino-5-(4-phenoxyphenyl)pyrimidin-4-yl)amino)methyl)-3-hydroxypiperidin-1-yl)prop-2-en-1-one (6-A).

### Example S22. 1-(4-(((6-amino-5-(4-phenoxyphenyl)pyrimidin-4-yl)amino)methyl)-3-methoxypiperidin-1-yl)prop-2-en-1-one (6-B)

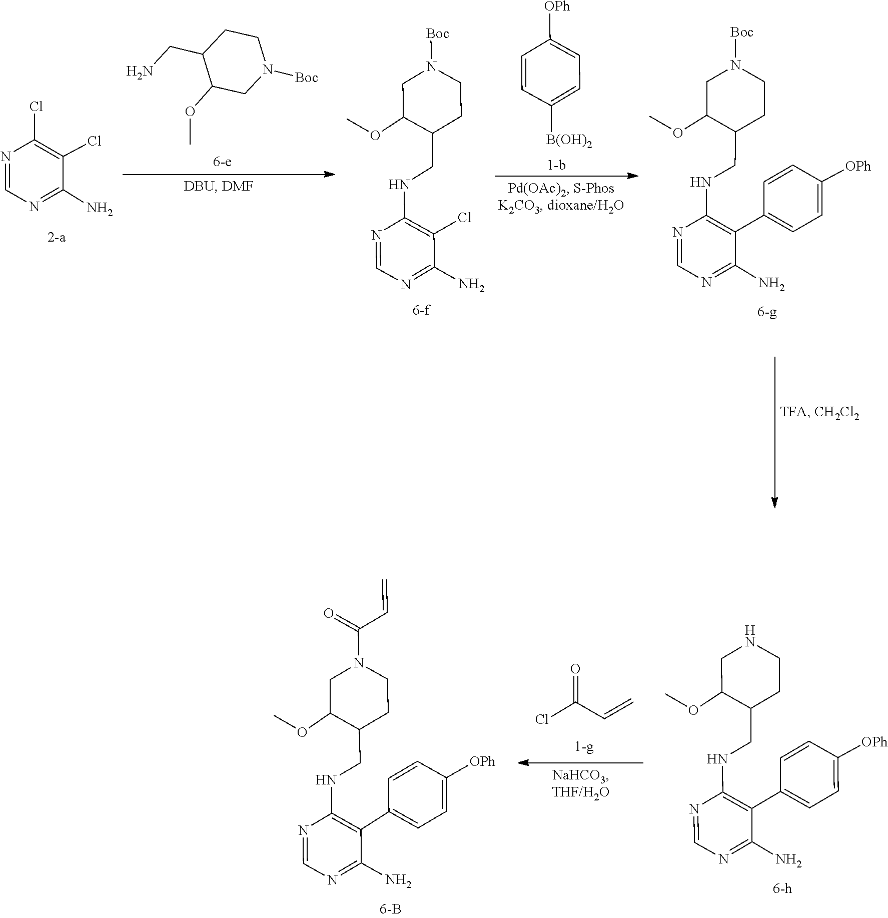

2,4-Dichloro-3-nitropyridine (2-a) and tert-butyl 4-(aminomethyl)-3-methoxypiperidine-1-carboxylate (6-e) are coupled in the presence of DBU in DMF to yield tert-butyl 4-(((6-amino-5-chloropyrimidin-4-yl)amino)methyl)-3-methoxypiperidine-1-carboxylate (6-f), which subsequently undergoes a cross-coupling reaction with (4-phenoxyphenyl)boronic acid (1-b) with Cu(OAc)₂, TEMPO, and triethylamine in dichloromethane to yield tert-butyl 4-(((6-amino-5-(4-phenoxyphenyl)pyrimidin-4-yl)amino)methyl)-3-methoxypiperidine-1-carboxylate (6-g). The Boc-protected amine of 6-g is deprotected in acidic conditions to yield N4-((3-methoxypiperidin-4-yl)methyl)-5-(4-phenoxyphenyl)pyrimidine-4,6-diamine (6-h), and the free amine of 6-h is coupled with acryloyl chloride (1-g) in the presence of sodium bicarbonate in THF/H₂O to yield 1-(4-(((6-amino-5-(4-phenoxyphenyl)pyrimidin-4-yl)amino)methyl)-3-methoxypiperidin-1-yl)prop-2-en-1-one (6-B).

### Example S23. 1-(4-(((6-amino-5-(4-phenoxyphenyl)pyrimidin-4-yl)amino)methyl)-3,3-dimethylpiperidin-1-yl)prop-2-en-1-one (6-C)

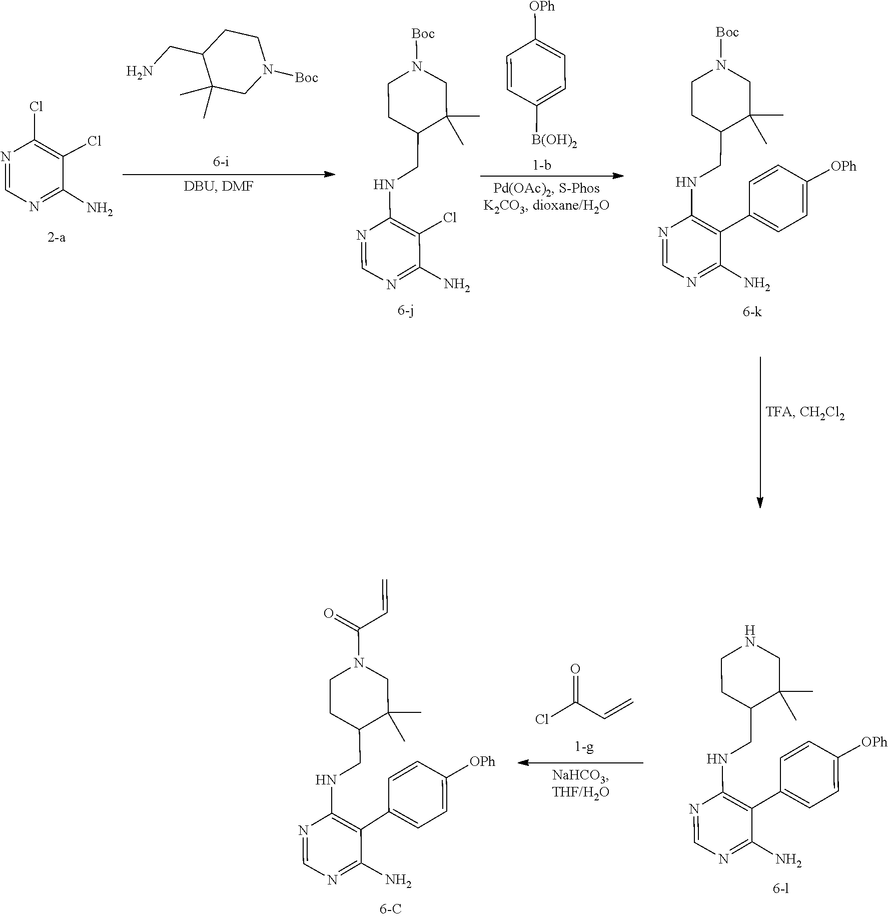

2,4-Dichloro-3-nitropyridine (2-a) and tert-butyl 4-(aminomethyl)-3,3-dimethylpiperidine-1-carboxylate (6-i) are coupled in the presence of DBU in DMF to yield tert-butyl 4-(((6-amino-5-chloropyrimidin-4-yl)amino)methyl)-3,3-dimethylpiperidine-1-carboxylate (6-i), which subsequently undergoes a cross-coupling reaction with (4-phenoxyphenyl)boronic acid (1-b) with Cu(OAc)₂, TEMPO, and triethylamine in dichloromethane to yield tert-butyl 4-(((6-amino-5-(4-phenoxyphenyl)pyrimidin-4-yl)amino)methyl)-3,3-dimethylpiperidine-1-carboxylate (6-k). The Boc-protected amine of 6-k is deprotected in acidic conditions to yield N4-((3,3-dimethylpiperidin-4-yl)methyl)-5-(4-phenoxyphenyl)pyrimidine-4,6-diamine (6-1), and the free amine of 6-1 is coupled with acryloyl chloride (1-g) in the presence of sodium bicarbonate in THF/H₂O to yield 1-(4-(((6-amino-5-(4-phenoxyphenyl)pyrimidin-4-yl)amino)methyl)-3,3-dimethylpiperidin-1-yl)prop-2-en-1-one (6-C).

### Example S24. 1-(4-(((6-amino-5-(4-phenoxyphenyl)pyrimidin-4-yl)amino)methyl)-3,3-dimethylpiperidin-1-yl)prop-2-en-1-one (6-D)

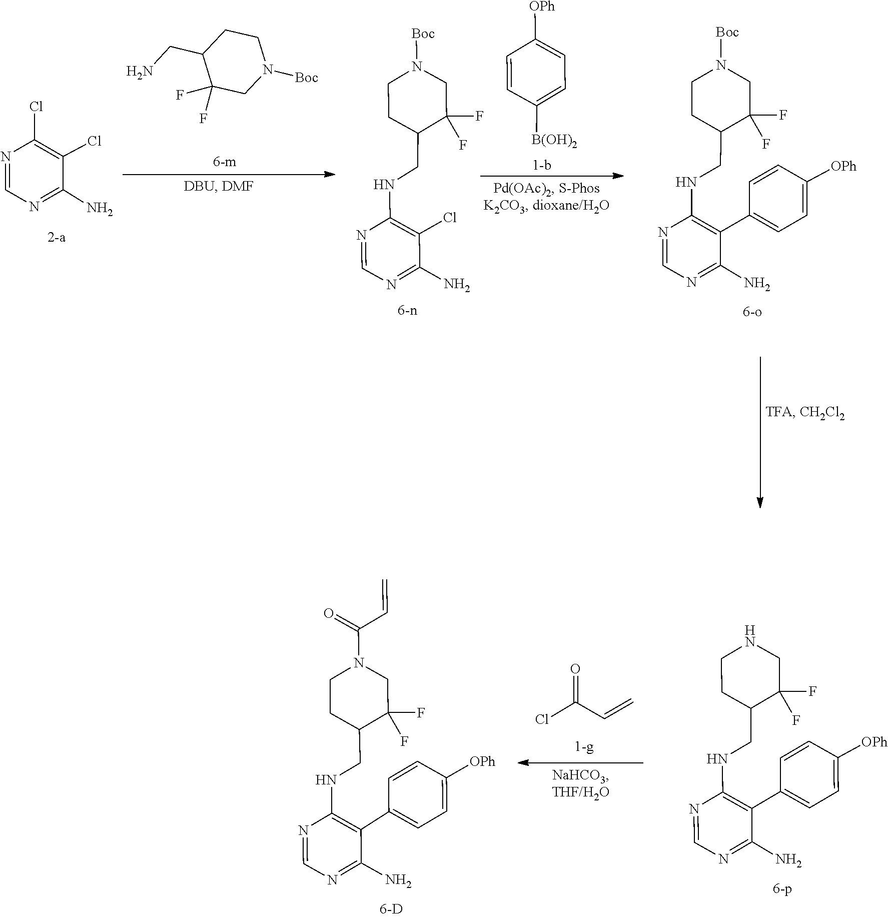

2,4-Dichloro-3-nitropyridine (2-a) and tert-butyl 4-(aminomethyl)-3,3-difluoropiperidine-1-carboxylate (6-m) are coupled in the presence of DBU in DMF to yield tert-butyl 4-(((6-amino-5-chloropyrimidin-4-yl)amino)methyl)-3,3-difluoropiperidine-1-carboxylate (6-n), which subsequently undergoes a cross-coupling reaction with (4-phenoxyphenyl)boronic acid (1-b) with Cu(OAc)₂, TEMPO, and triethylamine in dichloromethane to yield tert-butyl 4-(((6-amino-5-(4-phenoxyphenyl)pyrimidin-4-yl)amino)methyl)-3,3-difluoropiperidine-1-carboxylate (6-0). The Boc-protected amine of 6-o is deprotected in acidic conditions to yield N4-((3,3-difluoropiperidin-4-yl)methyl)-5-(4-phenoxyphenyl)pyrimidine-4,6-diamine (6-p), and the free amine of 6-p is coupled with acryloyl chloride (1-g) in the presence of sodium bicarbonate in THF/H₂O to yield 1-(4-(((6-amino-5-(4-phenoxyphenyl)pyrimidin-4-yl)amino)methyl)-3,3-difluoropiperidin-1-yl)prop-2-en-1-one (6-D).

### Example S25. 4-((3S,5S)-3-(but-2-ynamido)-5-hydroxypiperidin-1-yl)-5-fluoro-2,3-dimethyl-1H-indole-7-carboxamide (7-A)

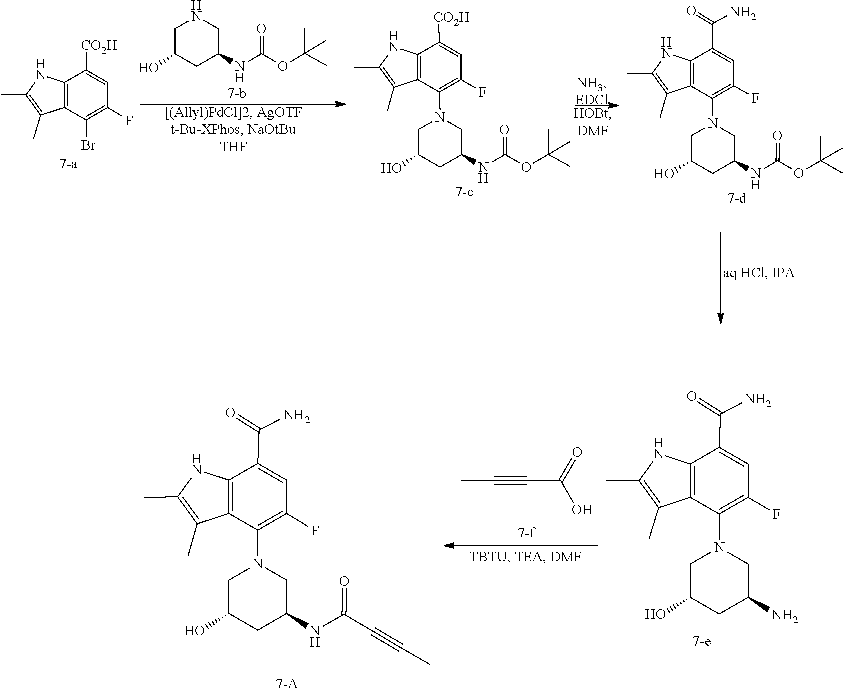

The coupling between 4-bromo-5-fluoro-2,3-dimethyl-1H-indole-7-carboxylic acid (7-a) and tert-butyl ((3S,5S)-5-hydroxypiperidin-3-yl)carbamate (7-b) in THE with allylpalladium (II) chloride dimer, silver trifluoromethanesulfonate, t-Bu-XPhos, and a sodium tert-butoxide base yields 4-((3S,5S)-3-((tert-butoxycarbonyl)amino)-5-hydroxypiperidin-1-yl)-5-fluoro-2,3-dimethyl-1H-indole-7-carboxylic acid (7-c). The carboxylic acid of 7-c is converted to an amide by reacting with NH₃ in the presence of EDCI and HOBt in DMF to yield tert-butyl ((3S,5S)-1-(7-carbamoyl-5-fluoro-2,3-dimethyl-1H-indol-4-yl)-5-hydroxypiperidin-3-yl)carbamate (7-d). The Boc-protected amine of 7-d is deprotected in acidic conditions to yield 4-((3S,5S)-3-amino-5-hydroxypiperidin-1-yl)-5-fluoro-2,3-dimethyl-1H-indole-7-carboxamide (7-e), and the free amine of 7-e is coupled with but-2-ynoic acid (7-f) in the presence of TBTU and a triethylamine base in DMF to yield 4-((3S,5S)-3-(but-2-ynamido)-5-hydroxypiperidin-1-yl)-5-fluoro-2,3-dimethyl-1H-indole-7-carboxamide (7-A).

### Example S26. 4-((3S,5S)-3-(but-2-ynamido)-5-methoxypiperidin-1-yl)-5-fluoro-2,3-dimethyl-1H-indole-7-carboxamide (7-B)

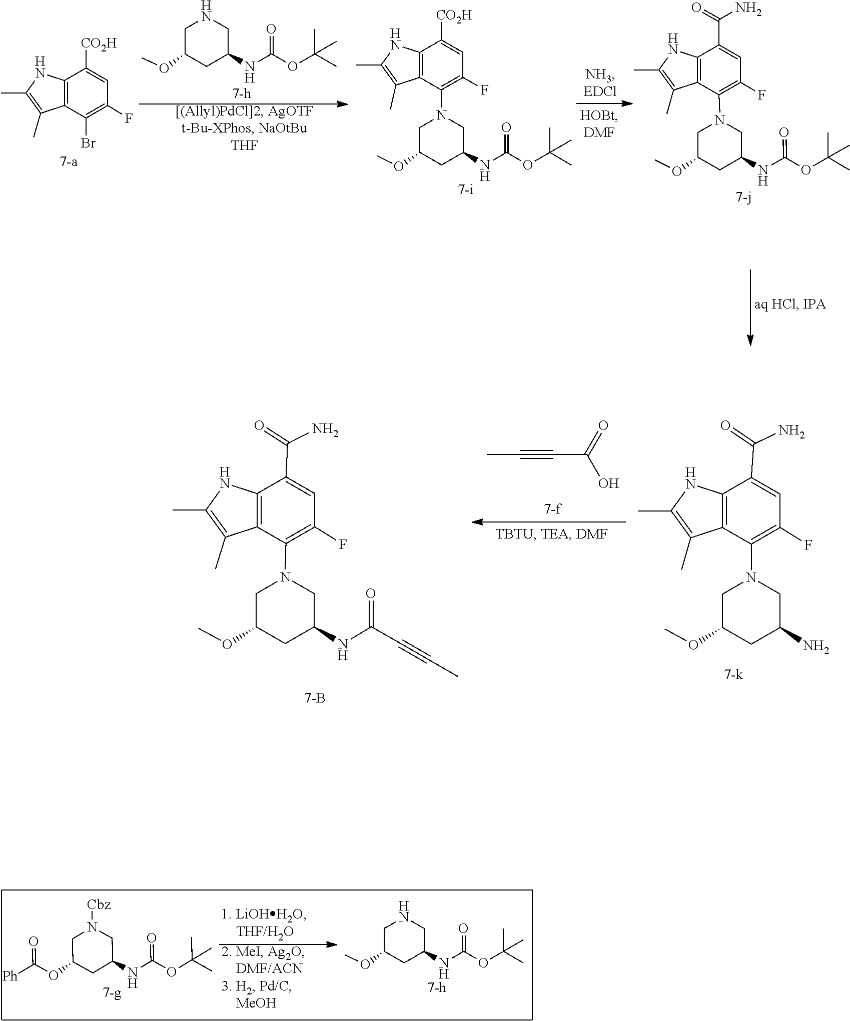

By reacting benzyl (3S,5S)-3-(benzoyloxy)-5-((tert-butoxycarbonyl)amino)piperidine-1-carboxylate (7-g) with LiOH·H₂O in THF/H₂O and Mel and Ag₂O in DMF/ACN, the phenyl ester of 7-g is converted to a methoxy. Subsequent deprotection of the benzylcarbonyl-protected amine with H₂ and Pd/C in MeOH yields tert-butyl ((3S,5S)-5-methoxypiperidin-3-yl)carbamate (7-h). The coupling between 4-bromo-5-fluoro-2,3-dimethyl-1H-indole-7-carboxylic acid (7-a) and 7-h in THE with allylpalladium (II) chloride dimer, silver trifluoromethanesulfonate, t-Bu-XPhos, and a sodium tert-butoxide base yields 4-((3S,5S)-3-((tert-butoxycarbonyl)amino)-5-methoxypiperidin-1-yl)-5-fluoro-2,3-dimethyl-1H-indole-7-carboxylic acid (7-i). The carboxylic acid of 7-i is converted to an amide by reacting with NH₃ in the presence of EDCI and HOBt in DMF to yield tert-butyl ((3S,5S)-1-(7-carbamoyl-5-fluoro-2,3-dimethyl-1H-indol-4-yl)-5-methoxypiperidin-3-yl)carbamate (7-j). The Boc-protected amine of 7-j is deprotected in acidic conditions to yield 4-((3S,5S)-3-amino-5-methoxypiperidin-1-yl)-5-fluoro-2,3-dimethyl-1H-indole-7-carboxamide (7-k), and the free amine of 7-k is coupled with but-2-ynoic acid (7-f) in the presence of TBTU and a triethylamine base in DMF to yield 4-((3S,5S)-3-(but-2-ynamido)-5-methoxypiperidin-1-yl)-5-fluoro-2,3-dimethyl-1H-indole-7-carboxamide (7-B).

### Example S27. (S)-4-(5-(but-2-ynamido)-3,3-dimethylpiperidin-1-yl)-5-fluoro-2,3-dimethyl-1H-indole-7-carboxamide (7-C)

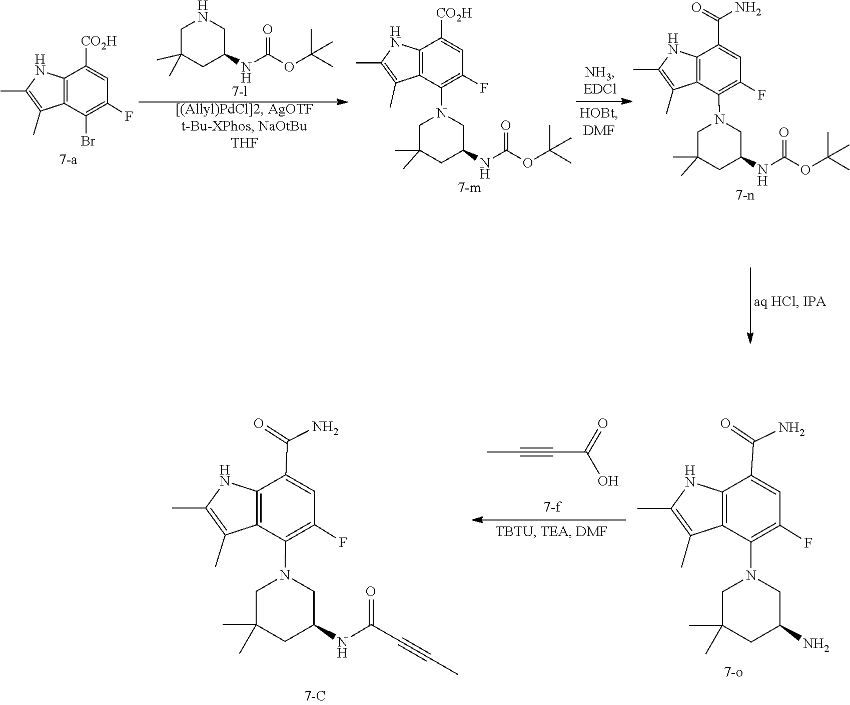

The coupling between 4-bromo-5-fluoro-2,3-dimethyl-1H-indole-7-carboxylic acid (7-a) and tert-butyl (S)-(5,5-dimethylpiperidin-3-yl)carbamate (7-1) in THF with allylpalladium (II) chloride dimer, silver trifluoromethanesulfonate, t-Bu-XPhos, and a sodium tert-butoxide base yields (S)-4-(5-((tert-butoxycarbonyl)amino)-3,3-dimethylpiperidin-1-yl)-5-fluoro-2,3-dimethyl-1H-indole-7-carboxylic acid (7-m). The carboxylic acid of 7-m is converted to an amide by reacting with NH₃ in the presence of EDCI and HOBt in DMF to yield tert-butyl (S)-(1-(7-carbamoyl-5-fluoro-2,3-dimethyl-1H-indol-4-yl)-5,5-dimethylpiperidin-3-yl)carbamate (7-n). The Boc-protected amine of 7-n is deprotected in acidic conditions to yield (S)-4-(5-amino-3,3-dimethylpiperidin-1-yl)-5-fluoro-2,3-dimethyl-1H-indole-7-carboxamide (7-o), and the free amine of 7-o is coupled with but-2-ynoic acid (7-f) in the presence of TBTU and a triethylamine base in DMF to yield (S)-4-(5-(but-2-ynamido)-3,3-dimethylpiperidin-1-yl)-5-fluoro-2,3-dimethyl-1H-indole-7-carboxamide (7-C).

### Example S28. (S)-4-(5-(but-2-ynamido)-3,3-difluoropiperidin-1-yl)-5-fluoro-2,3-dimethyl-1H-indole-7-carboxamide (7-D)

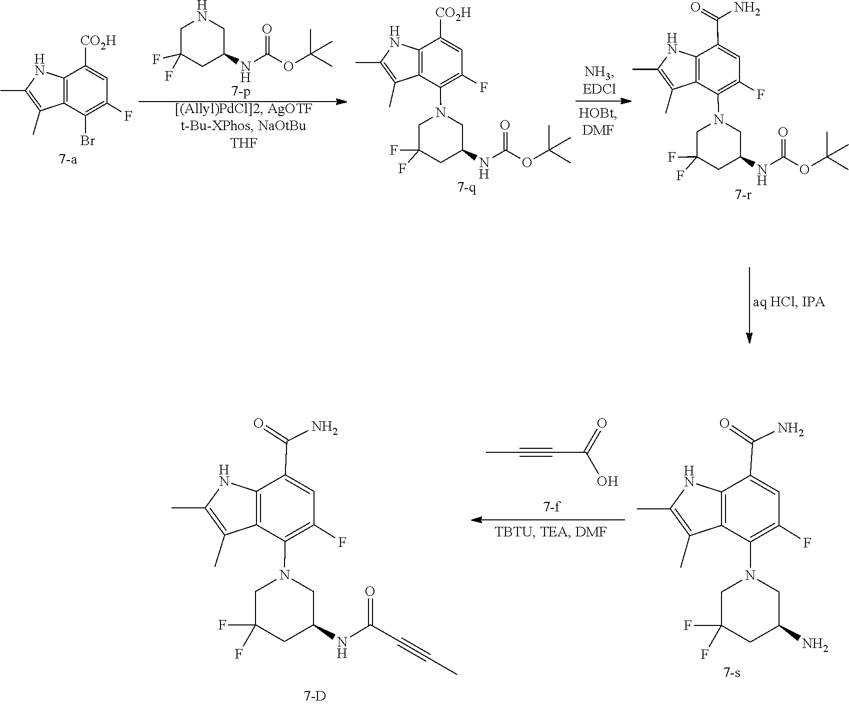

The coupling between 4-bromo-5-fluoro-2,3-dimethyl-1H-indole-7-carboxylic acid (7-a) and tert-butyl (S)-(5,5-difluoropiperidin-3-yl)carbamate (7-p) in THE with allylpalladium (II) chloride dimer, silver trifluoromethanesulfonate, t-Bu-XPhos, and a sodium tert-butoxide base yields (S)-4-(5-((tert-butoxycarbonyl)amino)-3,3-difluoropiperidin-1-yl)-5-fluoro-2,3-dimethyl-1H-indole-7-carboxylic acid (7-q). The carboxylic acid of 7-q is converted to an amide by reacting with NH₃ in the presence of EDCI and HOBt in DMF to yield tert-butyl (S)-(1-(7-carbamoyl-5-fluoro-2,3-dimethyl-1H-indol-4-yl)-5,5-difluoropiperidin-3-yl)carbamate (7-r). The Boc-protected amine of 7-r is deprotected in acidic conditions to yield (S)-4-(5-amino-3,3-difluoropiperidin-1-yl)-5-fluoro-2,3-dimethyl-1H-indole-7-carboxamide (7-s), and the free amine of 7-s is coupled with but-2-ynoic acid (7-f) in the presence of TBTU and a triethylamine base in DMF to yield (S)-4-(5-(but-2-ynamido)-3,3-difluoropiperidin-1-yl)-5-fluoro-2,3-dimethyl-1H-indole-7-carboxamide (7-D).

### Example S29. 6-(1-acryloyl-3-hydroxypiperidin-4-yl)-2-(4-phenoxyphenyl)nicotinamide (8-A)

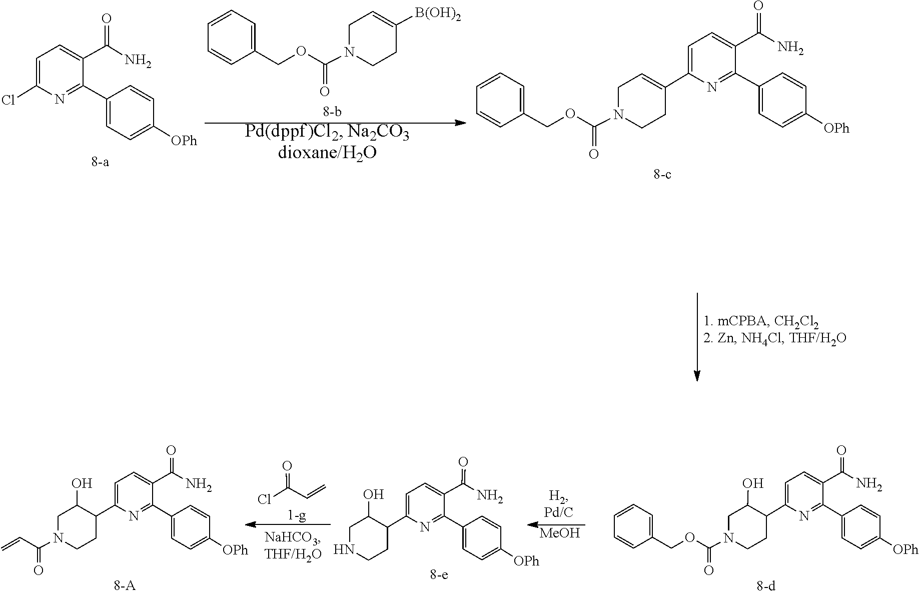

To a solution of 6-chloro-2-(4-phenoxyphenyl)nicotinamide (8-a) in dioxane and water is added (1-((benzyloxy)carbonyl)-1,2,3,6-tetrahydropyridin-4-yl)boronic acid (8-b), Pd(dppf)Cl₂, and Na₂CO₃. Stirring the reaction solution at 90-100° C. yields benzyl 5-carbamoyl-6-(4-phenoxyphenyl)-3′,6′-dihydro-[2,4′-bipyridine]-1′(2′H)-carboxylate (8-c). Subsequent reaction of 8-c with mCPBA in dichloromethane followed by Zn and NH₄Cl in THF/H₂O yields an alcohol-substituted benzyl 4-(5-carbamoyl-6-(4-phenoxyphenyl)pyridin-2-yl)-3-hydroxypiperidine-1-carboxylate (8-d). Deprotection of the amine of 8-d with H₂ and Pd/C in MeOH yields 6-(3-hydroxypiperidin-4-yl)-2-(4-phenoxyphenyl)nicotinamide (8-e), which is subsequently reacted with acryloyl chloride (1-g) in the presence of a sodium bicarbonate base in THF/H₂O to yield 6-(1-acryloyl-3-hydroxypiperidin-4-yl)-2-(4-phenoxyphenyl)nicotinamide (8-A).

### Example S30. 6-(1-acryloyl-3-methoxypiperidin-4-yl)-2-(4-phenoxyphenyl)nicotinamide (8-B)

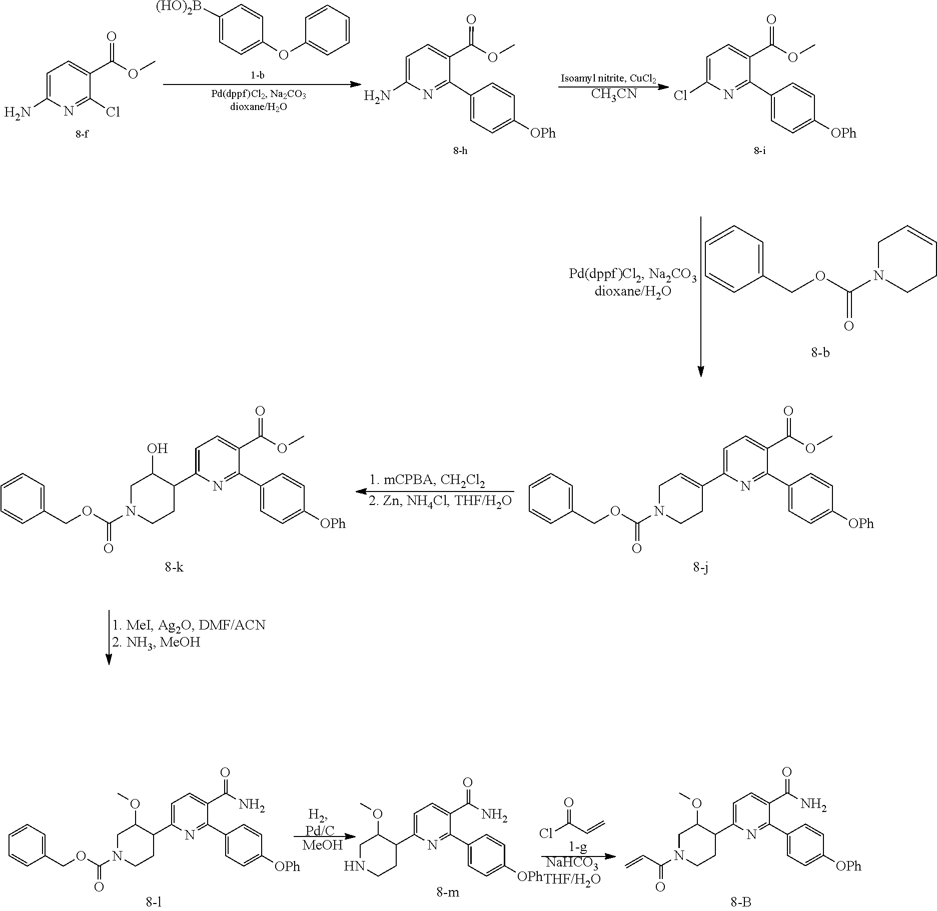

To a solution of methyl 6-amino-2-chloronicotinate (8-f) in dioxane and water is added (4-phenoxyphenyl)boronic acid (1-b), Pd(dppf)Cl₂, and Na₂CO₃. Stirring the reaction solution at 90-100° C. yields methyl 6-amino-2-(4-phenoxyphenyl)nicotinate (8-h). The amine of 8-h is converted to a chloro by reacting it with isoamyl nitrite and CuCl₂ in acetonitrile which yields methyl 6-chloro-2-(4-phenoxyphenyl)nicotinate (8-i). Coupling of 8-i with (1-((benzyloxy)carbonyl)-1,2,3,6-tetrahydropyridin-4-yl)boronic acid (8-b) by reacting in dioxane and water with Pd(dppf)Cl₂ and Na₂CO₃ yields 1′-benzyl 5-methyl 6-(4-phenoxyphenyl)-3′,6′-dihydro-[2,4′-bipyridine]-1′,5(2′H)-dicarboxylate (8-j). Subsequent reaction of 8-j with mCPBA in dichloromethane followed by Zn and NH₄Cl in THF/H₂O yields an alcohol-substituted methyl 6-(1-((benzyloxy)carbonyl)-3-hydroxypiperidin-4-yl)-2-(4-phenoxyphenyl)nicotinate (8-k). Subsequent reaction of 8-k with Mel and Ag₂O in DMF/ACN and NH₃ in MeOH yields benzyl 4-(5-carbamoyl-6-(4-phenoxyphenyl)pyridin-2-yl)-3-methoxypiperidine-1-carboxylate (8-1). Deprotection of the amine of 8-1 with H₂ and Pd/C in MeOH yields 6-(3-methoxypiperidin-4-yl)-2-(4-phenoxyphenyl)nicotinamide (8-m), which is subsequently reacted with acryloyl chloride (1-g) in the presence of a sodium bicarbonate base in THF/H₂O to yield 6-(1-acryloyl-3-methoxypiperidin-4-yl)-2-(4-phenoxyphenyl)nicotinamide (8-B).

### Example S31. 6-(1-acryloyl-3,3-dimethylpiperidin-4-yl)-2-(4-phenoxyphenyl)nicotinamide

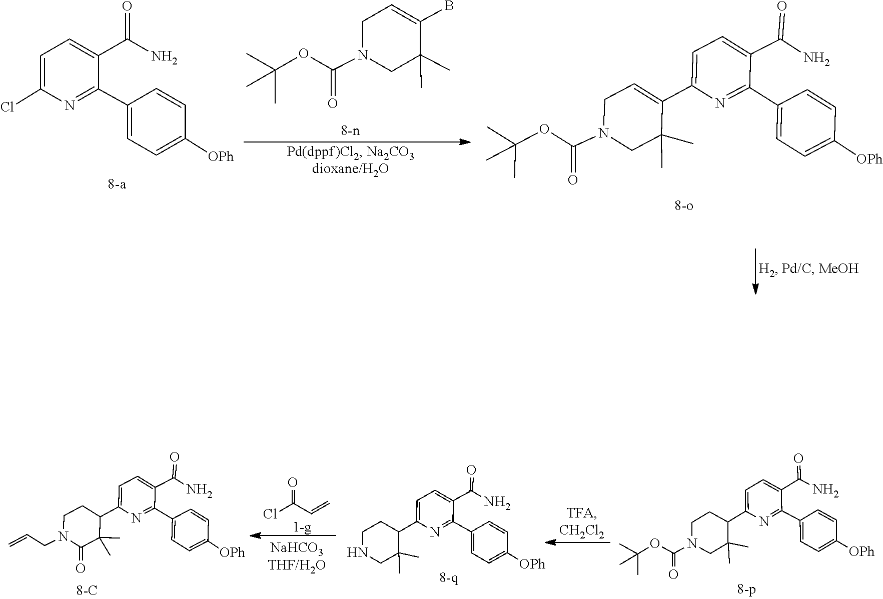

To a solution of 6-chloro-2-(4-phenoxyphenyl)nicotinamide (8-a) in dioxane and water is added (1-(tert-butoxycarbonyl)-3,3-dimethyl-1,2,3,6-tetrahydropyridin-4-yl)boronic acid (8-n), Pd(dppf)Cl₂, and Na₂CO₃. Stirring the reaction solution at 90-100° C. yields tert-butyl 5-carbamoyl-3′,3′-dimethyl-6-(4-phenoxyphenyl)-3′,6′-dihydro-[2,4′-bipyridine]-1′(2′H)-carboxylate (8-o). 8-o undergoes a reaction with H₂ and Pd/C in MeOH to yield tert-butyl 4-(5-carbamoyl-6-(4-phenoxyphenyl)pyridin-2-yl)-3,3-dimethylpiperidine-1-carboxylate (8-p). 8-p is deprotected with TFA in dichloromethane to yield 6-(3,3-dimethylpiperidin-4-yl)-2-(4-phenoxyphenyl)nicotinamide (8-q). 8-q is subsequently reacted with acryloyl chloride (1-g) in the presence of a sodium bicarbonate base in THF/H₂O to yield 6-(1-acryloyl-3,3-dimethylpiperidin-4-yl)-2-(4-phenoxyphenyl)nicotinamide (8-C).

### Example S32. 6-(1-acryloyl-3,3-difluoropiperidin-4-yl)-2-(4-phenoxyphenyl)nicotinamide (8-D)

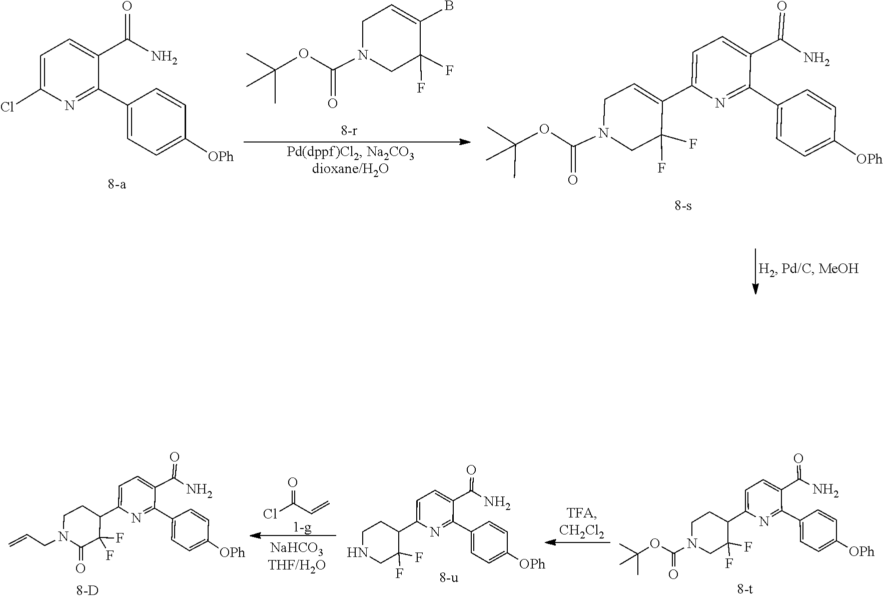

To a solution of 6-chloro-2-(4-phenoxyphenyl)nicotinamide (8-a) in dioxane and water is added (1-(tert-butoxycarbonyl)-3,3-difluoro-1,2,3,6-tetrahydropyridin-4-yl)boronic acid (8-r), Pd(dppf)Cl₂, and Na₂CO₃. Stirring the reaction solution at 90-100° C. yields tert-butyl 5-carbamoyl-3′,3′-difluoro-6-(4-phenoxyphenyl)-3′,6′-dihydro-[2,4′-bipyridine]-1′(2′H)-carboxylate (8-s). 8-s is reacted with H₂ and Pd/C in MeOH to yield tert-butyl 4-(5-carbamoyl-6-(4-phenoxyphenyl)pyridin-2-yl)-3,3-difluoropiperidine-1-carboxylate (8-t). 8-t is deprotected with TFA in dichloromethane to yield 6-(3,3-difluoropiperidin-4-yl)-2-(4-phenoxyphenyl)nicotinamide (8-u). 8-u is subsequently reacted with acryloyl chloride (1-g) in the presence of a sodium bicarbonate base in THF/H₂O to yield 6-(1-acryloyl-3,3-difluoropiperidin-4-yl)-2-(4-phenoxyphenyl)nicotinamide (8-D).

### Example S33. (S)-7-((3R,4R)-1-acryloyl-3-hydroxypiperidin-4-yl)-2-(4-phenoxyphenyl)-4,5,6,7-tetrahydropyrazolo[1,5-a]pyrimidine-3-carboxamide & (S)-7-((3R,4S)-1-acryloyl-3-hydroxypiperidin-4-yl)-2-(4-phenoxyphenyl)-4,5,6,7-tetrahydropyrazolo[1,5-a]pyrimidine-3-carboxamide (9-A & 9-A′)

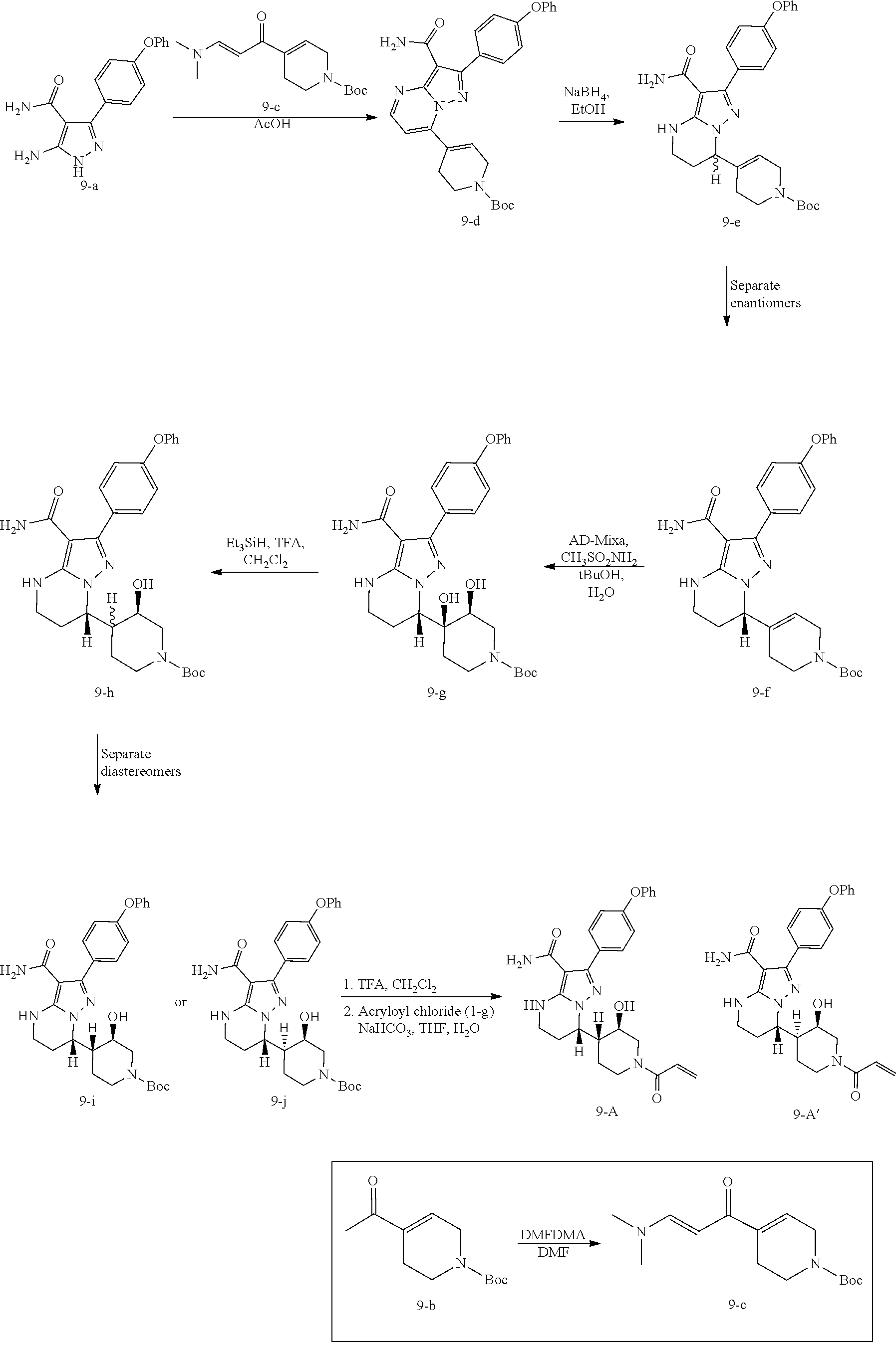

Reacting tert-butyl 4-acetyl-3,6-dihydropyridine-1(2H)-carboxylate (9-b) with N,N-dimethylformamide dimethyl acetal in DMF yields tert-butyl (E)-4-(3-(dimethylamino)acryloyl)-3,6-dihydropyridine-1(2H)-carboxylate (9-c). The coupling between 5-amino-3-(4-phenoxyphenyl)-1H-pyrazole-4-carboxamide (9-a) and 9-c in acetic acid yields tert-butyl 4-(3-carbamoyl-2-(4-phenoxyphenyl)pyrazolo[1,5-a]pyrimidin-7-yl)-3,6-dihydropyridine-1(2H)-carboxylate (9-d), which is reduced with sodium borohydride in ethanol to yield tert-butyl 4-(3-carbamoyl-2-(4-phenoxyphenyl)-4,5,6,7-tetrahydropyrazolo[1,5-a]pyrimidin-7-yl)-3,6-dihydropyridine-1(2H)-carboxylate (9-e). The enantiomers of 9-e are separated using supercritical fluid chromatography to yield tert-butyl (S)-4-(3-carbamoyl-2-(4-phenoxyphenyl)-4,5,6,7-tetrahydropyrazolo[1,5-a]pyrimidin-7-yl)-3,6-dihydropyridine-1(2H)-carboxylate (9-f). 9-f undergoes hydroxylation with AD-Mixα and methanesulfonamide in tert-butyl alcohol and water to yield tert-butyl (3S,4S)-4-((S)-3-carbamoyl-2-(4-phenoxyphenyl)-4,5,6,7-tetrahydropyrazolo[1,5-a]pyrimidin-7-yl)-3,4-dihydroxypiperidine-1-carboxylate (9-g) (alternatively, reaction with AD-Mixβ results in the addition of hydroxy groups with opposite stereochemistry). A hydroxyl substituent of 9-g is reduced with triethylsilane and trifluoroacetic acid in dichloromethane to yield tert-butyl (3R)-4-((S)-3-carbamoyl-2-(4-phenoxyphenyl)-4,5,6,7-tetrahydropyrazolo[1,5-a]pyrimidin-7-yl)-3-hydroxypiperidine-1-carboxylate (9-h), in which the enantiomers are separated by supercritical fluid chromatography to yield tert-butyl (3R,4R)-4-((S)-3-carbamoyl-2-(4-phenoxyphenyl)-4,5,6,7-tetrahydropyrazolo[1,5-a]pyrimidin-7-yl)-3-hydroxypiperidine-1-carboxylate (9-i) and tert-butyl (3R,4S)-4-((S)-3-carbamoyl-2-(4-phenoxyphenyl)-4,5,6,7-tetrahydropyrazolo[1,5-a]pyrimidin-7-yl)-3-hydroxypiperidine-1-carboxylate (9-j). The amine of the two isomers are each deprotected and reacted with acryloyl chloride (1-g) in the presence of sodium bicarbonate in THF/H₂O to yield (S)-7-((3R,4R)-1-acryloyl-3-hydroxypiperidin-4-yl)-2-(4-phenoxyphenyl)-4,5,6,7-tetrahydropyrazolo[1,5-a]pyrimidine-3-carboxamide (9-A) and (S)-7-((3R,4S)-1-acryloyl-3-hydroxypiperidin-4-yl)-2-(4-phenoxyphenyl)-4,5,6,7-tetrahydropyrazolo[1,5-a]pyrimidine-3-carboxamide (9-A′).

### Example S34. ((7S)-7-(1-acryloyl-3,3-dimethylpiperidin-4-yl)-2-(4-phenoxyphenyl)-4,5,6,7-tetrahydropyrazolo[1,5-a]pyrimidine-3-carboxamide (9-B)

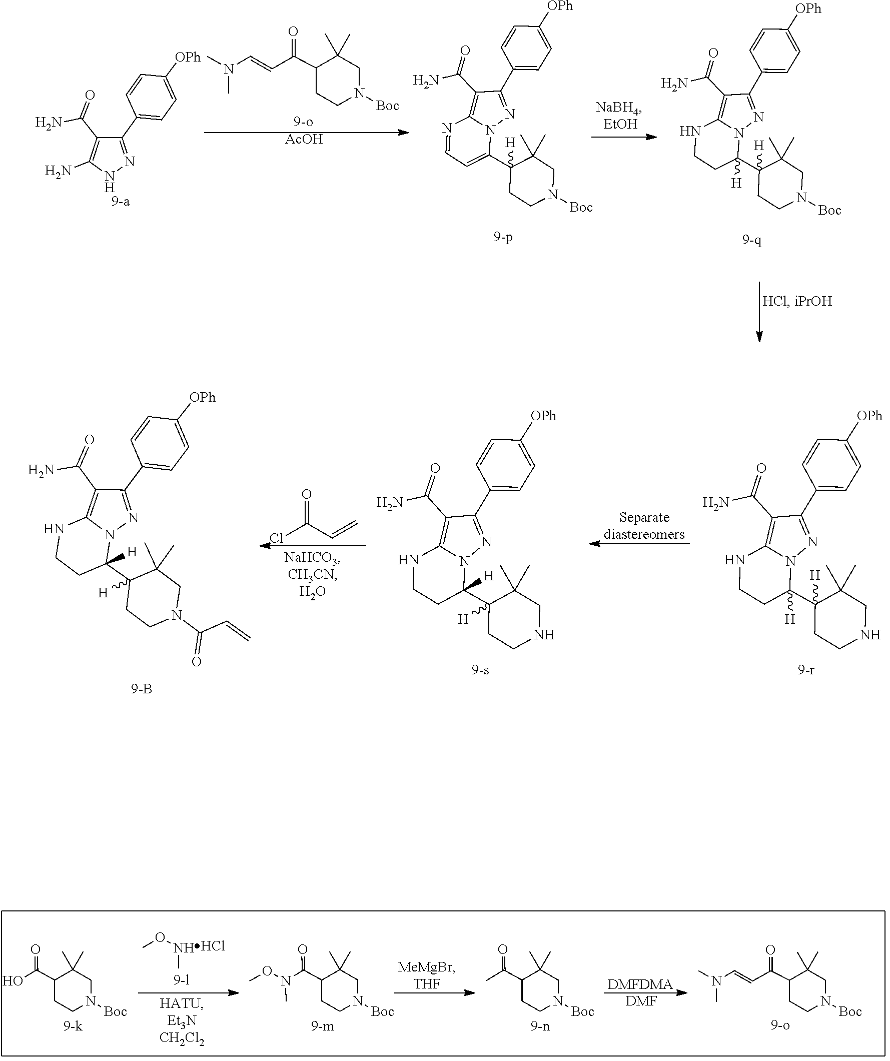

Reacting 1-(tert-butoxycarbonyl)-3,3-dimethylpiperidine-4-carboxylic acid (9-k) with N,O-dimethylhydroxylamine hydrochloride (9-1) in the presence of HATU and triethylamine in dichloromethane yields tert-butyl 4-(methoxy(methyl)carbamoyl)-3,3-dimethylpiperidine-1-carboxylate (9-m), which is subsequently converted to tert-butyl 4-acetyl-3,3-dimethylpiperidine-1-carboxylate (9-n) by reacting with MeMgBr in THF. 9-n reacts with N,N-dimethylformamide dimethyl acetal in DMF to yield tert-butyl (E)-4-(3-(dimethylamino)acryloyl)-3,3-dimethylpiperidine-1-carboxylate (9-o). The coupling between 5-amino-3-(4-phenoxyphenyl)-1H-pyrazole-4-carboxamide (9-a) and 9-o in acetic acid yields tert-butyl 4-(3-carbamoyl-2-(4-phenoxyphenyl)pyrazolo[1,5-a]pyrimidin-7-yl)-3,3-dimethylpiperidine-1-carboxylate (9-p), which is reduced with sodium borohydride in ethanol to yield tert-butyl 4-(3-carbamoyl-2-(4-phenoxyphenyl)-4,5,6,7-tetrahydropyrazolo[1,5-a]pyrimidin-7-yl)-3,3-dimethylpiperidine-1-carboxylate (9-q). The Boc-protected amine of 9-q is deprotected in acidic conditions to yield 7-(3,3-dimethylpiperidin-4-yl)-2-(4-phenoxyphenyl)-4,5,6,7-tetrahydropyrazolo[1,5-a]pyrimidine-3-carboxamide (9-r), in which the enantiomers are separated by supercritical fluid chromatography to (7S)-7-(3,3-dimethylpiperidin-4-yl)-2-(4-phenoxyphenyl)-4,5,6,7-tetrahydropyrazolo[1,5-a]pyrimidine-3-carboxamide (9-s). 9-s is subsequently reacted with acryloyl chloride (1-g) in the presence of a sodium bicarbonate base in acetonitrile/H₂O to yield (7S)-7-(1-acryloyl-3,3-dimethylpiperidin-4-yl)-2-(4-phenoxyphenyl)-4,5,6,7-tetrahydropyrazolo[1,5-a]pyrimidine-3-carboxamide (9-B).

### Example S35. (7S)-7-(1-acryloyl-3,3-difluoropiperidin-4-yl)-2-(4-phenoxyphenyl)-4,5,6,7-tetrahydropyrazolo[1,5-a]pyrimidine-3-carboxamide (9-C)

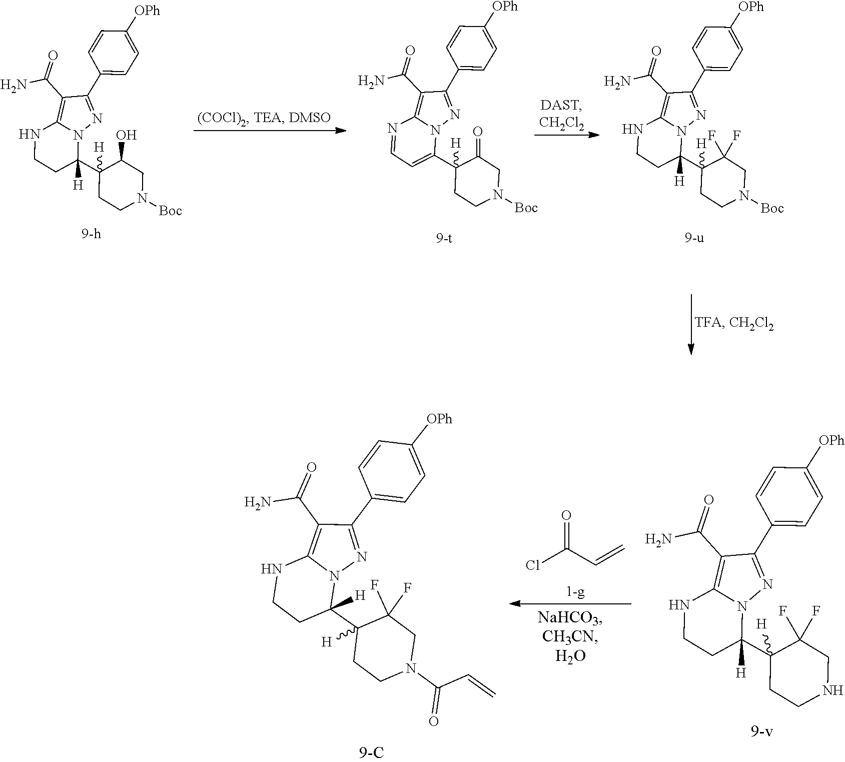

The alcohol of tert-butyl (3R)-4-((S)-3-carbamoyl-2-(4-phenoxyphenyl)-4,5,6,7-tetrahydropyrazolo[1,5-a]pyrimidin-7-yl)-3-hydroxypiperidine-1-carboxylate (9-h) is oxidized by reacting with oxalyl chloride followed by triethylamine in DMSO to yield tert-butyl 4-((S)-3-carbamoyl-2-(4-phenoxyphenyl)-4,5,6,7-tetrahydropyrazolo[1,5-a]pyrimidin-7-yl)-3-oxopiperidine-1-carboxylate (9-t). 9-t is reacted with diethylaminosulfur trifluoride in dichloromethane to yield tert-butyl 4-((S)-3-carbamoyl-2-(4-phenoxyphenyl)-4,5,6,7-tetrahydropyrazolo[1,5-a]pyrimidin-7-yl)-3,3-difluoropiperidine-1-carboxylate (9-u), and subsequently the the Boc-protected amine of 9-u is deprotected in acidic conditions to yield (7S)-7-(3,3-difluoropiperidin-4-yl)-2-(4-phenoxyphenyl)-4,5,6,7-tetrahydropyrazolo[1,5-a]pyrimidine-3-carboxamide (9-v). 9-v is reacted with acryloyl chloride (1-g) in the presence of a sodium bicarbonate base in acetonitrile/H₂O to yield (7S)-7-(1-acryloyl-3,3-difluoropiperidin-4-yl)-2-(4-phenoxyphenyl)-4,5,6,7-tetrahydropyrazolo[1,5-a]pyrimidine-3-carboxamide (9-C).

What is claimed is:

1. A compound of Formula (1): 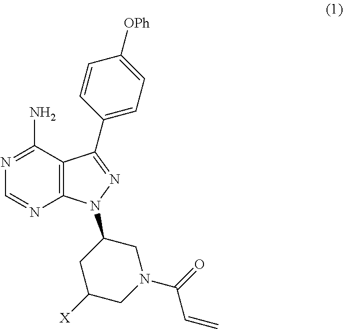
or a pharmaceutically acceptable salt thereof, wherein:
X is chosen from —OH, —OY, —Y₂, and -(halo)₂, and Y is C₁-C₄ alkyl optionally substituted with 1 to 3 halo.

2. A compound of Formula (2): 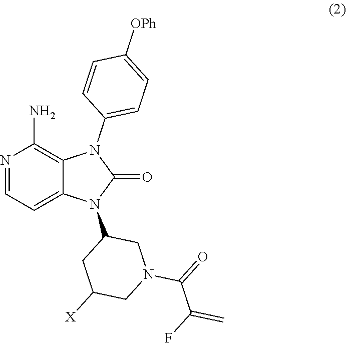
or a pharmaceutically acceptable salt thereof, wherein:
X is chosen from —OH, —OY, —Y₂, and -(halo)₂, and Y is C₁-C₄ alkyl optionally substituted with 1 to 3 halo.

3. A compound of Formula (3): 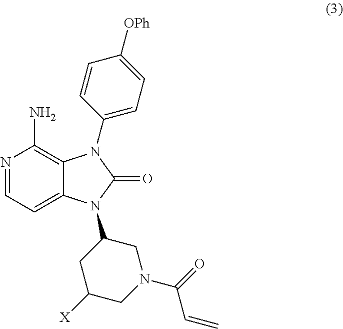
or a pharmaceutically acceptable salt thereof, wherein:
X is chosen from —OH, —OY, —Y₂, and -(halo)₂, and Y is C₁-C₄ alkyl optionally substituted with 1 to 3 halo.

4. A compound of Formula (4): 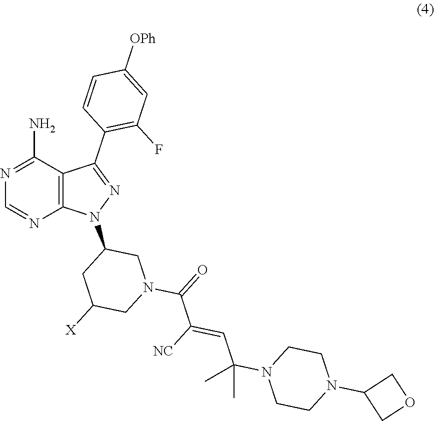
or a pharmaceutically acceptable salt thereof, wherein:
X is chosen from —OH, —OY, —Y₂, and -(halo)₂, and Y is C₁-C₄ alkyl optionally substituted with 1 to 3 halo.

5. A compound of Formula (5): 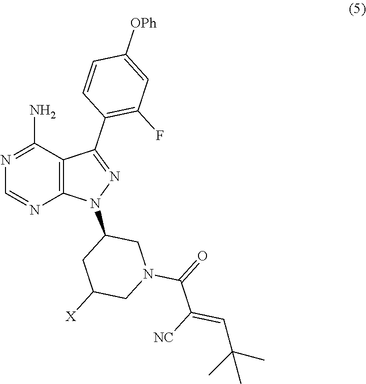
or a pharmaceutically acceptable salt thereof, wherein:
X is chosen from —OH, —OY, —Y₂, and -(halo)₂, and Y is C₁-C₄ alkyl optionally substituted with 1 to 3 halo.

6. A compound of Formula (6): 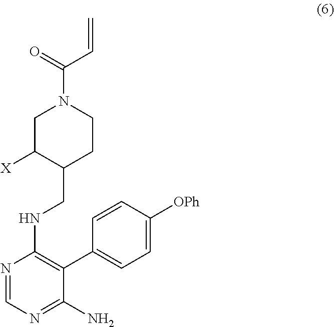
or a pharmaceutically acceptable salt thereof, wherein:
X is chosen from —OH, —OY, —Y₂, and -(halo)₂, and Y is C₁-C₄ alkyl optionally substituted with 1 to 3 halo.

7. A compound of Formula (7): 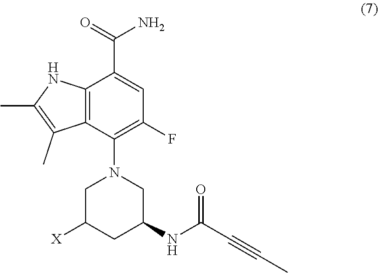
or a pharmaceutically acceptable salt thereof, wherein:
X is chosen from —OH, —OY, —Y₂, and -(halo)₂, and Y is C₁-C₄ alkyl optionally substituted with 1 to 3 halo.

8. A compound of Formula (8): 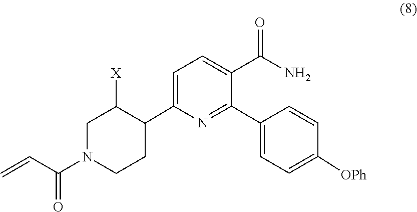
or a pharmaceutically acceptable salt thereof, wherein:
X is chosen from —OH, —OY, —Y₂, and -(halo)₂, and Y is C₁-C₄ alkyl optionally substituted with 1 to 3 halo.

9. A compound of Formula (9): 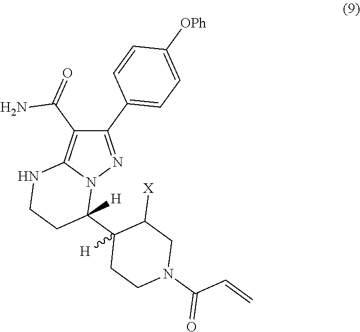
or a pharmaceutically acceptable salt thereof, wherein:
X is chosen from —OH, —OY, —Y₂, and -(halo)₂, and Y is C₁-C₄ alkyl optionally substituted with 1 to 3 halo.

10. The compound of any one of claims 1 to 9, wherein Y is chosen from methyl, ethyl, CH(halo)₂, and C(halo)₃.

11. The compound of any one of claims 1 to 10, wherein halo is F.

12. The compound of any one of claims 1 to 11 chosen from:

| Com- | |
|---|---|
| pound | |
| No. | Structure |
| 1-A | 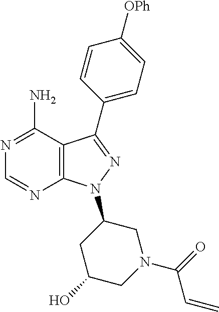 [SMILES: C=CC(=O)N1C[C@H](O)C[C@@H](n2nc(-c3ccc(Oc4ccccc4)cc3)c3c(N)ncnc32)C1] |
| 1-B | 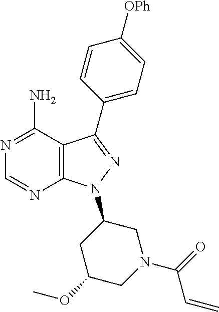 [SMILES: C=CC(=O)N1C[C@H](OC)C[C@@H](n2nc(-c3ccc(Oc4ccccc4)cc3)c3c(N)ncnc32)C1] |
| 1-C | 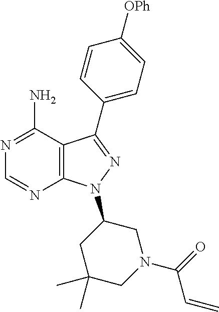 [SMILES: C=CC(=O)N1C[C@H](n2nc(-c3ccc(Oc4ccccc4)cc3)c3c(N)ncnc32)CC(C)(C)C1] |
| 1-D | 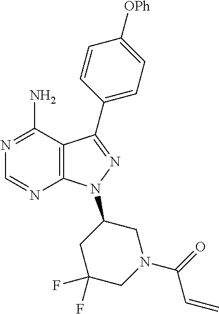 [SMILES: C=CC(=O)N1C[C@H](n2nc(-c3ccc(Oc4ccccc4)cc3)c3c(N)ncnc32)CC(F)(F)C1] |
| 2-A | 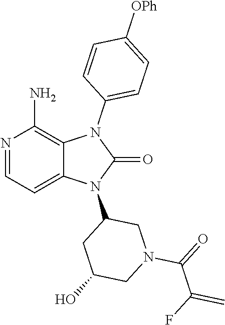 |
| 2-B | 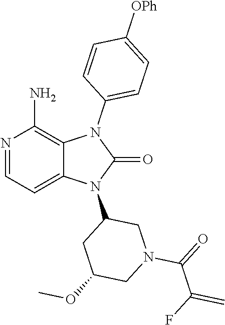 |
| 2-C | 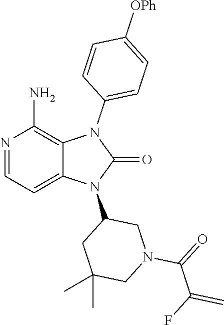 |
| 2-D | 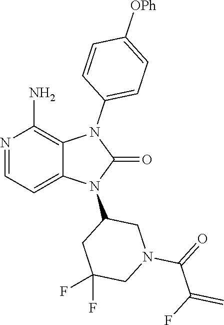 |
| 3-A | 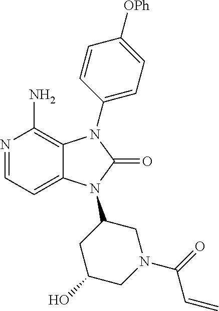 |
| 3-B | 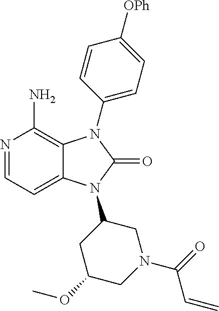 |
| 3-C | 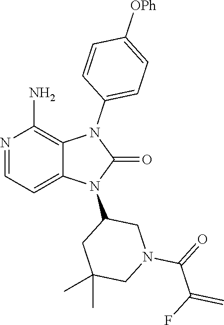 |
| 3-D | 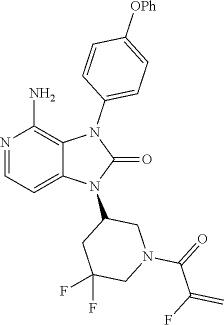 |
| 4-A | 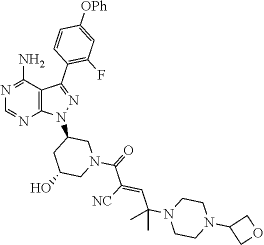 [SMILES: CC(C)(/C=C(\C#N)C(=O)N1C[C@H](O)C[C@@H](n2nc(-c3ccc(Oc4ccccc4)cc3F)c3c(N)ncnc32)C1)N1CCN(C2COC2)CC1] |
| 4-B | 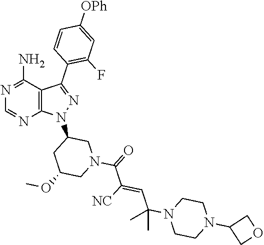 [SMILES: CO[C@@H]1C[C@@H](n2nc(-c3ccc(Oc4ccccc4)cc3F)c3c(N)ncnc32)CN(C(=O)/C(C#N)=C/C(C)(C)N2CCN(C3COC3)CC2)C1] |
| 4-C | 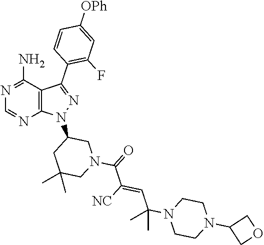 [SMILES: CC1(C)C[C@@H](n2nc(-c3ccc(Oc4ccccc4)cc3F)c3c(N)ncnc32)CN(C(=O)/C(C#N)=C/C(C)(C)N2CCN(C3COC3)CC2)C1] |
| 4-D | 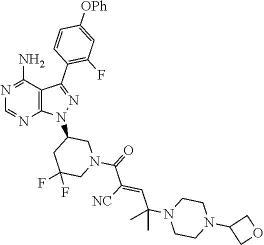 [SMILES: CC(C)(/C=C(\C#N)C(=O)N1C[C@H](n2nc(-c3ccc(Oc4ccccc4)cc3F)c3c(N)ncnc32)CC(F)(F)C1)N1CCN(C2COC2)CC1] |
| 5-A | 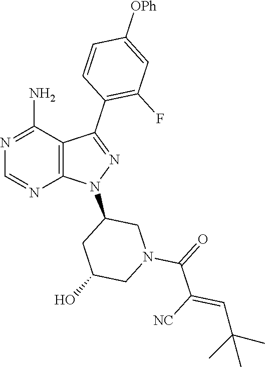 [SMILES: CC(C)(C)/C=C(\C#N)C(=O)N1C[C@H](O)C[C@@H](n2nc(-c3ccc(Oc4ccccc4)cc3F)c3c(N)ncnc32)C1] |
| 5-B | 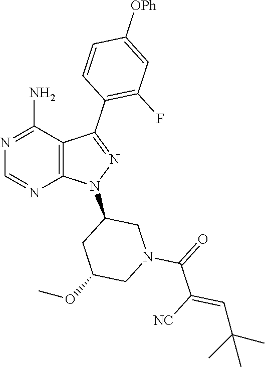 [SMILES: CO[C@@H]1C[C@@H](n2nc(-c3ccc(Oc4ccccc4)cc3F)c3c(N)ncnc32)CN(C(=O)/C(C#N)=C/C(C)(C)C)C1] |
| 5-C | 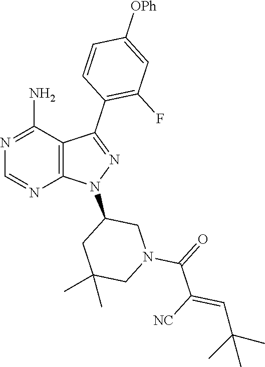 [SMILES: CC(C)(C)/C=C(\C#N)C(=O)N1C[C@H](n2nc(-c3ccc(Oc4ccccc4)cc3F)c3c(N)ncnc32)CC(C)(C)C1] |
| 5-D | 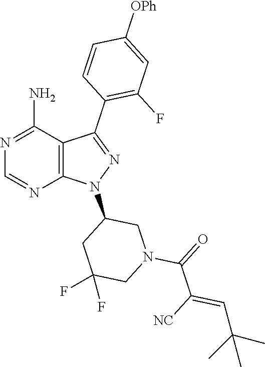 [SMILES: CC(C)(C)/C=C(\C#N)C(=O)N1C[C@H](n2nc(-c3ccc(Oc4ccccc4)cc3F)c3c(N)ncnc32)CC(F)(F)C1] |
| 6-A | 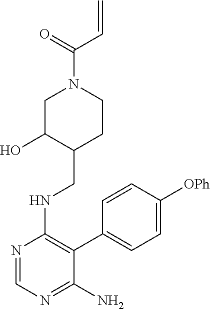 [SMILES: C=CC(=O)N1CCC(CNc2ncnc(N)c2-c2ccc(Oc3ccccc3)cc2)C(O)C1] |
| 6-B | 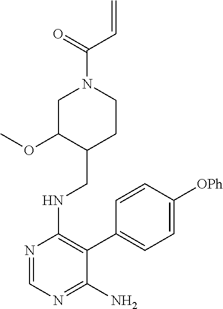 [SMILES: C=CC(=O)N1CCC(CNc2ncnc(N)c2-c2ccc(Oc3ccccc3)cc2)C(OC)C1] |
| 6-C | 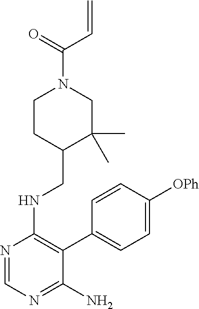 [SMILES: C=CC(=O)N1CCC(CNc2ncnc(N)c2-c2ccc(Oc3ccccc3)cc2)C(C)(C)C1] |
| 6-D | 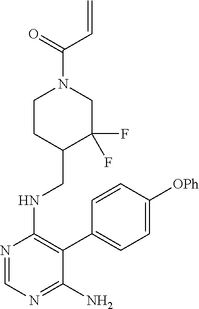 [SMILES: C=CC(=O)N1CCC(CNc2ncnc(N)c2-c2ccc(Oc3ccccc3)cc2)C(F)(F)C1] |
| 7-A | 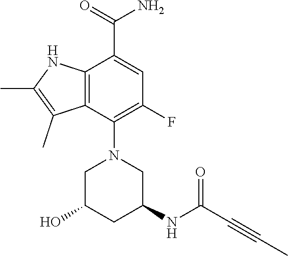 [SMILES: CC#CC(=O)N[C@H]1C[C@H](O)CN(c2c(F)cc(C(N)=O)c3[nH]c(C)c(C)c23)C1] |
| 7-B | 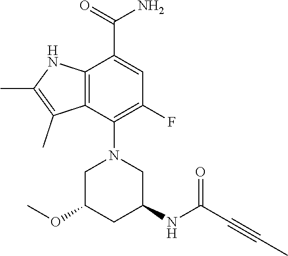 [SMILES: CC#CC(=O)N[C@H]1C[C@H](OC)CN(c2c(F)cc(C(N)=O)c3[nH]c(C)c(C)c23)C1] |
| 7-C | 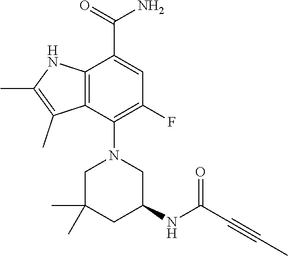 [SMILES: CC#CC(=O)N[C@@H]1CN(c2c(F)cc(C(N)=O)c3[nH]c(C)c(C)c23)CC(C)(C)C1] |
| 7-D | 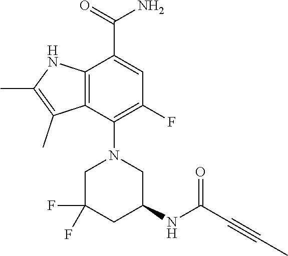 [SMILES: CC#CC(=O)N[C@@H]1CN(c2c(F)cc(C(N)=O)c3[nH]c(C)c(C)c23)CC(F)(F)C1] |
| 8-A | 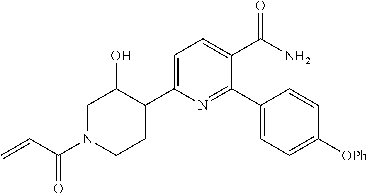 [SMILES: C=CC(=O)N1CCC(c2ccc(C(N)=O)c(-c3ccc(Oc4ccccc4)cc3)n2)C(O)C1] |
| 8-B | 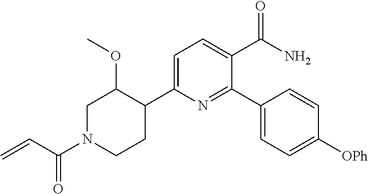 [SMILES: C=CC(=O)N1CCC(c2ccc(C(N)=O)c(-c3ccc(Oc4ccccc4)cc3)n2)C(OC)C1] |
| 8-C | 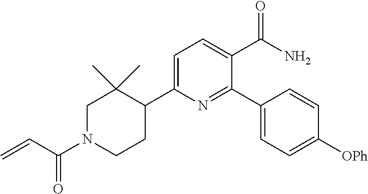 [SMILES: C=CC(=O)N1CCC(c2ccc(C(N)=O)c(-c3ccc(Oc4ccccc4)cc3)n2)C(C)(C)C1] |
| 8-D | 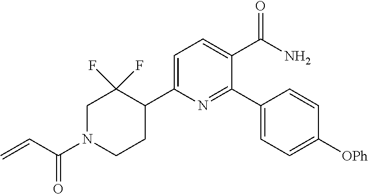 [SMILES: C=CC(=O)N1CCC(c2ccc(C(N)=O)c(-c3ccc(Oc4ccccc4)cc3)n2)C(F)(F)C1] |
| 9-A & 9-A′ | 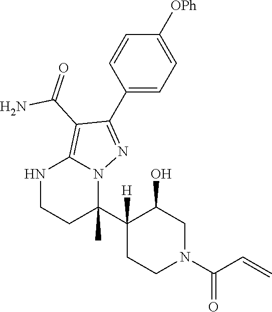 [SMILES: C=CC(=O)N1CC[C@H]([C@]2(C)CCNc3c(C(N)=O)c(-c4ccc(Oc5ccccc5)cc4)nn32)[C@@H](O)C1] |
| | 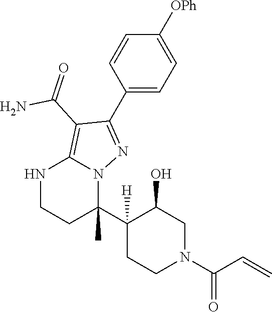 [SMILES: C=CC(=O)N1CC[C@@H]([C@]2(C)CCNc3c(C(N)=O)c(-c4ccc(Oc5ccccc5)cc4)nn32)[C@@H](O)C1] |
| 9-B | 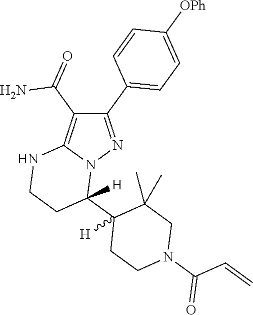 [SMILES: C=CC(=O)N1CCC([C@@H]2CCNc3c(C(N)=O)c(-c4ccc(Oc5ccccc5)cc4)nn32)C(C)(C)C1] |
| 9-C | 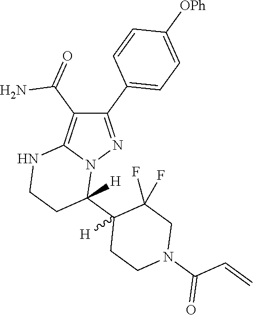 [SMILES: C=CC(=O)N1CCC([C@@H]2CCNc3c(C(N)=O)c(-c4ccc(Oc5ccccc5)cc4)nn32)C(F)(F)C1] |

or a pharmaceutically acceptable salt thereof.

13. A composition comprising a pharmaceutically acceptable excipient and at least one compound chosen from any one of claims 1 to 12.

14. A method of treating a disease or disorder mediated by BTK comprising administering to a patient in need thereof an effective amount of a compound chosen from any one of claims 1 to 12, or a composition according to claim 13.

15. A method of preparing a compound of Formula (3-A): 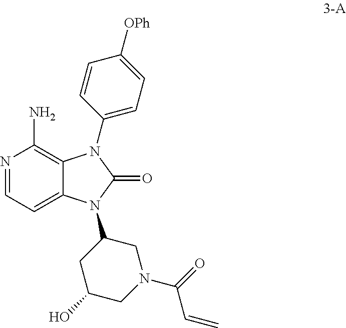
comprising:
(a) coupling a compound of Formula (2-a): 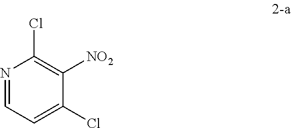 [SMILES: O=[N+]([O-])c1c(Cl)ccnc1Cl]
with a compound of Formula (2-b): 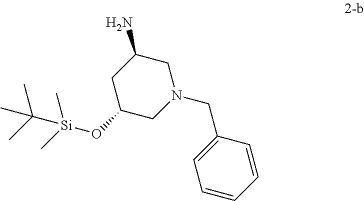 [SMILES: CC(C)(C)[Si](C)(C)O[C@@H]1C[C@@H](N)CN(Cc2ccccc2)C1]
to provide a compound of Formula (2-c): 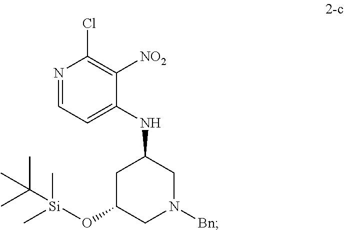 [SMILES: CC(C)(C)[Si](C)(C)O[C@@H]1C[C@@H](Nc2ccnc(Cl)c2[N+](=O)[O-])CN(Cc2ccccc2)C1]
(b) coupling the compound of Formula (2-c) with the compound of Formula (2-d): 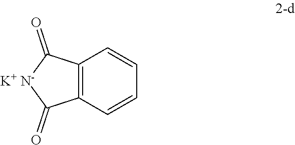
to provide a compound of Formula (2-e): 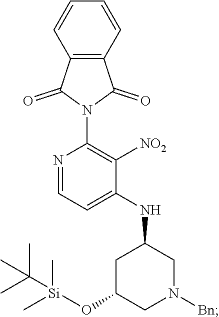 [SMILES: CC(C)(C)[Si](C)(C)O[C@@H]1C[C@@H](Nc2ccnc(N3C(=O)c4ccccc4C3=O)c2[N+](=O)[O-])CN(Cc2ccccc2)C1]
(c) undergoing a ring-formation reaction in the compound of Formula (2-e) to provide a compound of Formula (2-f): 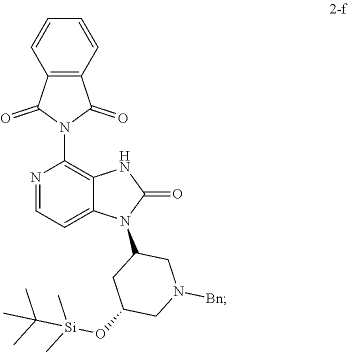
(d) coupling the compound of Formula (2-f) with the compound of Formula (1-b): 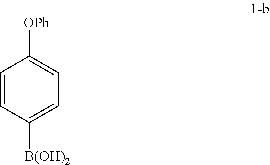 [SMILES: OB(O)c1ccc(Oc2ccccc2)cc1]
to provide a compound of Formula (2-h): 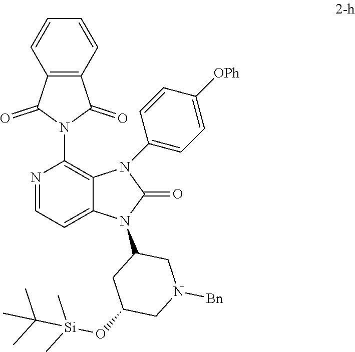
(e) deprotecting the nitrogen substituted on the pyridine of the compound of Formula (2-h) to provide a compound of Formula (2-i): 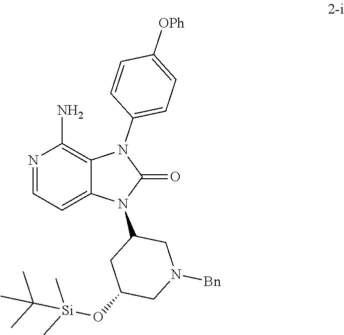
(f) deprotecting the piperidine nitrogen of the compound of Formula (2-i) to provide a compound of Formula (2-j): 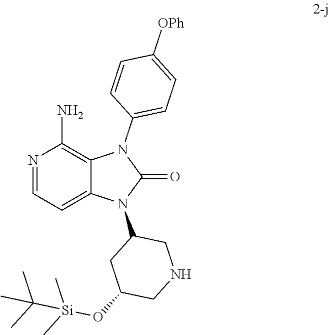
(g) coupling the compound of Formula (2-j) with the compound of Formula (1-g): 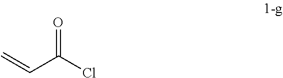
to provide a compound of Formula (3-b): 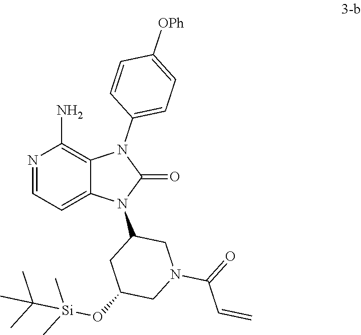
(h) deprotecting the oxygen of the compound of Formula (3-b) to provide a compound of Formula (3-A).
